(19) United States
(12) Patent Application Publication
(10) Pub. No.: US 2008/0286771 A1
(43) Pub. Date: Nov. 20, 2008

(54) Method of Predicting the Responsiveness of a Tumour to Erbb Receptor Drugs

(76) Inventors: Kevin Hudson, Cheshire (GB); Marie Caroline South, Cheshire (GB); Gayle Marshall, Cheshire (GB); Mehran Sam, Boxborough, MA (US)

Correspondence Address: MORGAN LEWIS & BOCKIUS LLP, 1111 PENNSYLVANIA AVENUE NW, WASHINGTON, DC, 20004, US

(21) Appl. No.: 11/658,159

(22) PCT Filed: Jul. 20, 2005
(86) PCT No.: PCT/GB05/02852

§ 371 Date: Jan. 23, 2007

Related U.S. Application Data
(60) Provisional application No. 60/590,357, filed on Jul. 23, 2004.
(60) Provisional application No. 60/619,027, filed on Oct. 18, 2004.

(51) Int. Cl. C12Q 1/68

(52) U.S. Cl. 435/6

(57) ABSTRACT

The invention relates to a method of selecting a mammal having or suspected of having a tumour for treatment with an erbB receptor drug which comprises testing a biological sample from the mammal for expression of anyone of the genes listed in Table 1 or 2 as defined herein whereby to predict an increased likelihood of response to the erbB receptor drug. Preferred genes include anyone of NES, GSPT2, ETR101, TAZ, CHST7, DNAJC3, NPAS2, PIN1, TCEA2, VAMP4, DAPK1, DAPK2, MLLT3, TNNC1, KIAA0931, ACOX2, EMP1, SLC20A1, SPRY2 or PGM1.

The present invention relates to sensitivity of tumours to therapeutic agents which can be predicted from the gene expression profile of the tumour and hence that the suitability of cancer patients for treatment with such therapeutic agents can be determined by measuring the relative expression levels of particular genes in tumour tissue.

The phosphorylation of proteins on tyrosine residues is a key element of signal transduction within cells. Enzymes capable of catalysing such reactions are termed tyrosine kinases. A number of these enzymes exist as integral components of transmembrane receptor molecules and are classified as receptor tyrosine kinases (RTKs). There are several members of this family of RTKs, class I of which includes the erbB family, e.g. epidermal growth factor receptor (BGFR), erbB2, erbB3 and erbB4. Binding of a variety of ligands to the external domain activates the EGFR tyrosine kinase domain. Activation causes EGFR itself and a number of cellular substrates to become phosphorylated on tyrosine residues. These phosphorylation reactions are a major component of growth factor induced proliferation of cells.

The erbB family of receptor tyrosine kinases are known to be frequently involved in driving the proliferation and survival of tumour cells (reviewed in Olayioye et al., EMBO J., 2000, 19, 3159). One mechanism by which this can occur is over expression of the receptor at the protein level, for example as a result of gene amplification. This has been observed in many common human cancers (reviewed in Klapper et al., Adv. Cancer Res., 2000, 77, 25) such as, non-small cell lung cancers (NSCLCs) including adenocarcinomas (Cerny et al., Brit. J. Cancer, 1986, 54, 265; Reubi et al., Int. J. Cancer, 1990, 45, 269; Rusch et al., Cancer Research, 1993, 53, 2379; Brabender et al, Clin. Cancer Res., 2001, 7, 1850) as well as other cancers of the lung (Hendler et al., Cancer Cells, 1989, 7, 347.

It is now several decades since the study of retroviral mediated cellular transformation began to revolutionize our understanding of malignant transformation. Transformation was shown to be dependent on oncogenes carried by viruses and these were shown to have mammalian cellular counterparts, proto-oncogenes. In 1984, EGFR was described as the mammalian counterpart of the retroviral oncogene, v-erbB (Downward et al). This, coupled to earlier observations describing a two component autocrine growth promoting mechanism in cancer cells consisting of EGF ligand and its receptor EGFR (Sporn & Todaro), strengthened the hypothesis that EGFR signalling is an important contributor to tumourigenesis. Subsequent reports continued to provide evidence that EGFR is an attractive target for therapeutic intervention in Cancer (see Yarden & Sliwkowski for review). EGFR is markedly overexpressed across a large variety of epithelial Cancers (see Salomon et al) and some immunohistochemical studies have demonstrated EGFR expression is associated with poor prognosis. In addition to overexpression, it is recognised that there is potential for deregulated EGFR signalling in tumours via a number of alternative mechanisms including i) EGFR mutations ii) increased ligand expression and enhanced autocrine loop and iii) heterodimerisation and cross talk with other erbB receptor family members.

In addition, a wealth of pre-clinical information suggests that the erbB family of receptor tyrosine kinases are involved in cellular transformation. In addition to this, a number of pre-clinical studies have demonstrated that anti-proliferative effects can be induced by knocking out one or more erbB activities by small molecule inhibitors, dominant negatives or inhibitory antibodies (reviewed in Mendelsohn et al., Oncogene, 2000, 19, 6550).

Thus it has been recognised that inhibitors of these receptor tyrosine kinases should be of value as a selective inhibitor of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19, 6550-6565).

A number of small molecule inhibitors of erbB family of receptor tyrosine kinases are known, particularly inhibitors of EGF and erbB2 receptor tyrosine kinases. For example European Patent Application No. 0566226 and International Patent Applications WO 96/33980 and WO 97/30034 disclose that certain quinazoline derivatives which possess an amino substituent at the 4-position possess EGFR tyrosine kinase inhibitory activity and are inhibitors of cancer tissue.

It has been disclosed by J R Woodburn et al. in Proc. Amer. Assoc. Cancer Research, 1997, 38, 633 and Pharmacol. Ther., 1999, 82, 241-250 that the compound N-(3-chloro-4-fluorophenyl)-7-methoxy-6-(3-morpholinopropoxy)quinazolin-4-amine is a potent BGFR tyrosine kinase inhibitor. This compound is also known as Iressa (registered trade mark), gefitinib (United States Adopted Name), by way of the code number ZD1839 and Chemical Abstracts Registry Number 184475-35-2. The compound is principally identified hereinafter as gefitinib.

Gefitinib was developed as an inhibitor of epidermal growth factor receptor-tyrosine kinase (EGFR-TK), which blocks signaling pathways responsible for driving proliferation, invasion, and survival of cancer cells (Wakeling, A. E., et al. Cancer Res, 2002, 62(20), p 5749). Gefitinib has provided clinical validation of small molecule inhibitors of EGFR. Potent anti-tumour effects as well as rapid improvements in NSCLC-related symptoms and quality of life have been observed in clinical studies that enrolled patients with advanced NSCLC who did not respond to platinum-based chemotherapy. The Phase II ‘IDEAL’ trials demonstrated that single agent gefitinib resulted in objective anti-tumour activity, symptomatic improvement and limited toxicity in patients with advanced NSCLC and previously treated with cytotoxic chemotherapy (Fukuoka et al., Kris et al). Objective response rate (Complete Response+Partial Response) was 18.4% and 11.8% respectively in the IDEAL 1 and IDEAL 2 trials. The differences in response in these clinical trials has been attributed to different population groups in the two trials, predominantly Japanese in IDEAL 1 and a predominantly European-derived population in IDEAL 2. Beyond objective responses, additional patients experienced stable disease and/or symptom improvement meaning that approximately 50% of patients overall benefit from gefitinib. The tumour response data has been the basis of initial regulatory approvals of gefitinib in advanced NSCLC in several markets.

It is important to be able to understand the basis of response to anti-cancer therapeutic agents such as gefitinib since this would allow clinicians to maximise the benefit/risk ratio for each patient, potentially via the development of diagnostic tests to identify patients most likely to benefit from gefitinib treatment. An obvious candidate marker of response to gefitinib has been EGFR expression level. However, gefitinib inhibition of growth of some cancer-derived cell lines and tumour xenografts is not well correlated with the level of expression of EGFR. Furthermore, studies alongside the IDEAL trials demonstrated that EGFR protein expression as measured by IHC was not an accurate predictor of response to gefitinib (Bailey et al). Although there are now several additional hypotheses based on genetics, genomics, proteomics, biochemical and other studies, there is still no pre-treatment predictive biomarker of gefitinib response currently approved by regulatory authorities. Possibly the most significant recent breakthrough in understanding gefitinib response has come from recent data (Lynch et al, Paez et al) indicating that mutation in the BGFR kinase domain predicts gefitinib hypersensitivity in NSCLC patients. Hypersensitivity is a vague term but in this field is generally understood to mean patients experiencing objective tumour responses (i.e. marked tumour regression, normally above 50%). As well as demonstrating the EGFR mechanism of action for gefitinib, this may provide a basis for venturing into other disease settings such as first line, adjuvant and possibly earlier cancer intervention with EGFR inhibitors in a targeted subpopulation in NSCLC patients and other types of cancers carrying the EGFR mutation.

However, it is likely that restricting prescription of gefitinib to the mutant EGFR carrying tumour subgroup will deprive many patients who could benefit from gefitinib. Firstly there are emerging reports of gefitinib hypersensitive patients with undetectable EGFR mutation in their tumour and other patients with EGFR mutation who do not respond to gefitinib. Secondly, data reported at ASCO 2004 (Shepherd et al) indicated that the EGFR small molecule tyrosine kinase inhibitor erlotinib (Roche, Genentech, OSI) prolongs survival in advanced NSCLC previously treated with chemotherapy, by ˜2 months across the population with resulting 41% reduction in risk of death at one year. Most interestingly, the survival benefit appears to be is derived from patients in the stable disease response population as well as hypersensitive patients. This highlights the likely importance of identifying likely gefitinib responsive patients beyond those carrying EGFR mutation. Definitive survival benefit is also likely to be demonstrated from ongoing clinical trials with gefitinib.

The differential response of patients to chemotherapy treatments indicates that there is a need to find methods of predicting which treatment regimes best suit a particular patient.

There is an increasing body of evidence that suggests that patients' responses to numerous drugs may be related to a patients' genetic, genomic, proteomic, biochemical or profile and that determination of the genetic factors that influence, for example, response to a particular drug could be used to provide a patient with a personalised treatment regime. Such personalised treatment regimes offer the potential to maximise therapeutic benefit to the patient, whilst minimising, for example side effects that may be associated with alternative and less effective treatment regimes.

Therefore there is a need for methods that can predict a patients' response to a drug based on the results of a test that indicates whether the patient is likely to respond to treatment or to be resistant to treatment.

The present invention is based on the discovery that the sensitivity of tumours to therapeutic agents can be predicted from the gene expression profile of the tumour and hence that the suitability of tumour patients for treatment with such therapeutic agents can be determined by measuring the relative expression levels of particular genes in tumour tissue.

According to one aspect of the present invention there is provided a method of selecting a mammal having or suspected of having a tumour for treatment with an erbB receptor drug which comprises testing a biological sample from the mammal for expression of any one of the genes listed in Table 1 as defined herein whereby to predict an increased likelihood of response to the erbB receptor drug.

According to another aspect of the present invention there is provided a method of selecting a mammal having or suspected of having a tumour for treatment with an erbB receptor drug which comprises testing a biological sample from the mammal for expression of any one of the genes listed in Table 1 or DAPK2 whereby to predict an increased likelihood of response to the erbB receptor drug.

In one embodiment the method comprises testing a biological sample from the mammal for expression of any one of ACOX2, NPAS2, NES, CHST7, GSPT2, DAPK1, DAPK2 or TNNC1. More preferably the method comprises testing a biological sample from the mammal for expression of any one of NPAS2, NES, CHST7 or DAPK1. More preferably the method comprises testing a biological sample from the mammal for expression of at least two of NPAS2, NES, CHST7 or DAPK1. More preferably the method comprises testing a biological sample from the mammal for expression of at least three of NPAS2, NES, CHST7 or DAPK1. More preferably still the method comprises testing a biological sample from the mammal for expression of NPAS2, NES, CHST7 and DAPK1.

In an alternative embodiment the method comprises testing a biological sample from the mammal for expression of any one of NES, GSPT2, ETR101, TAZ, CHST7, DNAJC3, NPAS2, PIN1, TCEA2, VAMP4, DAPK1, DAPK2, MLLT3, TNNC1 or KIAA0931. More preferably the method comprises testing a biological sample from the mammal for expression of any one of DAPK1, DAPK2 or NES. More preferably the method comprises testing a biological sample from the mammal for expression of at least two of DAPK1, DAPK2 or NES. More preferably the method comprises testing a biological sample from the mammal for expression of DAPK1, DAPK2 and NES.

In a preferred embodiment the method additionally comprises testing a biological sample from the mammal for expression of any gene listed in Table 2 as defined herein. More preferably the method comprises testing a biological sample from the mammal for expression of EMP1, SLC20A1, SPRY2 or PGM1. More preferably the method comprises testing a biological sample from the mammal for expression of EMP1.

In an alternative preferred embodiment the method additionally comprises testing a biological sample from the mammal for expression of any gene listed in Table 2 as defined herein. More preferably the method comprises testing a biological sample from the mammal for expression of EMP1, HCA127, UBL5, ZNF23, UROD, CD44, SPRY1, RAPGEF2, SLC20A1, NRP1, PGM1, SPRY2, PTGER3, SCN10A, KITLG, CDH1, HOP, BCL3 or OLFM1. More preferably the method comprises testing a biological sample from the mammal for expression of EMP1.

Preferably the tumour is selected from the group consisting of leukaemia, multiple myeloma, lymphoma, bile duct, bone, bladder, brain, CNS, glioblastoma, breast, colorectal, cervical, endometrial, gastric, head, neck, hepatic, lung, muscle, neuronal, oesophageal, ovarian, pancreatic, pleural membrane, peritoneal membrane, prostate, renal, skin, testicular, thyroid, uterine and vulval. More preferably the tumour is selected from one of non-small cell lung, pancreatic, head or neck. More preferably the tumour is selected from one of non-small cell lung, head or neck.

Preferably the erbB receptor drug is selected from any one of gefitinib, erlotinib, PKI-166, EKB-569, HKI-272, lapatinib, canertinib, AEE788, XL647, BMS 5599626, cetuximab, matuzumab, panitumumab, MR1-1, IMC-11F8 or EGFRL11. Most preferably the erbB receptor drug is gefitinib.

In a further preferred embodiment of the method of the invention the mammal is a human and the method comprises testing a biological sample from the human for increased expression of DAPK1 and decreased expression of NPAS2, NES, CHST7 or EMP1 whereby to predict an increased likelihood of response to gefitinib. In an alternative preferred embodiment of the method of the invention the mammal is a human and the method comprises testing a biological sample from the human for increased expression of DAPK1 and DAPK2 and decreased expression of NES and EMP1 whereby to predict an increased likelihood of response to gefitinib.

According to another aspect of the invention there is provided an isolated set of marker genes identified as having differential expression between tumour cells that are sensitive and resistant to an erbB receptor drug said gene set comprising one or more genes selected from at least the group consisting of the genes listed in Table 1 defined herein or DAPK2, including gene specific oligonucleotides derived from said genes. Preferably the set comprises at least 2 genes, more preferably at least 3 genes, more preferably at least 4 genes. More preferably the set comprises at least one gene selected from Table 2 as defined herein.

According to another aspect of the invention there is provided an isolated set of marker genes identified as having differential expression between tumour cells that are sensitive and resistant to an erbB receptor drug said gene set comprising one or more genes selected from at least the group consisting of the genes listed in Table 1 defined herein, including gene specific oligonucleotides derived from said genes. Preferably the set comprises at least 2 genes, more preferably at least 3 genes. More preferably the set comprises at least one gene selected from Table 2 as defined herein.

The present invention permits the improved selection of a patient, having or suspected of having a tumour, for treatment with an erbB receptor drug, in order to predict an increased likelihood of response to the erbB receptor drug.

In one embodiment, the method comprises testing a biological sample from the mammal for expression of at least one or more of the following from Table 1, which are found at lower levels in sensitive cells NPAS2, NES, CHST7, ACOX2 or GSPT2 or at least one or more of the following which are found at higher levels in sensitive cells DAPK1 or TNNC1. The Affymetrix ID and Affymetrix probe sequence for these genes are displayed in Table 1. In a preferred embodiment, the method further comprises testing a biological sample from the mammal for expression of DAPK2 which is found at higher levels in sensitive cells, whereby to predict an increased likelihood of response to the erbB receptor drug.

In an alternative embodiment, the method comprises testing a biological sample from the mammal for expression of at least one or more of the following from Table 1, which are found at lower levels in sensitive cells NES, GSPT2, ETR101, TAZ, CHST7, DNAJC3, NPAS2, PIN1, TCEA2 or VAMP4 or at least one or more of the following which are found at higher levels in sensitive cells DAPK1, DAPK2, MLLT3, TNNC1 or KIAA0931. The Affymetrix ID and Affymetrix probe sequence for these genes are displayed in Table 1.

In a preferred embodiment, the method further comprises testing a biological sample from the mammal for expression of any one of the genes listed in Table 2, whereby to predict an increased likelihood of response to the erbB receptor drug. In a preferred embodiment, the method comprises testing a biological sample from the mammal for expression of any one of the following genes listed in Table 2, which are found at lower levels in sensitive cells EMP1, SLC20A1, SPRY2 or PGM1, whereby to predict an increased likelihood of response to the erbB receptor drug. More preferably the method comprises testing a biological sample from the mammal for expression of EMP1.

In an alternative preferred embodiment, the method further comprises testing a biological sample from the mammal for expression of any one of the genes listed in Table 2, whereby to predict an increased likelihood of response to the erbB receptor drug. In a preferred embodiment, the method comprises testing a biological sample from the mammal for expression of any one of the following genes listed in Table 2, which are found at lower levels in sensitive cells EMP1, HCA127, UBL5, ZNF23, UROD, CD44, SPRY1, RAPGEF2, SLC20A1, NRP1, PGM1 or SPRY2 or at least one or more of the following which are found at higher levels in sensitive cells PTGER3, SCN10A, KITLG, CDH1, HOP, BCL3 or OLFM1 whereby to predict an increased likelihood of response to the erbB receptor drug. More preferably the method comprises testing a biological sample from the mammal for expression of EMP1.

In an especially preferred embodiment the method comprises testing a biological sample from the mammal for expression of NPAS2, NES, CHST7, DAPK1 and EMP1. High NPAS2, NES, CHST7 and EMP1 levels are associated with resistance to gefitinib and high DAPK1 levels are associated with sensitivity to gefitinib. Preferably, the assessment of expression comprises determination of whether DAPK1 levels are increased and NPAS2, NES, CHST7 and EMP1 levels are reduced.

In an alternative especially preferred embodiment the method comprises testing a biological sample from the mammal for expression of DAPK1, DAPK2, NES and EMP1. High EMP1 and NES levels are associated with resistance to gefitinib and high DAPK1 and DAPK2 levels are associated with sensitivity to gefitinib. Preferably, the assessment of expression comprises determination of whether DAPK1 and DAPK2 levels are increased and EMP1 and NES levels are reduced. In a most preferred embodiment the invention comprises determining the level of DAPK1 and EMP1.

According to another aspect of the invention there is provided a method for predicting clinical outcome of treatment with an erbB receptor drug for a mammal, having or suspected of having a tumour, comprising determining the level of any of the genes as described hereinabove in a biological sample taken from the tumour, or suspected tumour, wherein a poor outcome is predicted if:

a) the expression level of DAPK1 is reduced; and/or
b) the expression level of NPAS2, NES, CHST7 and EMP1 is increased.

According to another aspect of the invention there is provided a method for classifying cancer comprising, determining the level of any of the genes as described hereinabove in a biological sample taken from a tumour, or suspected tumour, wherein tumours expressing elevated levels of DAPK1 and/or reduced levels of NPAS2, NES, CHST7 or EMP1 are predicted as sensitive to treatment with erbB receptor drugs.

According to another aspect of the invention there is provided a method for predicting clinical outcome of treatment with an erbB receptor drug for a mammal, having or suspected of having a tumour, comprising determining the level of any of the genes as described hereinabove in a biological sample taken from the tumour, or suspected tumour, wherein a poor outcome is predicted if.
a) the expression level of DAPK1 or DAPK2 is reduced; and/or
b) the expression level of EMP1 or NES is increased.

According to another aspect of the invention there is provided a method for classifying cancer comprising, determining the level of any of the genes as described hereinabove in a biological sample taken from a tumour, or suspected tumour, wherein tumours expressing elevated levels of DAPK1 or DAPK2 and/or reduced levels of EMP1 or NES are predicted as sensitive to treatment with erbB receptor drugs.

According to another aspect of the invention there is provided a method for treating a disease condition in a mammal having, or suspected of having, a tumour, predicted to be resistant or non responsive to erbB receptor drug treatment based on the level of any of the genes as described hereinabove, comprising: providing a resistance-surmounting quantity of an erbB receptor drug and administering the resistance-surmounting quantity of the erbB receptor drug to the mammal.

In a preferred embodiment the mammal is a primate. In a most preferred embodiment the mammal is a human. In a preferred embodiment the patient is a primate. In a most preferred embodiment the patient is a human.

The term “erbB receptor drug” includes drugs acting upon the erbB family of receptor tyrosine kinases, which include EGFR, erbB2 (HER), erbB3 and erbB4 as described in the background to the invention above. In a preferred embodiment the erbB receptor drug is an erbB receptor tyrosine kinase inhibitor. In a preferred embodiment the erbB receptor drug is an EGFR tyrosine kinase inhibitor.

In a more preferred embodiment the EGF receptor tyrosine kinase inhibitor is selected from gefitinib, Erlotinib (OSI-774, CP-358774), PKI-166, EKB-569, HKI-272 (WAY-177820), lapatinib (GW2016, GW-572016), canertinib (CI-1033, PD183805), ABB788, XL647, BMS 5599626 or any of the compounds as disclosed in WO03/082831, WO05/012290, WO05/026157, WO05/026150, WO05/026156, WO05/028470, WO05/028469, WO2004/006846, WO03082831, WO03/082290 or PCT/GB2005/000237.

In another preferred embodiment the erbB receptor drug is an anti-EGFR antibody such as for example one of cetuximab (C225), matuzumab (EMD-72000), panitumumab (ABX-EGF/rHuMAb-EGFr), MR1-1, IMC-11F8 or EGFRL11.

We contemplate that erbB receptor drugs may be used as monotherapy or in combination with other drugs of the same or different classes. In an especially preferred embodiment the EGF receptor tyrosine kinase inhibitor is gefitinib.

In a preferred embodiment the present invention is particularly suitable for use in predicting the response to the erbB receptor drug as described hereinbefore in those patients or patient population with a tumour which is dependent alone, or in part, on an erbB tyrosine kinase receptor. Such tumours include, for example, non-solid tumours such as leukaemia, multiple myeloma or lymphoma, and also solid tumours, for example bile duct, bone, bladder, brain/CNS, glioblastoma, breast, colorectal, cervical, endometrial, gastric, head and neck, hepatic, lung, muscle, neuronal, oesophageal, ovarian, pancreatic, pleural/peritoneal membranes, prostate, renal skin, testicular, thyroid, uterine and vulval tumours.

In a preferred embodiment the present invention is particularly suitable for identifying a patient with head, neck, pancreatic, glioblastoma, colorectal or breast tumour for drug treatment. In an especially preferred embodiment the present invention also is particularly suitable for identifying those patients with NSCLC, more particularly advanced NSCLC including advanced adenocarcinoma that will respond to treatment with an erbB receptor drug as hereinbefore defined.

The present invention provides advantage in the treatment of tumours such as NSCLC, especially advanced NSCLC by identifying “individual cancer profiles” of NSCLC and so determining which tumours would respond to erbB receptor drug such as gefitinib.

The present invention is particularly useful in the treatment of patients with advanced NSCLC who have failed previous chemotherapy, such as platinum-based chemotherapy. The present invention is also particularly useful in the treatment of patients with locally advanced (stage IIIB) or metastasized (stage IV) NSCLC who have received previous chemotherapy, such as platinum-based chemotherapy. The present invention is also useful in adjuvant therapy or as a first-line therapy.

In a preferred embodiment there is provided a method of selecting a human, having or suspected of having a tumour, for treatment with gefitinib which comprises testing a biological sample, from the mammal for expression of NPAS2, NES, CHST7, DAPK1 and EMP1, whereby to predict an increased likelihood of response to gefitinib.

In a preferred embodiment there is provided a method of selecting a human, having or suspected of having a tumour, for treatment with gefitinib which comprises testing a biological sample, from the mammal for expression of DAPK1, DAPK2, NES and EMP1 whereby to predict an increased likelihood of response to gefitinib.

According to another aspect of the invention there is provided a method of predicting the responsiveness of a patient or patient population with cancer, for example lung cancer, to treatment with chemotherapeutic agents, especially erbB receptor drugs, comprising comparing the differential expression of any of the genes described herein.

In one embodiment the assessment of expression is performed by gene expression profiling using oligonucleotide-based arrays or cDNA-based arrays of any type, particularly where large numbers of genes are analysed simultaneously. In an alternative embodiment, RT-PCR (reverse transcription-Polymerase Chain Reaction), real-time PCR, in-situ hybridisation, Northern blotting, Serial analysis of gene expression (SAGE) for example as described by Velculescu et al Science 270 (5235): 484-487, or differential display or any other method of measuring gene expression at the RNA level could be used. Details of these and other general molecular biology techniques can be found in Current Protocols in Molecular Biology Volumes 1-3, edited by F M Asubel, R Brent and R E Kingston; published by John Wiley, 1998 and Sambrook, J. and Russell, D. W., Molecular Cloning: A Laboratory Manual, the third edition, Cold Spring Harbor Laboratory Press, Cold Spring Harbor, N.Y., 2001.

In another embodiment the assessment of expression is performed by measurement of protein levels encoded by the aforementioned genes. For example, an immunohistochemistry-based assay or application of an alternative proteomics methodology.

In another embodiment the assessment of expression is performed by measurement of activity of the proteins encoded by the aforementioned genes, for example in a bioassay.

In a preferred embodiment the biological sample would have been obtained using a minimally invasive technique to obtain a small sample of tumour, or suspected tumour, from which to determine gene expression profile. Such techniques include, for example tumour biopsy, such as transbronchial biopsy. The profile of gene expression of transbronchial biopsy specimens whose size is about 1 mm may be measured for example using a suitable amplification procedure.

Another aspect of the invention provides a kit for use in a method of predicting the responsiveness of a patient or patient population with a tumour, to treatment with chemotherapeutic agents, especially erbB receptor drugs, comprising a means for measuring the levels of any of the genes as described hereinabove. Preferably the genes are attached to a support material or membrane such as nitrocellulose, or nylon or a plastic film or slide.

In a further preferred embodiment the present invention includes administration of an erbB receptor drug to a mammal selected according the methods described hereinabove.

According to another aspect of the invention there is provided a method of using the results of the methods described above in determining an appropriate dosage of an erbB receptor drug.

In a preferred embodiment the biological sample comprises either a single sample which may be tested for expression of any of the genes as described hereinabove, or multiple samples which may be tested for expression of one or more of the genes as described hereinabove.

The invention is illustrated by the following non-limiting examples in which:

FIG. 1 illustrates a xenograft (A549 cell line) which when grown as a xenograft in athymic mice is sensitive to gefitinib. This involved oral dosing, once daily, at the dose indicated. Y axis=mean tumour volume in cm³; x axis=days after treatment.

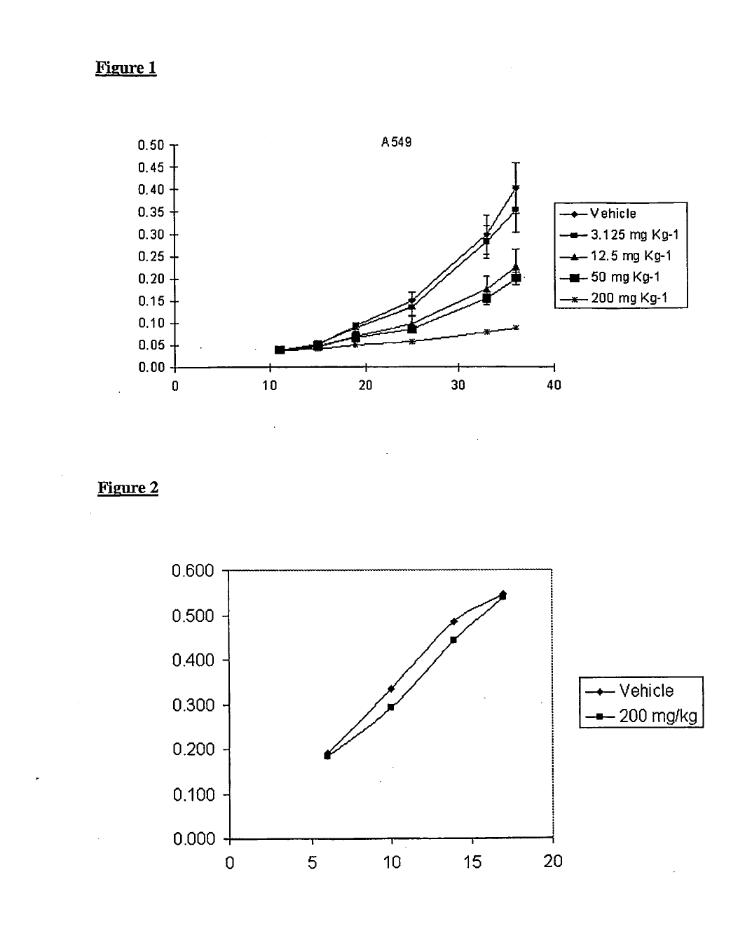

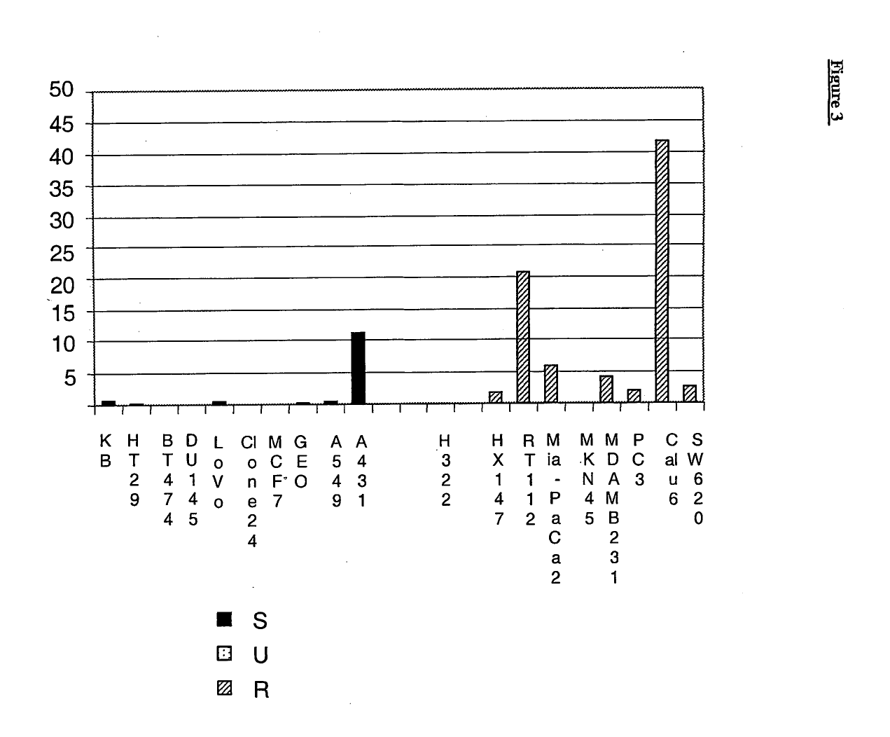

FIG. 2 illustrates a xenograft (MKN45 cell line) which when grown as a xenograft in athymic mice is resistant to gefitinib. This involved oral dosing, once daily, at the dose indicated. Y axis=mean tumour volume in cm³; x axis=days after treatment.

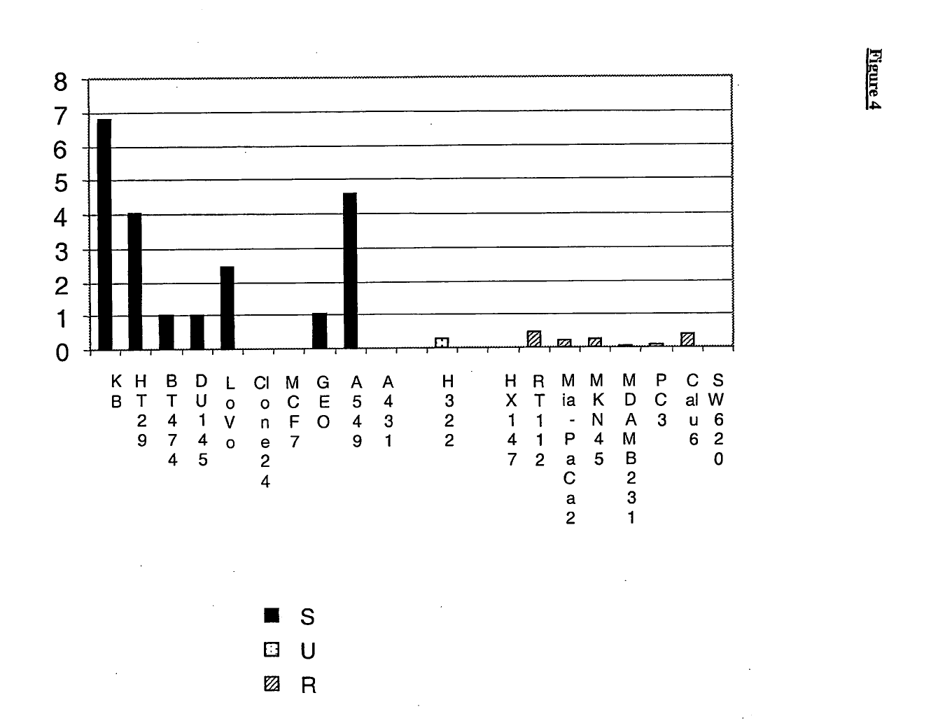

FIGS. 3, 4, 5 and 6 show examples of specific gene expression profiled across a wider panel of gefitinib sensitive and resistant lines, where definition of sensitivity is based on response to gefitinib when grown as a xenograft, to increase confidence that the expression profile of each gene is truly predictive. Iressa sensitivity is based on xenografts data. The cell lines and the tumours from which they are derived are as follows; KB—head and neck, HT29—colon, BT474—breast, DU145—prostate, LoVo—colon, MCF7—breast, GBO—colon, A549—lung, A431—epidermoid, H322—lung, HX147—lung, RT112—bladder, MiaPaCa2—pancreas, MKN45—gastric, MDAMB231—breast, PC3—prostate, Calu6—lung, SW620—colon. The legend key is S=sensitive, U=unknown and R=resistant.

FIG. 3 shows EMP1 basal expression in Cell Culture—wider cell panel (Taqman RT-PCR).

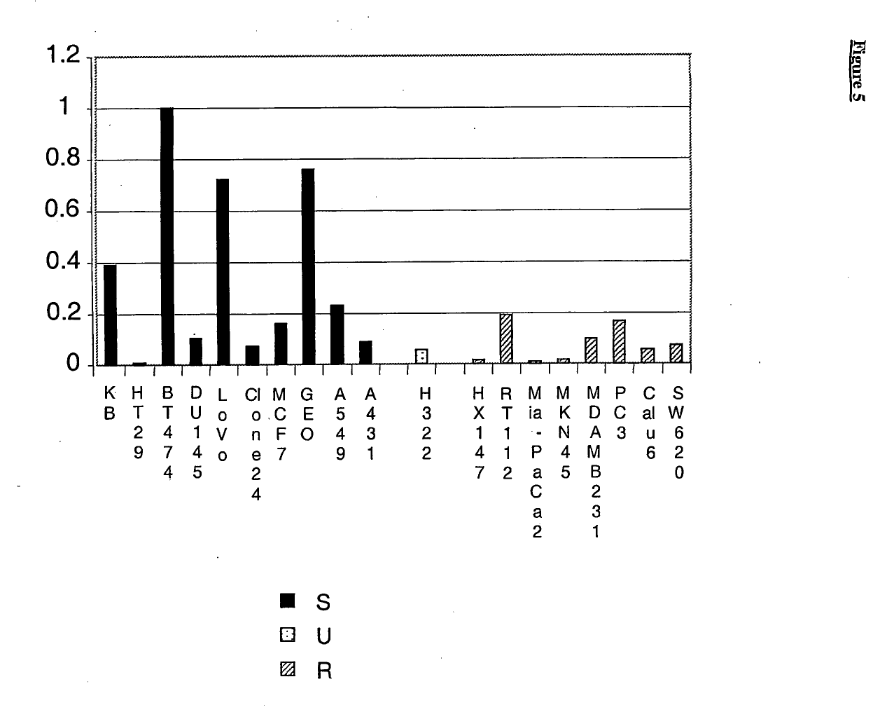

FIG. 4 shows DAPK1 basal expression in Cell Culture—wider cell panel (Taqman RT-PCR).

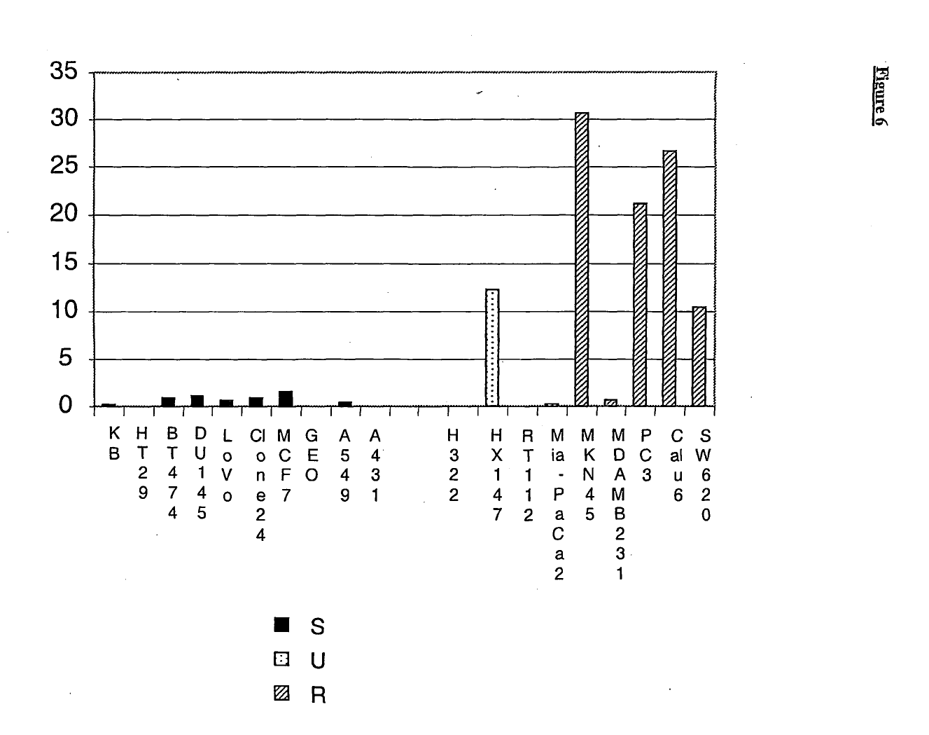

FIG. 5 shows DAPK2 basal expression in Cell Culture—wider cell panel (Taqman RT-PCR).

FIG. 6 shows NES basal expression in Cell Culture—wider cell panel (Taqman RT-PCR).

### EXAMPLE 1

### Gene Expression in Gefitinib Resistant or Sensitive Tumour Cell Lines

### Cell Culture and Xenograft Studies

We identified genes useful to predict response to erbB receptor drugs in the clinic. This is based on studies with gefitinib, but the findings are applicable to erbB receptor drugs in general.

The gene lists have been assembled by comparing tumour cell lines which have been demonstrated to be either sensitive to gefitinib or resistant to gefitinib. This definition is based on the response observed when the tumour cell line is implanted into nude mice and grown as a xenograft. This definition has been used for all the pre-clinical studies described herein.

Initially a small panel of six human tumour cell lines were assembled, three which are sensitive to gefitinib and three which are resistant to gefitinib in the xenograft setting defined above.

The sensitive cell lines were;
1. Lovo (ATCC¹ No. CCL-229)—colon tumour cell line
2. KB (ATCC No. CCL-17)—initially reported as a nasopharyngeal cell line (although more recently reported as Hela derived (cervical carcinoma)
3. HT29 (ATCC No. HTB-38)—colon tumour cell line ¹ATCC=American Type Culture Collection
   The resistant cell lines were;
1. MKN 45 (source—Nottingham University, UK)—gastric tumour cell line
2. Calu 6 (ATCC No. HTB-56)—lung tumour cell line
3. PC3 (ATCC No. CRL-1435)—prostate tumour cell line

The cell lines were grown both in cell culture and as xenografts, RNA prepared and the basal expression profiles determined by measuring RNA expression on the Affymetrix microarray platform. As part of our studies, the term ‘basal’ has been used to indicate constitutive or steady state expression levels (rather than expression levels which are modulated as a consequence of administration of an erbB ligand or gefitinib to the cells). FIG. 1 illustrates the sensitivity of A549 xenografts (used in Example 3 below) to treatment with gefitinib. FIG. 2 illustrates the resistance of MKN45 xenografts to gefitinib. See Example 2 below for analysis of results.

### EXAMPLE 2

### Statistical Analyses of Cell Culture and Xenograft Data Sets

The following statistical analyses were performed separately for cell culture and xenograft data sets. Probe sets were eliminated if their signal was not distinguishable from background noise across all RNA samples in the set. Mixed ANOVA (see for example Scheffe, 1959) was applied separately to each remaining probe set to generate p values. The p values were then used to calculate Q values (Storey). The Q values indicate the expected proportion of genes in a gene list which are not truly differentially expressed but have been falsely discovered (False Discovery Rate or FDR). Q value cut-offs appropriate in the different studies were identified and applied, based on graphical examination of the p value and Q value results, in conjunction with fold change. The final genelists for each study were generated using Q value and fold change (FC) cut-offs. The different genelists were then combined to display an overall list of genes which showed consistent differences in expression profiles between the cell lines in the sensitive and resistant groups.

Further details of the analysis procedures are provided as follows. Fold change (FC) was calculated based on the mean of sensitive cells divided by the mean of resistant cells. To generate gene lists, FC cut-off of two-fold (2×) change in either direction was used in all cases. Furthermore FDR Q values were used to narrow down the lists and obtain the most significant gene changes across sensitive versus resistant cell lines. In the case of cell culture, Q value cut-off is 0.3. In the case of xenograft, Q value cut-off is 0.6. The different cut-offs used reflect the different design and variance values associated with each experiment.

In cell culture studies, lists were obtained based on the above criteria for cells grown either in full serum containing medium or in charcoal stripped serum. In the xenograft study, the same as above was performed for separate sets of tumours harvested at 18 hr intervals. Gene lists contain some redundancy in genes where appropriate to illustrate consistency of results obtained for example with different probe sets.

### EXAMPLE 3

### Identification of Predictive Genes

Genes which have not previously been identified as predictive of erbB receptor drug sensitivity are listed in Table 1. Other genes which we have identified to be optionally used in combination with Table 1 genes are listed in Table 2.

### Key to Tables:

‘Affymetrix ID’—the Affymetrix probe set identifier
‘Sequence’—target sequence relating to the Affymetrix probe set indicated by ‘Affymetrix ID’

“+ if up in sensitive” means that the gene is relatively highly expressed in sensitive cells. (Consequently, absence of a “+” means that the gene is relatively highly expressed in resistant cells).

‘Gene Title’—The current annotation of the gene relating to ‘Affymetrix ID’ based on UniGene 133
‘Gene Symbol’—shorthand synonym for the gene title
‘Locus Link’ & RefSeq Transcript ID’ are provided for gene identification purposes.

Combining genes has the potential to generate an improved diagnostic over genes used in isolation. Collective gene expression profiles (at the RNA and/or protein level) may be more likely to identify patients most likely to benefit from gefitinib rather than the expression level of one gene in isolation.

It may be more practical when developing a pre-treatment response prediction diagnostic to work with a truncated gene list from tables 1 and/or 2. A number of criteria have been used to shorten the gene list to identify those genes which are most predictive of response. Firstly the statistical (p values and Q values or FDR values) can indicate the statistical significance of a gene.

Secondly, the differential expression (fold change) between the sensitive and resistant groups indicates the potential sensitivity of a marker to be used in a diagnostic test (highest fold change between sensitive group and resistant group is preferred).

Thirdly, we have performed RT-PCR based expression profiling across a wider panel of gefitinib sensitive and resistant human tumour cell lines to increase confidence that the expression profile of each gene is truly predictive. FIGS. 3, 4, 5 and 6 show examples of specific gene expression profiled across a wider panel of cell lines as set out below.

The sensitive human tumour cell lines, where definition of sensitivity is based on response to Iressa when grown as a xenograft:
a. BT474 (ATCC No. HTB-20)—breast tumour cell line
b. DU145 (ATCC No. HTB-81)—colon tumour cell line
c. MCF7 (ATCC No. HTB-22, sourced from ICRP (now CR-UK), London), —breast tumour cell line
d. GEO colon tumour cell line. RNA obtained from Fortunato Ciardiello, Cattedra di Oncologia Medica, Dipartimento Medico-Chirurgico di Internistica Clinica e Sperimentale “P. Magrassi e A. Lanzara,” Seconda Universita delgi Studi di Napoli, Via S. Pansini, 5-80131, Naples, Italy.
e. A549 (ATCC No. CCL-185)—lung tumour cell line
f. A431 (ATCC No. CRL-155)—epidermoid cell line

The resistant human tumour cell lines, where definition of sensitivity is based on response to Iressa when grown as a xenograft:

1) HX147—(source: ICRF (now CR-UK), London)—lung tumour cell line

2) RT112—bladder tumour cell line (DSMZ No ACC 418)

3) MiaPac2 (ECACC 85062806, ref. no. 001611) pancreatic tumour cell line

4) MDAMB231 (ATCC No. HTB-26)—breast tumour cell line

5) SW620 (ECACC CCL-227)—colon tumour cell line

### ATCC=Arnerican Type Culture Collection

### DSMZ—Deutsche Sammlung von Mikroorganismen und Zellkulturen GmbH (German Collection of Micro-organisms and Cell Cultures)

### ECACC=European Collection of Cell Cultures

In isolation, each of these genes is reasonably predictive of gefitinib response, but collectively they can be applied to make predictions with a higher level of confidence.

The Affymetrix probe sets identifiers for the genes in the above diagnostic genelists are indicated in Tables 1 and 2. Current Affy IDs are based on Affy U133 chipset. For the avoidance of doubt, the target sequences of the Affymetrix probe sets which identified the listed genes are also provided in Tables 1 and 2.

Without wishing to be bound by theoretical considerations, it is contemplated that the specific sequences used to detect target genes in the Examples may define specific splice variants or sequences in homologous genes. Therefore in one embodiment, a listed gene for use in the method of the invention is defined by the specific sequence used in said Examples. In another embodiment, a gene for use in the method of the invention is not limited by the specific sequence used in these Examples. Indeed the fact that some genes in Tables 1 and 2 have been identified using different sequences (gene “redundancy”) and confirmatory RT-PCR studies (see Example 4) provides evidence that usefulness in the method of the invention is not generally limited to the specific sequences used to measure the target gene.

Note, in the event of a discrepancy in the sequence between Tables 1 and 2 and the Sequence Listing, the sequence as provided in the Tables is preferred.

TABLE 1

| as described in priority application US60/619027 filed on 18/10/2004. | | | | | | | |
|---|---|---|---|---|---|---|---|
| Gene | | Affymetrix | + up in | | | RefSeq | SEQ ID |
| Symbol | Gene Title | ID | sensitive | Sequence | LocusLink | Transcript ID | NO. |
| ACOX2 | ““acyl-Coenzyme | 205364_at | | Gtgcagcatttacagaccctgacgcaatccggagctgaccagcacgaggcttgga | 8309 | NM_003500 | SEQ ID |
| | A oxidase 2, | | | accagaccactgtcatacacctccaggctgctaaggtgcactgctactatgtcactgtg | | | NO: 1 |
| | branched chain/ | | | aagggttttacagaagctctggagaaactagaaaatgaaccagcgattcagcaggt | | | |
| | acyl-Coenzyme A | | | gctcaagcgcctctgtgacctccatgccatacatggaatcttgactaactcgggtgact | | | |
| | oxidase 2, | | | ttctccatgacgccttcctgtctggtgcccaagtggacatggcaagaacagcctacctg | | | |
| | branched chain” | | | gacctgctccgcctgatccggaaggatgccatcctgttaactgatgcttttgacttcacc | | | |
| | | | | gatcagtgtttaaattcagcccttggctgttatgatggaaacgtctacgaacgcctgttcc | | | |
| | | | | agtgggctcagaagtc | | | |
| ACTR2 | ARP2 actin- | 200729_s | | gagcttaagatctggtgtrngttaatgcttctgttattccagaagcattaaggtaaccat | 10097 | NM_005722 SEQ ID | |
| | related protein 2 | _at | | tgccaagtatcattcttgcaaattattcttttatataactgaccagtgcttaataaaacaag | | | NO: 2 |
| | homolog (yeast) | | | caggtacttacaaataattactggcagtaggttataattggtggtttaaaaataacattg | | | |
| | | | | gaatacaggacttgttgccaattgggtaattttcattagttgttttgtttgttttgatttgaaac | | | |
| | | | | ctggaaatacagtaaaatttgactgtttaaaatgttggccaaaaaaatcaagatttaatt | | | |
| | | | | tttttatttgactgaaaaactaatcataactgttaattctcagccatctttgaagcttgaaa | | | |
| | | | | gaagagtctttggtattttgtaaacgttagcagactttcctgccagtgtcagaaaatccta | | | |
| | | | | tttatgaatccttcggtattccttggtatctgaaaaaaataccaaatagtaccatacatg | | | |
| | | | | agttatttctaa | | | |
| APOL1 | “apolipoprotein L | 209546_s | + | agaatagagaggaggcttgaaggaaccagcaatgagaaggccaggaaaagaa | 8542 | NM_003661/ | SEQ ID |
| | 1/apolipoprotein | _at | | agagctgaaaatggagaaagcccaagagttagaacagttggatacaggagaaga | | NM_145343/ | NO: 3 |
| | L, 1” | | | aacagcggctccactacagacccagccccaggttcaatgtcctccgaagaatgaag | | NM_145344 | |
| | | | | tctttccctggtgatggtcccctgccctgtctttccagcatccactctcccttgtcctcctgg | | | |
| | | | | gggcatatctcagtcaggcagcggcttcctgatgatggtcgttggggtggttgtcatgtg | | | |
| | | | | atgggtccctccaggttactaaagggtgcatgtcccctgcttgaacactgaagggcag | | | |
| | | | | gtggt | | | |
| C10orf | chromosome 10 | 209183_s | | aactcatacgtcctgtggtggcattgggagagttcccccatgatgagggccagata | 11067 | NM_007021 | SEQ ID |
| 10 | open reading | _at | | gaatctgtaccactcagtgctaccatccccacccctacaccacttccacacaggggc | | | NO: 4 |
| | frame 10/ | | | ctcatggatggcagggtcccagctgtaggtgagagcagggcactgtccagctgtc | | | |
| | chromosome 10 | | | cactggggaagcaagatgtaaggcccaggtcagggcatctggagtctgaagg | | | |
| | open reading | | | accctagttcctagaggcatctggcagcaagaaggtgaggcatcagggaacggga | | | |
| | frame 10 | | | atcaggctgggactgatcagaggtgaagggacagagagaggagaggaggaaga | | | |
| | | | | ttgagctgggggcaacagccaagctcacctgggcaggtctctgccacctccttgctct | | | |
| | | | | gtgagctgtcagtdaggttattctctttttttgtggctatttttaattgctttggatttgttaaatg | | | |
| | | | | ttttctgtcttctgttaagtgtgttt | | | |
| CALM1 | “calmodulin 1 | 211984_at | | tgaacggctgtgcagtaggcccagcgctgctgtgtctcgtcagaggaatagcttacca | 801 | NM_006888 | SEQ ID |
| | (phosphorylase | | | cgaacccctcagcatactgggaatctcctgaacaacgaatgtaaatttggtcaagt | | | NO: 5 |
| | kinase, delta)” | | | ctactccgttcattcaattattttaagcatttgaattatttattgtatatcctaaatatatttct | | | |
| | | | | cctttggcagtgactagatttccactaatgtgtcttaatctatccctccagctggcagttac | | | |
| | | | | tgtttttttaatcccctgaagttgtcctgtaggagacagaaattctttgctgtctgtatccctt | | | |
| | | | | ggagtaa | | | |
| CALM1 | “calmodulin 1 | 211985_s | | gaggcaaatggatctcgatatttcagatgggcttttgafgcactgttgccaaggaaggc | 801 | NM_006888 | SEQ ID |
| | (phosphorylase | _at | | tttttctgattttttgacaaatgaatttttgcacactttcattggtctttcggcaacttacaca | | | NO: 6 |
| | kinase, delta)” | | | cattgaaaat | | | |
| CD44 | CD44 antigen | 210916_s | | caagttttggtggcacgcagcctggggactctgcctcgtgccgctgagcctggcgca | 960 | NM_000610 | SEQ ID |
| | (homing function | _at | | gatcgatttgaatataacctgccgctttgcaggtgtattccacgtggagaaaaatggtc | | | NO: 7 |
| | and Indian blood | | | gctacagcatctctcggacggaggccgctgacctgcaaggctttcaatagcacctt | | | |
| | group system) | | | gcccacaatggcccagatggagaaagctctgagcatcggatttgagacctgcagttt | | | |
| | | | | gcattgcagtcaacagtcgaagaaggtgtgggcagaagaaaaagctagtgatcaa | | | |
| | | | | cagtggcaatggagctgtggaggacagaaagccaagtggactcaacggagaggc | | | |
| | | | | cagcaagtctcaggaaatggtgcatttggtgaacaaggagtcgtcagaaactccag | | | |
| | | | | accagt | | | |
| CD44 | CD44 antigen | 212063_at | | attgtaaatctttgtgtctcctgaagacttcccttaaaattagctctgagtgaaaaatcaa | 960 | NM_000610 | SEQ ID |
| | (homing function | | | aagagacaaaagacatcttcgaatcatatttcaagcctggtagaattggcttttctag | | | NO: 8 |
| | and Indian blood | | | cagaacctttccaaaagttttatattgagattcataacaacaccaagaattgattttgtag | | | |
| | group system) | | | ccaacattcattcaatactgttatatcagaggagtaggagagaggaaacatttgactta | | | |
| | | | | tctggaaaagcaaaatgtacttaagaataagaataacatggtccattcacctttatgtta | | | |
| | | | | tagatatgtctttgtgtaaatcatttgttttgagttttcaaagaatagcccattgttcattcttgt | | | |
| | | | | gctgtacaatgaccactgttattgttactttgacttttcagagcacaccc | | | |
| CDS2 | CDP- | 212864_at | | ttctatgcatccacaccaaaatcctgcagaatgtaagtaagctctgctttataagatgg | 8760 | NM_003818 | SEQ ID |
| | diacylglycerol | | | gttcaccttcatcgcagactgaaagttcagtttttatttttttncagaaagcacgaaaat | | | NO: 9 |
| | synthase | | | tatttataatagtctggagaaaaaacacactgtaatatttcaagtgtatgcagtagaatg | | | |
| | (phosphatidate | | | tactgtaactgagccctttcccacatgtctaggctccaatgtctgtctcctgtaggtccacctaaa | | | |
| | cytidylyltransferase) | | | ctgtgttttcagggacaatgccatccatgtttgtgctgtagacttgctgctgctgaatcct | | | |
| | 2 | | | ttctggggactttctcatcgggcagggagcagagggcttctcgttcatgcaccctttgcc | | | |
| | | | | tgaacacccatgtagctgctgtgttgtgtatatattactcttaagaggagtgtgtgtgtctgt | | | |
| | | | | gtttgttttaaaagtcacttatttcttacagtgatttcaattgcaccatgacttcttcactaaa | | | |
| | | | | accacaaagtcctgcttaaaactatggaaaacctaacctgattagagccttgac | | | |
| CHST7 | carbohydrate (N- | 208756_at | | ggcaatctgcacactctcagagtctgggacttgacttgctaccaacaactgctgtgca | 56548 | NM_019886 | SEQ ID |
| | acetylglucosamine- | | | attctgctgagcaggaatatcatgagctgttcaataatgacggacgcattggttgagat | | | NO: 10 |
| | 6-O) | | | gaagtttccagtaaggaagtgacagtgcaatgtggatatttatggctgtaaaatagga | | | |
| | sulfotransferase 7/ | | | agagctttagttcccaggctgaacctgccactgctggagccatttcaacaaggcatcc | | | |
| | carbohydrate | | | tcacaacaaagaagagatgtgatttggtaccatttcacaccagcaggtgtctggacg | | | |
| | (N- | | | aaaacatcaatgtgaataagggccaagtgcagtcctgtcttgattaaattacttaataat | | | |
| | acetylglucosamine- | | | attattaaataataataggtctgggcagtattgtttttaacctgactcatccagctgtccttc | | | |
| | 6-O) | | | aaatagctccgtctccctctacccagaactgatttttaaaaagaagtaatttttctccctg | | | |
| | sulfotransferase 7 | | | ggctgggaaaaccctaatgaactgaaacacacttttactttaaaatttttctgtctggcgt | | | |
| | | | | ttttgtaatc | | | |
| COMM | COMM domain | 218048_at | | gaattccctagaaatcctactgggaagtataggcagatctctccctcatataacggatg | 23412 | NM_012071 | SEQ ID |
| D3 | containing 3 | | | tttcttggcgcttggaatatcagataaagaccaatcaacttcataggatgtacagacct | | | NO: 11 |
| | | | | gcatattggtgacctaagtgtacagaacactgattccccatcctatccagagattagtt | | | |
| | | | | ttagttgcagcatggaacaattacaggacttggtggggaaacttaaagatgcttcgaa | | | |
| | | | | aagcctggaaagagcaactcagttgtaacttggggaagttaacgatccgcccgagt | | | |
| | | | | gcagaggaaaaccagaaacgccttgccttcagctgaaccaccgtttgtgcgagctg | | | |
| | | | | gatgtccttttcagtagaaaagaattttccttttgaatttataccattcatcaattttgacactt | | | |
| | | | | taaaaacgtgtgaaagggttaagagggaaagatactgcccaagtatttgaatcgttta | | | |
| | | | | gtagtaactgtccatttatcctat | | | |
| CUL2 | cullin 2/cullin 2 | 203078_at | | tataatacttcagtaaggcctttaaaaaatccacagtgatattattactcctaacaaaaa | 8453 | NM_003591 | SEQ ID |
| | | | | caataattacttagtatcatctaatatgtggttcatatttaaatttgttgttttgagatgggtctt | | | NO: 12 |
| | | | | acaattggtttattcaattgcattttttctaactcgtgtctcaagtgttttaaaaatctactgna | | | |
| | | | | cttataatgacttatataatgtatttctcattttacctttcttccaaaagaggaaataatggc | | | |
| | | | | aaaccatataatattgtacattcactgtcaaaaagcaaacccttgttttgataacttgt | | | |
| DAPK1 | death-associated | 203139_at | + | cctcctccagggtgattttatgatcagtgttgttgctctaggaagacatttttccgtttgctttt | 1612 | NM_004938 | SEQ ID |
| | protein kinase 1/ | | | gttccaatgtcaatgtgaacgtccacatgaaacctacacactgtcatgcttcatcattcc | | | NO: 13 |
| | death-associated | | | ctctcatctcaggtagaaggttgacacagttgtagggttacagagacctatgtaagaat | | | |
| | protein kinase 1 | | | tcagaagacccctgactcatcatttgtggcagtcccttataattggtgcatagcagatgg | | | |
| | | | | tttccacatttagatcctggtttcataacttcctgtacttgaagtctaaaagcagaaaata | | | |
| | | | | aaggaagcaagttttcttccatgattttaaattgtgatcgagttttaaattgataggaggg | | | |
| | | | | aacatgtcctaattcttctgtcctgagaa | | | |
| DNAJC | “DnaJ (Hsp40) | 208499_s | | aggagaggatttgccactgcttttctaaggacgagaagcctgttgaagctattagggttt | 5611 | NM_006260 | SEQ ID |
| 3 | homolog, | | | gttctgaagttttacagatggaacctgacaatgtgaatgccctgaaagatcgagctga | | | NO: 14 |
| | subfamily C, | | | ggcctatttgatagaggaaatgtatgatgaagctattcaggattatgaaactgctcagg | | | |
| | member 3” | | | aacacaatgaaaatgatcagcagattcgagaaggtctagagaaagcacaaagatt | | | |
| | | | | attgaaacagtcgcagaaacgagattattataaaatcttgggagtaaaaagaaatgc | | | |
| | | | | caaaaagcaagaaattattaaagcataccgaaaattagcactgcagtggcaccca | | | |
| | | | | gatgataacttccagaatgaagaagaaaagaaaaaagctgagaaaaagttcattgatat | | | |
| | | | | agcagctgctaaagaagtcctctctgatccagaaatgagaaagaagtttgacgacg | | | |
| | | | | gagaagatcctttggatgcagagagccagcaaggaggcggcggcaaccctttcca | | | |
| | | | | cagaagctggaactcatggcaagggttcaatcccttcagctcaggcggaccatttag | | | |
| | | | | a | | | |
| DPYSL | dihydropyrimidinase- | 201431_s | | tgagggccacgggcttgggtagtggaaagggtgtttgggaaattgttaaatcagttac | 1809 | NM_001387 | SEQ ID |
| 3 | like 3/ | _at | | ccgtagtagagctatttcttgtacttctaagtttctagaagtggaaggattgtagtcatcct | | | NO: 15 |
| | dihydropyrimidinase- | | | gaaaatgggtttacttcaaaatccctagccttgttcttcagactctatactgagagt | | | |
| | like 3 | | | gtcatgtttccacaaagggctgacacctgagcctggattttcactcatccctgagaagc | | | |
| | | | | cctttccagtagggtgggcaattcccaacttccttgccacaagcttcccaggctttctcc | | | |
| | | | | cctggaaaactccagcttgagtcccagatacactcatgggctgccctgggcagccag | | | |
| | | | | cattcatttaagttccctctttgaaaactgggtgggtgttcagttctgtgtctggtgggt | | | |
| | | | | atggacagacagtaatctcctgtgatctgtgctagctgtgaggcagctctggaacgtg | | | |
| | | | | a | | | |
| DUSP4 | dual specificity | 204015_s | | ggctcccagcaagggtaggacgggccgcatgcgggcagaaagttgggactgagc | 1845 | NM_001394/ | SEQ ID |
| | phosphatase 4/ | _at | | agctgggagcaggcgaccgagctccttccccatcatttctccttggccaacgacgag | | NM_057158 | NO: 16 |
| | dual specificity | | | gccagccagaatggcaataaggactccgaatacataataaaagcaaacagaaca | | | |
| | phosphatase 4 | | | ctccaacttagagcaataacggctgccgcagcagccagggaagaccttggtttggttt | | | |
| | | | | atgtgtcagtttcacttttccgatagaaatttcttacctcatttttttaagcagtaaggcttga | | | |
| | | | | agtgatgaaacccacagatcctagcaaatggcccaaccagctttactaaaggggg | | | |
| | | | | aggaagggagggcaaagggatgagaagacaagtttcccagaagtgcctggttctg | | | |
| EIF3S4 | “eukaryotic | 208887_at | | gatacgctggggccatgcagaaggagctggccgagcagctgggcctgtctactgg | 8666 | NM_003755 | SEQ ID |
| | translation | | | cgagaaggagaagctgccgggagagctagagccggtgcaggccacgcagaac | | | NO: 17 |
| | initiation factor 3, | | | aagacagggaagtatgtgccgccgagcctgcgcgacggggccagccgccgcgg | | | |
| | subunit 4 delta, | | | ggagtccatgcagcccaaccgcagagccgacgacaacgccaccatccgtgtcac | | | |
| | 44 kDa/ | | | caactgtcagaggacacgcgtgagaccgacctgcaggagctcttccggcctttcgg | | | |
| | eukaryotic | | | ctccatctcccgcatctacctggctaaggacaagaccactggccaatccaagggctt | | | |
| | translation | | | gccttcatcagcttccaccgccgcgaggatgctgcgcgtgccatttgccggggtgtccg | | | |
| | initiation factor 3 | | | gctttggctacgaccacctcatcctcaacgtcgagtgggccaagccgtccaccaact | | | |
| | subunit 4 delta, | | | aagccagctgccactgtgtactcggtccgggacccttggcgacagaagacagcc | | | |
| | 44 kDa” | | | | | | |
| EIF5A | eukaryotic | 213757_at | | atgtgtcggggagagagcccgcagggaagggtaaagcccannggggcagggcc | 1984 | NM_001970 | SEQ ID |
| | translation | | | ctcccagatgcctgaggagggggcaggtcccctcccctctcctccccat | | | NO: 18 |
| | initiation factor 5A | | | ctaaaggggtttggggagagacacaggcaggcgagggggctggcccagtctgtt | | | |
| | | | | ggggtggtgctcagggtaaagggctatnggcaacaggggaccagaccagggatg | | | |
| | | | | agtggggagggcacaaggaccatttgccagaatccaccg | | | |
| FADS2 | fatty acid | 202218_s | | ctgttgctccaggatgcattctgataggagggggcggcgggctgggccttgtgaca | 9415 | NM_004265 | SEQ ID |
| | desaturase 2/ | _at | | atctgcctttcaccacatggccttgcctcggtggccctgactgtcagggagggccagg | | | NO: 19 |
| | fatty acid | | | gaggcagagcgggagggagtctcaggaggaggcttgccctgaggggctggggag | | | |
| | desaturase 2 | | | ggggtacctcatgaggaccagggtggagcttgagaagaggaggaggtggggctt | | | |
| | | | | ggaggtgcttggtagctgaggggacgggcaagtgagaggggagggagggaagtc | | | |
| | | | | ctgggaggatcctgagctgctgttgcagtctaacccactaatcagttcttagancaggg | | | |
| | | | | gaagggcaggcaccaacaactcagaatgggggctttcggggagggcgcctagtcc | | | |
| | | | | ccccagctctaagcagccaggagggacctgcatctaagcatctgggttgccatggc | | | |
| | | | | aatggcatgccccccagctactgtatgcccccgacccccgcagaggcagaatgaa | | | |
| | | | | cccatagggagctgatcgtaat | | | |
| FLJ124 | hypothetical | 218051_s | | gggaccacctctatagtgatctggcggatctcatgctgcggcacggctggcgcacag | 64943 | NM_022908 | SEQ ID |
| 42 | protein FLJ12442 | _at | | gcgccatcatccccgagctggagcgtgagatccgcatcatcaacacggagcagtac | | | NO: 20 |
| | | | | atgcactgctgacgtggcagcaggcgctcacggggctgctggagcgcatgcaga | | | |
| | | | | cctatcaggacgcggagtcgaggcaggtgcggctgcctggatgaaagagcggca | | | |
| | | | | ggagctgaggtgcatcaccaaggccctgttcatgcgcagttcggcagcatcttccg | | | |
| | | | | caccttccacaaccccacctacttctcaaggcgcctcgtgcgcttctctgacctctacat | | | |
| | | | | ggcctccctcagctgcctgctcaactaccgcgtggacttcaccttctacccacgccgta | | | |
| | | | | cgccgctgcagcacgaggcaccccctctggatggaccagctcttgcaccggctgcatg | | | |
| | | | | aagacccccttccttggtgacatggcccacatccgctgagggcacctttattgtctggg | | | |
| | | | | ac | | | |
| FLJ220 | hypothetical | 213878_at | + | tattcaaacggagcctcccattccaagaaactggaaacccctagtttatgttaaaagg | 79912 | NM_024854 | SEQ ID |
| 28 | protein FLJ22028 | | | ccagtctaaattcttcacttacatctttacagaaaactatattttctctcttccataccag | | | NO: 21 |
| | | | | aaatctaatcagaaactgacttttctcatgttcaactggacctaggggaatatgacag | | | |
| | | | | aaaagcatccataggctttaatatactttttaaaatatataaaactgaaaattaatagc | | | |
| | | | | catttaccctgaaagagttctgcgggactttgtcacttgcatagaatagcatgtgcctc | | | |
| | | | | attgttcagaagattagctttaggtcctattttcaaatacgaaatggtagcataagctgta | | | |
| | | | | aaactgtagtcttctctgcagaaaataaaggccaacaataagaaagcttttgaagga | | | |
| | | | | atcacggaaaacaaatttataaaagaaataactatatgcgcagtaattcttaacacatt | | | |
| | | | | gac | | | |
| FNTB | “famesyltransferase, | 204764_at | | gcaagtcgcgtgattctaccacacctgctactgcctgagcggcctgtccatagccca | 2342 | NM_002028 | SEQ ID |
| | CAAX box, | | | gcacttcggcagcggagcctttgcatgattggcctgggtgtgcccgaaaacgct | | | NO: 22 |
| | beta” | | | ctgcagcccactcacccagtgtacaacattggaccagacaaggtgatccaggccac | | | |
| | | | | tacatactttctacagaagccagcccaggttttgaggagctaaggatgagacatcg | | | |
| | | | | gcagagcctgcaaccgactagaggacctgggtcccggcagctctttgctcacccatc | | | |
| | | | | tccccagtcagacaaggtttatacgtttcaatacatactgcattctgtgctacacaagcc | | | |
| | | | | ttagcctcagtggagctgtggttctcttggtactttcttgaaacaaaccaatggtg | | | |
| | | | | ggtttggagaacacagtggctggttttaaaattctttccacacctgtcaa | | | |
| GPRC5 | “G protein- | 203632_s | | tgatgcacctagcagggcttcaggggttcccactaggatgcagagatgacctctcgc | 51704 | NM_016235 | SEQ ID |
| B | coupled receptor, | _at | | tgcctcacaagcagtgacacctcgggtttccgttgctatggtgaaaattcctggatg | | | NO: 23 |
| | family C, group 5, | | | gaatggatcacatgagggtttcttgttgcttttggagggtgtgggggatattttgttttggtttt | | | |
| | member B/G | | | tctgcaggttccatgaaaacagcccttttccaagcccatttctgtcatggtttccatct | | | |
| | protein-coupled | | | gtcctgagcaagtcattcctttgttatttagcatttcgaacatcggccattcaaagccc | | | |
| | receptor, family | | | ccatgttctctgcactgtttggccagcataacctctagcatcgattcaaagcagagtttta | | | |
| | C, group 5, | | | acctgacggcatggaatgtataaatgagggtgggtccttctgcagatactctaatcact | | | |
| | member B” | | | acattgcttttctataaaactacccataagcctttaacctttaaagaaaaatgaaaaag | | | |
| | | | | gttagtgtttgggggccgggggaggactgaccgcttcataagccagtacgtctgagct | | | |
| | | | | gagta | | | |
| GSPT2 | G1 to S phase | 205541_s | | aagcaattttcttgatgcctctgcaagatactgtgaggagaattgacagcaaaagttca | 23708 | NM_018094 | SEQ ID |
| | transition 2 | _at | | ccacctactctattacgcccattgattgacttttcttcatattttgcaaagagaaatttca | | | NO: 24 |
| | | | | cagcaaaaattcatgttttgtcagctttctcatgttgagatctgttatgtcactgatgaattta | | | |
| | | | | ccctcaagtttccttcctctgtaccactctgcttccttggacaatatcagtaatagctttgta | | | |
| | | | | agtgatgtggacgtaattgcctacagtaatgaaaaattaatgtactttaatttttcatttct | | | |
| | | | | ttaggatatttagaccacccttgcacgcaaaccagagtcagtgtttgtgtg | | | |
| H2AFY | “H2A histone | 218445_at | | cagggatcggaggacgacccgagtcccaagagtggggttttgctttttaaaaggaga | 55506 | NM_018649 | SEQ ID |
| 2 | family, member | | | gaggagggtgatggcaggggagtggagggtggccgggcaggtcctgccggcgc | | | NO: 25 |
| | Y2/H2A histone | | | agggagccctctgcccttcacactctcctccaaaagagcctccatctgtaaggaagc | | | |
| | family, member | | | aggtctccgcgaggggtttctttccatgtgttttcctcctgttgttaaaagaacttttttaaaa | | | |
| | Y2 | | | aaacagactcgttagatttatagcattgacttttacacattcacacaagaaaaa | | | |
| | | | | aatcccaaaattcttaaatcttctgttcctcctttttccaagggaagagggcaaaaag | | | |
| | | | | tggcctgggctctgttggtgtgcgtgttccgtggcggagagaagaaaatgggaaaga | | | |
| | | | | catctcactggtgcttttctcttttgttttagtgccccccgcccccatccctataatatctgta | | | |
| | | | | ac | | | |
| HMGA | high mobility | 208025_s | | gaagcaattgctcatgttggccaaacatggtgcaccgagtgatttccatctctggtaaa | 8091 | NM_003483 | SEQ ID |
| 2 | group AT-hook 2/ | _at | | gttacacttttatttcctgtatgttgtacatcaaaacacactactacttaagtcccagt | | | NO: 26 |
| | high mobility | | | atacctcatttttcatactgaaaaaaaaagcttgtggccaatggaacagtaagaacat | | | |
| | group AT-hook 2/ | | | cataaaatttttatatatatagtttatttttgtgggagataaattttataggactgttctttgctgt | | | |
| | high mobility | | | tgttggtcgcagctaaataagactggacatttaacttttctaccatttctgcaagttaggta | | | |
| | | | | tgtttgccaggagaaaagtatcaagacgtttaactgcagttgactttctcccctgttccttg | | | |
| | | | | agtgtcttctaactttattctttgttctttatgtagaattgctgtctatgattgtactttgaatcgct | | | |
| | | | | tgactgaaaatatttctctagtgtattatcactgtctgttctgcacaataaacataaca | | | |
| | | | | gcctctgtgatccc | | | |
| IER2 | immediate early | 202081_at | | gcgtttccaacctcggagaattccaggcactcccctccgccgcctccgctgacatacttgta | 9592 | NM_004907 | SEQ ID |
| | response 2/ | | | taagcggtcatcgttgcgtcatggggcaggcgtggggagcttcctgtcgccttggctgg | | | NO: 27 |
| | immediate early | | | gtgtgggcctggaggaaggtcctggggcgtgcactcgcctgggcagtggggagga | | | |
| | response 2 | | | gagtggcctgagttacttcacccccgcgtgctgctggttaatgtcccgcgtctctgcacc | | | |
| | | | | ttcgggtgggagcggggactgatctactttcacattctcaagtttttctcatctgcattaga | | | |
| | | | | ggtccccagtaggttcccaggttccagcgtgcccctccctcagacacacggacaca | | | |
| | | | | atcagccgagaagttcctggtctgaatcacgagaatgtggaggggtggggggtgtca | | | |
| | | | | gtggaaaggcataaggctgagctgagaccagttgctggtgaaactgggccaatctg | | | |
| | | | | gggaggggaacatccttgccagggagtttctgagggtctgctttgtttacctttcgtgcg | | | |
| | | | | gggattctttttaactccgtctacctggcgttttgttaga | | | |
| KIAA01 | Vestigial-like | 214004_s | | ccacctgtgaccccgtggtggaggagcatttccgcaggagcctgggcaagaattac | 9686 | NM_014567 | SEQ ID |
| 21 | | _at | | aaggagcccgagccggcacccaactccgtgtccatcacgggctccgtggacgacc | | | NO :28 |
| | | | | actttgccaaagctctgggtgacacgggctccagatcaaagcggccaaggacgga | | | |
| | | | | gcatccagcagccctgagtccgcctctcgcangggccagcccgccagcccctctgc | | | |
| | | | | ccacatggtcagccacagcactccccctctgtggtctcctgaagggagcgcctcctc | | | |
| | | | | caacaacacgtggatctgcatggtttgcctgagctttgaacagtca | | | |
| KIAA09 | KIAA0931 protein | 213407_at | + | attagtctcaagcattcagtgatgtcttcagcatcactataggactgtctagtgtcactt | 23035 | — | SEQ ID |
| 31 | | | | tttacttccttcgggtggaggctttccgactcccaatcatgaaggcaagttaatctttcca | | | NO: 29 |
| | | | | gttagtgacttttgccccatagttggggtaancacttcctagattgagaaaaagcagct | | | |
| | | | | acagcaatcctgctctgtttgcctcatttggtgatcagtcagtcacacataagttccttgt | | | |
| | | | | attctaaatttcatgcacttctcccagatgctatagggttttctctcactgttgccaatggat | | | |
| | | | | gtcatccagacagtgggctcatatcttacggttttgtgc | | | |
| KLHL7 | kelch-like 7 | 220239_at | | agttgatcagagccttccagagtgtggtatgcttttcactgtgtgatgatccttagtggca | 55975 | NM_018846 | SEQ ID |
| | (Drosophila) | | | catgaatgaacgtccagatgtttgtgcagtagcccacccttatctgcaggatacgttcc | | | NO: 30 |
| | | | | aagacccccagtgaatgcctgaaactgcagatagtactgaatcctatatatactgtgtt | | | |
| | | | | ttttatgatacatacatgcctatgatgaagt | | | |
| LAMC2 | “laminin, gamma | 202267_at | | aagagaatgttcctactcacacttcagctgggtcacatccatccctccattcatccttcc | 3918 | NM_005562/ | SEQ ID |
| | 2/laminin, | | | atccatctttccatccattacctccatccatccttccaacatatatttattgagtacctactgt | | NM_018891 | NO: 31 |
| | gamma 2” | | | gtgccaggggctggtgggacagtggtgacatagtctctgccctcatagagttgattgtc | | | |
| | | | | tagtgaggaagacaagcatttttaaaaaataaatttaaacttacaaactttgtttgtcac | | | |
| | | | | aagtggtgtttattgcaataaccgcttggtttgcaacctctttgctcaacagaacatatgtt | | | |
| | | | | gcaagaccctcccatgggggcacttgagttttggcaaggctgacagagctctgggttg | | | |
| | | | | tgcacatttctttgcattccagctgtcactctgtgcctttctacaactgattgcaacagact | | | |
| | | | | gttgagttatgataacaccagtgggaattgctggaggaaccagaggcacttccacctt | | | |
| | | | | ggctgggaagactatggtgctgccttgc | | | |
| MLLT3 | “myeloidllymphoid | 204918_s | + | aaggcattccacaggatcatcatttaaaaaaaaagaattctggtcctgttttctaaaaa | 4300 | NM_004529 | SEQ ID |
| | or mixed- | _at | | aaaaaaactgttgtagaaattcttaatttggatctatttattagtcagagtttcagctttcttc | | | NO: 32 |
| | lineage leukemia | | | agctgccagtgtgttactcatttatcctaaaaatctggaatcagagatttttgtttgttca | | | |
| | (trithorax | | | catatgattctcttagacacttttatatttgaaaaaattaaaatctttctttggggaaaaatt | | | |
| | homolog, | | | cttggttattctgccataacagattatgtattaacttgtagattcagtggttcaatacctgttt | | | |
| | Drosophila); | | | aggcttgctaatatttccagaaggatttcttgtattggtgaaagacggttggggatggg | | | |
| | translocated to, 3/ | | | gggatttttttgttcttgttgtacccttgttttgaaactagaaatctgtcctgtggcatgcaaa | | | |
| | myeloid/lymphoid | | | agaaagcaaattatttttaaaagaaaaaaaccaaagtacttttggtgtcattattccatc | | | |
| | or mixed-lineage | | | ttctcca | | | |
| | leukemia | | | | | | |
| | (trithorax | | | | | | |
| | homolog, | | | | | | |
| | Drosophila); | | | | | | |
| | translocated to | | | | | | |
| | 3” | | | | | | |
| MNAT1 | menage a trois 1 | 203565_s | | ccagccactgcagatagagacatatggaccacatgttcctgagcttgagatgctagg | 4331 | NM_002431 | SEQ ID |
| | (CAK assembly | _at | | aagacttgggtatttaaaccatgtcagagctgcctcaccacaggaccttgctggaggc | | | NO: 33 |
| | factor)/menage | | | tatacttcttctcttgcttgtcacagagcactacaggatgcattcagtgggcttttctggca | | | |
| | a trois 1 (CAK | | | gcccagttaaccatttataagatttggaccttggagctgaaccagggagctagcaaa | | | |
| | assembly factor) | | | agtaaagcagacttataaaattatagctatgtgcagctgcacaacacagtccttccact | | | |
| | | | | agcagctgtgttaa | | | |
| MT1E | metallothionein | 212859_x | + | caccgcgcagagctcagggggtggtgcgcccggcccttctgcggcgcacagccca | 4493 | NM_175617 | SEQ ID |
| | 1E (functional) | _at | | gcccaggaacgcgggcggtgcggactcagcgggccgggtgcaggcgcggagct | | | NO: 34 |
| | | | | gggcctctgcgcccggcccganctccgtctataaanagagcagccagttgcagggc | | | |
| | | | | tcnantctgctttccaactgcctgactgcttgttcgtctcactggtgtgagctccagcatcc | | | |
| | | | | cctttgctcgaaatggaccccaactgctcttgcgccactggntggctcctgcacgtgcg | | | |
| | | | | ccggctcctgcaagtgcaaagagtgcaaatgcacctcctgcaagaagagctgctgtt | | | |
| | | | | cctgctnccccgtggnctgtgccaagtgtgcccagggctgcgtctgcaaaggggcat | | | |
| | | | | cggagaagtgcagctgctgtgcctgatgtgggaacagctcttctcccagatgtaaata | | | |
| | | | | gaacaacctgcacaacctggnatttttttaaaaatacaacactgagccatttgctgcatt | | | |
| | | | | tc | | | |
| NES | nestin | 218678_at | | gcagcaccttaacttacgatctcttgacatacggtttctggctgagaggcctggcccg | 10763 | NM_006617 | SEQ ID |
| | | | | ctaaggtgaaaaggggtgtggcaaaggagcctactccaagaatggaggctgtagg | | | NO: 35 |
| | | | | aatataacctcccaccctgcaaagggaatctcttgcctgctccatctcataggctaagt | | | |
| | | | | cagctgaatcccgatagtactaggtccccttccctccgcatcccgtcagctggaaaag | | | |
| | | | | gcctgtggcccagaggcttctccaaagggagggtgacatgctggcttttgtgcccaag | | | |
| | | | | ctcaccagccctgcgccacctcactgcagtagtgcaccatctcactgcagtagcacg | | | |
| | | | | ccctcctgggccgtctggcctgtggctaatggaggtgacggcactcccatgtgctgact | | | |
| | | | | ccccccatccctgccacgctgtggccctgcctggctagtccctgcctgaataaag | | | |
| NPAS2 | neuronal PAS | 213462_at | | gctacagattcacactttctggcctaaaccctaatgggatgaggcttttcaccccaggc | 4862 | NM_002518/ | SEQ ID |
| | domain protein 2 | | | catgctggtggtgattttttagcccctaaataaaacactggactatttcctgtttacttcatt | | NM_032235 | NO: 36 |
| | | | | gattgcaactacaaaggtggactcaaagcaaagcacaatcatgccagccaacattc | | | |
| | | | | cagaattctgctgagaactccaagtctgtgaggggagaggttttacaagccagacag | | | |
| | | | | gcctgggggactgcagtccccaaggagaccctgccacatgctggccctttgagtga | | | |
| | | | | gaatgctgcatctttctacatatcttcatgagaatactgagaattggattnccttttcaaaa | | | |
| | | | | tgcactttgctttttttgtatgttttgttatgttgagatgtttctaaagaaaagattttatgtaatta | | | |
| | | | | taagatgaagcgtagtgaattgtacagctgttgtaataatgacctatttctatataaaata | | | |
| | | | | aaattgtatggcttatgtgtaaattattttgtatctgagataccagttccttttccc | | | |
| OSGE | O- | 209450_at | | aaaggggatggacgtctcattctcagggatcctgtctttcattgaggatgtagcccatc | 55644 | NM_017807 | SEQ ID |
| P | sialoglycoprotein | | | ggatgctggccacaggcgagtgtactcctgaggatctgtgtttctccctgcaggaaact | | | NO: 37 |
| | endopeptidase | | | gtgtttgcaatgctggtagagatcacagagcgagccatggcacattgtggctcccag | | | |
| | | | | gaggccctcattgtgggaggagtggggtgtaatgtgaggctacaggagatgatggc | | | |
| | | | | aacaatgtgccaggaacgtggagcccggctttttgctacagatgagagattctgtattg | | | |
| | | | | acaatggagcgatgatagcccaggctggctgggagatgtttcgggctggacacagg | | | |
| | | | | accccactcagtgattctggg | | | |
| PCDH | “protocadherin | 209079_x | | cagaaagtctcagcccaggatggggcttcttcaacagggcccctgccctcctgaagc | 5098 | NM_002588/ | SEQ ID |
| GC3 | gamma subfamily | | | ctcagtccttcaccttgccaggtgccgtttctcttccgtgaaggccactgcccaggtccc | | NM_032402/ | NO: 38 |
| | C, 3” | | | cagtgcgccccctagtggccatagcctggttaaagttccccagtgcctcctttgtgcata | | NM_032403 | |
| | | | | gaccttcttctcccacccccttctgcccctgggtccccggccatccagcggggctgcca | | | |
| | | | | gagaaccccagacctgcccttacagtagtgtagcgccccctccctctttcggctggtgt | | | |
| | | | | agaatagccagagtgtagtgcggtgtgcttttacgtgatggcgggtgggcagcgggc | | | |
| | | | | ggcgggctccgcgcagccgtctgtccttgatctgcccgcggcggcccgtgttgtgtttg | | | |
| | | | | tgcgtccacgcgctaaggcgaccccctcccccgtactgacttctcctataagcgctt | | | |
| | | | | ctcttcgcatagcacgtagctcccaccccacctcttcctgtgtctcacgcaagtttta | | | |
| PCDH | “protocadherin | 211066_x | | ggatggggcttcttcaacagggcccctgccccctgaagcctcagtccttcaccttgcc | 5098 | NM002588/ | SEQ ID |
| GC3 | gamma subfamily | _at | | aggtgcgtttctcttccgtgaaggccactgcccaggtccccagtgcgccccctagtg | | NM_032402/ | NO: 39 |
| | C, 3/ | | | gccatagcctggttaaagttcccagtgcctccttgtgcatagaccttcttctcccacccc | | NM_032403 | |
| | protocadhetin | | | cttctgcccctgggtccccggccatccagcggggctgccagagaaccccagacctg | | | |
| | gamma subfamily | | | cccttacagtagtgtagcgccccctccctctttcggctggtgtagaatagccagtagtgt | | | |
| | C, 3” | | | agtgcggtgtgcttttacgtgatggcgggtgggcagcgggcggcgggctccgcgca | | | |
| | | | | gccgtctgtccttgatctgcccgcggcggcccgtgttgtgttttgtgctgtgtccacgcgct | | | |
| | | | | aaggcgaccccctcccccgtactgacttctcctataagcgcttctcttcgcatagtcacg | | | |
| | | | | tagctcccaccccaccctcttcctgtgtctcacgcaagttttatactctaatatttatatggc | | | |
| | | | | tttttttcttcgacaa | | | |
| PCDH | “protocadherin | 215836_s | | gccagctttgggctgagctaacaggaccaatggattaaactggcatttcagtccaag | 5098 | NM_002588/ | SEQ ID |
| GC3 | gamma subfamily | _at | | gaagctcgaagcaggtttaggaccaggtccccttgagaggtcagaggggcctctgt | | NM_032402/ | NO: 40 |
| | C, 3/ | | | gggtgctgggtactccagaggtgccactggtggaagggtcagcggagccccagtgc | | NM_032403 | |
| | protocadherin | | | ctccttgtgcatagaccttcttctcccacccccttctgcccctgggtccccggccatccag | | | |
| | gamma subfamily | | | cggggctgccagagaaccccagacctgcccttacagtagtgtagcgccccctccctc | | | |
| | C, 3” | | | tttcggctggtgtagaatagccagtagtgtagtgcggtgtgcttttacgtgatggcgggt | | | |
| | | | | gggcagcgggcggcgggctccgcgcagccgtctgtccttgatctgcccgcggcggc | | | |
| | | | | ccgtgttgtgttttgtgctgtgtccacgcgctaaggcgaccccctcccccgtactgacttc | | | |
| | | | | tcctataagcgcttctcttcgcatagtcacgtagctcccaccccaccctcttcctgtgtctc | | | |
| | | | | acgcaagtttta | | | |
| PDUM | PDZ and LIM | 203370_s | | tgcacgccctgaagatgacctggcacgtgcactgctttacctgtgctgcctgcaagac | 9260 | NM005451/ | SEQ ID |
| 7 | domain 7 | _at | | gcccatccggaacagggccttctacatggaggagggcgtgccctattgcgagcgag | | NM_203352/ | NO: 41 |
| | (enigma)/PDZ | | | actatgagaagatgtttggcacgaaatgccatggctgtgacttcaagatcgacgctgg | | NM_203353/ | |
| | and LIM domain | | | ggaccgcttcctggaggccctgggcttcagctggcatgacacctgcttcgtctgtgcga | | NM_213636 | |
| | 7 (enigma) | | | tatgtcagatcaacctggaaggaaagaccttctactccaagaaggacaggcctctct | | | |
| | | | | gcaagagccatgccttctctcatgtgtgagccccttctgcccacagctgccgcggtgg | | | |
| | | | | cccctagcctgaggggcctggagtcgtggccctgcatttctgggtagggctggcaat | | | |
| PEX3 | peroxisomal | 203972_s | | tggatccaaacctttattatgccattatatgatgccagatgaagaaactccattagcagt | 8504 | NM_003630 | SEQ ID |
| | biogenesis factor | | | gcaggcctgtggactttctcctcgagacattaccactattaaacttctcaatgaaactag | | | NO: 42 |
| | 3 | | | agacatgttggaaagcccagattttagtacagttttgaatacctgtttaaaccgaggtttt | | | |
| | | | | agtagacttctagacaatatggctgagttctttcgacctactgaacaggacctgcaaca | | | |
| | | | | tggtaactctatgaatagtctttccagtgtcagcctgcctttagctaagataattccaata | | | |
| | | | | gtaaacggacagatcattcagtttgcagtgaaaacctagtcattttgttcaggatctg | | | |
| | | | | ttgacaatggagcaagtgaaagactttgctgctaatgtgtatgaagcttttagtacccct | | | |
| | | | | cagcaactggagaaat | | | |
| PIN1 | protein (peptidyl- | 202927_at | | agccatttgaagacgcctcgtttgcgctgcggacgggggagatgagcgggcccgtgt | 5300 | NM_006221 | SEQ ID |
| | prolyl cis/trans | | | tcacggattccggcatccacatcatcctccgcactgagtgagggtggggagcccag | | | NO: 43 |
| | isomerase) | | | gcctggcctcggggcagggcagggcggctaggccggccagctcccccttgcccgc | | | |
| | NIMA-interacting | | | cagccagtggccgaaccccccactccctgccaccgtcacacagtatttattgttccca | | | |
| | 1/protein | | | caatggctgggagggggcccAtccagattgggggccctggggtccccactccctgtc | | | |
| | (peptidyl-prolyl | | | catccccagttggggctgcgaccgccagattctcccttaaggaattgacttcagcagg | | | |
| | cis/trans | | | ggtgggaggctcccagacccagggcagtgtggtgggaggggtgttccaaagagaa | | | |
| | isomerase) | | | ggcctggtcagcagagccgccccgtgtccccccaggtgctggaggcagactcgag | | | |
| | NIMA-interacting | | | ggccgaattgtttctagttaggccacgctcctctgttcagtcgcaaaggtgaacactcat | | | |
| | 1 | | | gcggcagcc | | | |
| PHKCA | “protein kinase C, | 213093_at | | gattaaacgactgtgtctttgtcacctctgcttaactttaggagtatccattcctgtgattgt | 5578 | NM_002737 | SEQ ID |
| | alpha” | | | agactttgttgatattcttcctggaagaatatcattcttttcttgaagggttggtttactaga | | | NO: 44 |
| | | | | atattcaaaatcaatcatgaaggcagttactattttgagtctaaaggttttctaaaaatta | | | |
| | | | | acctcacatcccttctgttagggtctttcagaatatcttttataaacagaagcatttgaagt | | | |
| | | | | cattgcttttgctacatgatttgtgtgtgtgaaggacataccacgtttaaatcattaattgaa | | | |
| | | | | aaacatcatataagccccaactttgtttggaggaagagacggaggttgaggtttttcctt | | | |
| | | | | ctgtataagcacctactgacaaaatgtagaggccattcaaccgtcaaacaccatttgg | | | |
| | | | | ttatatcgcagaggagacggatgtgtaaattactgcattgctttttttttcagtttgtataacc | | | |
| | | | | tctaatctcgtttgcatgatacgctttgttagaa | | | |
| RIOK3 | RIO kinase 3 | 202129_s | | tgaatgtacgcttgtccatgctgacctcagtgagtataacatgctgtggcatgctggaa | 8780 | NM_003831/ | SEQ ID |
| | (yeast) | _at | | aggtctggttgatcgatgtcagtcagtcagtagaacctacccaccctcacggcctgga | | NM_145906 | NO: 45 |
| | | | | gttcttgttccgggactgcaggaatgtctcgcagtttttccagaaaggaggagtcaagg | | | |
| | | | | aagcccttagtgaacgagaactcttcaatgctgtttcaggcttaaacatcacagcagat | | | |
| | | | | aatgaagctgattttttagctgagatagaagctttggagaaaatgaatgaagatcacgt | | | |
| | | | | tcagaagaatggaaggaaagctgcttcatttttgaaagatgatggagacccaccact | | | |
| | | | | actatatgatgaatagcactaatacccactgcttcagtgttaacacagcagtgattgtc | | | |
| | | | | agctgccaatagcaaatgaagttatgggtgacttgaaataccaaaacctgaggagtg | | | |
| | | | | ggcaatggtgcttctgtg | | | |
| SERPI | “serine (or | 209723_at | + | ttcgccacattggcttgtgttggtcttgaactcctggcctcaagcaatccgcctacctcag | 5272 | NM_004155 | SEQ ID |
| NB9 | cysteine) | | | cctcccaaagtgctaggattacaggcataagccactgagcccagccctagttcagta | | | NO: 46 |
| | proteinase | | | tcttttatgtaaattataaacatctgcaacattatgtatcatatgcagatacttattgcatttct | | | |
| | inhibitor, clade B | | | tttatagtggtgaaagtgttctatgcatttattggctcttgaatttcctcatctatgaattgtca | | | |
| | (ovalbumin), | | | ttcacacacctacttttctgcttcgtttttacatatgtctttgcctattaaagatattatccctct | | | |
| | member 9/ | | | gttttatattttctctcattcttgtattgccttttaa | | | |
| | serine (or | | | | | | |
| | cysteine) | | | | | | |
| | proteinase | | | | | | |
| | inhibitor, clade B | | | | | | |
| | (ovalbumin), | | | | | | |
| | member 9” | | | | | | |
| SIX1 | sine oculis | 205817_at | | ccggaggcaaagagaccgggccgcggaggccaaggaaagggagaacaccga | 6495 | NM_005982 | SEQ ID |
| | homeobox | | | aaacaataactcctcctccaacaagcagaaccaactctctcctctggaagggggca | | | NO: 47 |
| | homolog 1 | | | agccgctcatgtccagctcagaagaggaattctcacctccccaaagtccagaccag | | | |
| | (Drosophila)/ | | | aactcggtccttctgctgcagggcaatatgggccacgccaggagctcaaactattctc | | | |
| | sine oculis | | | tcccgggcttaacagcctcgcagcccagtcacggcctgcagacccaccagcatcag | | | |
| | homeobox | | | ctccaagacctgctcggcccctcacctccagtctggtggacttggggtcctaagt | | | |
| | homolog 1 | | | ggggagggactggggcctcgaagggattcctggagcagcaaccactgcagcgact | | | |
| | (Drosophila) | | | agggacacttgtaaatagaaatcaggaacatttttgcagcttgtttctggagttgtttgcg | | | |
| | | | | cataaaggaatggtggactttcacaaatatctttttaaaaatcaaaaccaacagcgat | | | |
| | | | | ctcaagcttaa | | | |
| SLCO3 | “solute carrier | 219229_at | | ggctgagcaccagtgagttctttgcctctactctgaccctagacaacctggggaggga | 28232 | NM_013272 | SEQ ID |
| A1 | organic anion | | | ccctggcccgcaaccagacacataggacaaagtttatctataacctggaagacc | | | NO: 48 |
| | transporter | | | atgagtgggtgaaaacatggagtccgttttatagtgactaaaggagggctgaactct | | | |
| | family, member | | | gtattagtaatccaagggtcatttttttcttaaaaaaagaaaaaaaggttccaaaaaaa | | | |
| | 3A1/solute | | | accaaaactcagtacacacacacaggcacagatgcacacacacgcagacagac | | | |
| | carrier organic | | | acaccgactttgtcctttttctcagcatcagagccagacaggattcagaataaggaga | | | |
| | anion transporter | | | gaatgacatcgtgcggcagggtcctggaggccactcgcgcggctgggccacagag | | | |
| | family, member | | | tctactttgaaggcacctcatggttttcaggatgctgacagctgcaagcaacaggcact | | | |
| | 3A1” | | | gccaaattcagggaacagtggtggccagcttggaggatggac | | | |
| SPINK1 | “serine protease | 206239_s | + | gagacgtggtaagtgcggtgcagttttcaactgacctctggacgcagaacttcagcca | 6690 | NM_003122 | SEQ ID |
| 1 | inhibitor, Kazal | | | tgaaggtaacaggcatctttcttctcagtgccttggccctgttgagtctatctggtaacact | | | NO: 49 |
| | type 1/serine | | | ggagctgactccctgggaagagaggccaaatgttacaatgaacttaatggatgcac | | | |
| | protease inhibitor, | | | caagatatatgaccctgtctgtgggactgatggaaatacttatcccaatgaatgcgtgtt | | | |
| | Kazal type 1” | | | atgttttgaaggtcggaaacgccagacttctatcctcattcaaaaatctgggccttgctg | | | |
| | | | | agaaccaaggttttgaaatcccatcaggtcaccgc | | | |
| SPINK | “serine | 205185_at | + | agccatcccatgttagagcttctcaagaggaagacagcccagactctttcagttctctg | 11005 | NM_006846 | SEQ ID |
| 5 | protease | | | gatctgagatgtgcaaagactaccgagtattgcccaggataggctatctttgtccaaa | | | NO: 50 |
| | inhibitor, Kazal | | | ggatttaaagcctgtctgtggtgacgatggccaaacctacaacaatccttgcatgctct | | | |
| | type 5/serine | | | gtcatgaaaacctgatacgccaaacaaatacacacatccgcagtacagggaagtgt | | | |
| | protease inhibitor, | | | gaggagagcagcaccccaggaaccaccgcagccagcatgcccccgtctgacga | | | |
| | Kazal type 5” | | | atgacaggaagattgttgaaagccatgagggaaaaaataaaccccagttctgaatc | | | |
| | | | | acctaccttcaccatctgtatatacaaagaattcttcggagcttgtcttatttgctatagaa | | | |
| | | | | aacaatacagagcttttgggaatggaatcactgattttcagtcttttccatttctttcctccta | | | |
| | | | | gaatctgtgatctgagggtataaagacatttccaccaagtttgagccctcaaaatgtcc | | | |
| | | | | tgattacaatgctgtctgtcc | | | |
| STC2 | stanniocalcin | 2203438_at | | gtccacattcctgcaagcattgattgagacatttgcacaatctaaaatgtaagcaaagt | 8614 | NM_003714 | SEQ ID |
| | | | | agtcattaaaaatacaccctctacttgggctttatactgcatacaaatttactcatgagcc | | | NO: 51 |
| | | | | ttcctttgaggaaggatgtggatctccaaataaagatttagtgtttattttgagctctgcatc | | | |
| | | | | ttaacaagatgatctgaacacctctcctttgtatcaataaatagccctgttattctgaagt | | | |
| | | | | gagaggaccaagtatagtaaaatgctgacatctaaaactaaataaatagaaaaca | | | |
| | | | | ccaggccagaactatagtcatactcacacaaagggagaaatttaaactcgaaccaa | | | |
| | | | | gcaaaaggcttcacggaaatagcatggaaaaacaatgcttccagtggccacttccta | | | |
| | | | | aggaggaacaaccccgtctgatctcagaattggcaccacgtgagcttgctaagtgat | | | |
| | | | | aatatctgtttctactacggatttaggcaacaggacctgtacattgtcacattgcat | | | |
| TAZ | transcriptional co- | 202132_at | | tgggggacttatttgttggggatcttaaataagattccttttgatctaccggaatatacatg | 25937 | NM_015472 | SEQ ID |
| | activator with | | | tacagagacattggatcatgttggaaagaaggcaagtgaaaaggtcagagatgaa | | | NO: 52 |
| | PDZ-binding | | | gtagcgaagttatggaatatcgtggaaaggatactagttgtgaaatggaaagagaca | | | |
| | motif (TAZ) | | | agttatagtaccccaaaagcaaaacaagcaggagatgcaagagatgccccaaaa | | | |
| | | | | ggacaaagcaacaattttctgttgccacctttataccggaagactctgttgtagaagaa | | | |
| | | | | aagaaggctttggtgcaccttatgtgggaggaggaggggcagggcatgctgatgct | | | |
| | | | | gagcgtacaggcagacaagagcgtagcctgctgttgcctccatcactatgaaatgac | | | |
| | | | | ttattttacctgaaggacccatggtttatgttcctctaattcctttcactctccctaagccctct | | | |
| | | | | gagagagatg | | | |
| TCEA2 | “transcription | 203919_at | | gcctgtcggctcagatcgaggaatgcatcttccgggacgttggaaacacagacatga | 6919 | NM_003195/ | SEQ ID |
| | elongation factor | | | agtataagaaccgtgtacggagtcgtatctccaacctgaaggatgccaagaaccctg | | NM_198723 | NO: 53 |
| | A (SII), 2/ | | | acctgcggcggaatgtgctgtgtggggccataacaccccagcagatcgctgtgatga | | | |
| | transcription | | | cctcagaggagatggccagtgatgagctgaaggagatccgtaaggccatgaccaa | | | |
| | elongation factor | | | ggaggccatccgagagcaccagatggcccgcactggcggcacgcagacagacct | | | |
| | A (SII), 2” | | | gttcacctgcggcaagtgcaggaaaaagaactgcacctacacacaggtgcagaoc | | | |
| | | | | cgcagctctgatgagcccatgaccacctttgttgtctgcaacgagtgtggaaaccgct | | | |
| | | | | ggaagttctgctgacccctcgtgtagatgtgctgcagccttgggccctccccggccca | | | |
| | | | | cgtcctccgttgacacagcttctctggagaccctagaaggcggcatgtcc. | | | |
| TNNC1 | “troponin C, slow/ | 209904_at | + | tggatgacatctacaaggctgcggtagagcagctgacagaagagcagaaaaatga | 7134 | NM_003280 | SEQ ID |
| | troponin C, | | | gttcaaggcagccttcgacatcttcgtgctgggcgctgaggatggctgcatcagcacc | | | NO: 54 |
| | slow” | | | aaggagctgggcaaggtgatgaggatgctgggccagaaccccacccctgaggag | | | |
| | | | | ctgcaggagatgatcgatgaggtggacgaggacggcagcggcacggtggactttg | | | |
| | | | | atgagttcctggtcatgatggttcggtgcatgaaggacgacagcaaagggaaatctg | | | |
| | | | | aggagctgtctgacctcttccgcatgtttgacaaaaatgctgatggctacatcgacctg | | | |
| | | | | gatgagctgaagataatgctgcaggctacaggcgagaccatcacggaggacgac | | | |
| | | | | atcgaggagctcatgaaggacggagacaagaacaacgacggccgcatcgactat | | | |
| | | | | gatgagt | | | |
| TRPM2 | “transient | 205708_s | | acccttggccatcaggcgaggggctgggcctgtgcagctgggcccttggccagagt | 7226 | NM_00100118 | SEQ ID |
| | receptor potential | _at | | ccactcccttcctggctgtgtcaccccgagcagctcatccaccatggaggtcattggcc | | 8/ | NO: 55 |
| | cation channel, | | | tgaggcaagttccccggagagtcgggntcccctgtggccccctcaggcctatgtctgt | | NM_003307 | |
| | subfamily M, | | | gaggaaggggccctgccactctccccaagagggcctccatgtttcgaggtgcctcaa | | | |
| | member 2” | | | catggagccttgcctggcctgggctaggggcactgtctgaactcctgactgtcaggat | | | |
| | | | | | | aaactccgtgggggtacaggagcccagacaaagcccaggcctgtcaagagacgc | |
| | | | | agagggcccctgccagggttggccccagggaccctgggacgaggctgcagaagc | | | |
| | | | | tctccctccctactccctgggagccacgtgctggccatgtggccagggacggcatga | | | |
| | | | | gcaggaggcggggacgtgggggccttctggtttggtgtcaacagc | | | |
| VAMP4 | vesicle- | 213480_at | | gaagccacaaagatgccacatgttagtatatcagtgagaggtgactccacagtgctc | 8674 | NM_003762/ | SEQ ID |
| | associated | | | tctggagaagcaatatgagtgactgaagagtggggccttttgcttttgcctggatatag | | NM_201994 | NO: 56 |
| | membrane | | | gggtgctcttctactgtaattgggtgtggaaaaactctggctttatggtattccattaggttc | | | |
| | protein 4 | | | ttttcattaaagtagtcttaaaatcaaagtatccaatattttaaagccacaaagtagatt | | | |
| | | | | acataattagcagagattttagtcagtaaaatgttagaaatcaaactataagaaaattc | | | |
| | | | | aagtttattttgtgcttgggtatatgtcattattttaaattccacactcccttatttaatca | | | |
| | | | | ctttggtaagtgcctttgatgttttgaaatgtatagtgggagatgagcaaatgtaaatgtc | | | |
| | | | | atgtgccctgttccctagcttctcaattcctcataaccatttttaccagtgttgcaaagttta | | | |
| | | | | gacctttgtgttaatatcagaagtgtatttgtagcccctccatagtgaacaatga | | | |
| ZNF31 | zinc finger protein | 200868_s | | gccgaagaagctgtggggtgtgtcgcagcgctctggcacctggcgtccgagc | 55905 | NM_018683 | SEQ ID |
| 3 | 313 | _at | | cgtggagctcgagcggcagatcgagagcaagagacttcttgccatggctgccgta | | | NO: 57 |
| | | | | agaatttcttcctgtccaagatccggtcccacgtggctacttgttccaaataccagaatt | | | |
| | | | | acatcatggaaggtgtgaaggccaccattaaggatgcatctcttcagccaaggaatg | | | |
| | | | | tcccaaaccgttacacctttccttgtccttactgtcctgagaagaactttgatcaggaag | | | |
| | | | | gacttgtggaacactgcaaattattccatagcacggataccaaatctgtggtttgtccg | | | |
| | | | | atatgtgcctcgatgccctggggagaccccaactaccgcagcgccaacttcagaga | | | |
| | | | | gcacatccagcgccggcaccggttttcttatgacacttttgtggattatgatgttgatgaa | | | |
| | | | | gaggacatgatgaatcaggtgttgcagcgctccatcatcgaccagtgagcagagtcc | | | |
| | | | | gtgcttgctatc | | | |

TABLE 2

| as described in priority application US60/619027 ified on 18/10/2004. | | | | | | | |
|---|---|---|---|---|---|---|---|
| | | | + up In | | | RefSeq | |
| Gene | | | sensi- | | Locus | Transcript | SEQ ID |
| Symbol | Gene Title | Affymetrix ID | tive | Sequence | Link | ID | NO. |
| AGXT2L1 | alanine-glyoxylate | 221008_s_at | + | gctgaaagaagcccacatagaactgcttagggacagcaccactgactccaaa | 64850 | NM_031279 | SEQ ID |
| | aminotransferase | | | gaaaatcccagcagaaagagaaatggaatgtgcacggatacacattcactgct | | | NO: 58 |
| | 2-like 1/alanine- | | | cagtaagaggctcaagacatgactgatttgcattttaaagcaagatgcgatgtcc | | | |
| | glyoxylate | | | agagttacagagaatgagtagatgtgtctcatcggttaatagctctattatacctct | | | |
| | aminotransferase 2-like 1/ | | | aaaggtggaattgtcagtttagattcataaatgaaaaggtaaatgagtaatcaga | | | |
| | alanine-glyoxylate | | | ataaaccaagtgataatcaaaccatgtcaagattattagttcagactctagcctgtt | | | |
| | aminotransferase 2-like 1 | | | aattttcttagttgatttctgaagctacctgatttattctattaaattgtaagcttgcaaa | | | |
| | | | | ctcaaaataaattggcagatttacctctcatgttttaatgtgtcaaattagagagca | | | |
| | | | | aagtataacaggtgccttcacttttgagactt | | | |
| AKAP12 | A kinase (PRKA) anchor | 210517_s_at | | gtgccatagtgcaggcttggggagctttaagcctcagttatataacccacgaaaa | 9590 | NM_005100/ | SEQ ID |
| | protein (gravin) 12A | | | acagagcctcctagatgtaacattcctgatcaaggtacaattctttaaaattcacta | | NM_144497 | NO: 59 |
| | kinase (PRKA) anchor | | | atgattgaggtccatatttagtggtactctgaaattggtcactttcctattacacgga | | | |
| | protein (gravin) 12 | | | gtgtgctaaaactaaaaagcattttgaaacatacagaatgttctattgtcattggga | | | |
| | | | | aatttttctttctaacccagtggaggttagaaagaagttatattctggtagcaaatta | | | |
| | | | | actttacatcctttttcctacttgttatggttgtttggaccgataagtgtgcttaatcctga | | | |
| | | | | ggcaaagtagtgaatatgttttatatgttatgaagaaaagaattgttgtaagtttttga | | | |
| | | | | ttctactcttatatgctggactgcattcacacatggcatgaaataagtcaggttctta | | | |
| | | | | caaatggtattttgatagatactggattgtgtttgtgccatatttgtgccatt | | | |
| ANXA6 | annexin A6/annexin A6 | 200982_s_at | | gggatgcatttgtggccattgttcaaagtgtcaagaacaagcctctcttctttgccg | 309 | NM_001155/ | SEQ ID |
| | | | | acaaacttacaaatccatgaagggtgctggcacagatgagaagactctgacc | | NM_004033 | NO: 60 |
| | | | | aggatcatggtatcccgcagtgagattgacctgctcaacatccggagggaattc | | | |
| | | | | attgagaaatatgacaagtctctccaccaagccattgagggtgacacctccgga | | | |
| | | | | gacttcctgaaggccttgctggctctctgtggtggtgaggactagggccacagctt | | | |
| | | | | tggcgggcacttctgccaagaaatggttatcagcaccagccgccatggccaag | | | |
| | | | | cctgattgttccagctccagagactaaggaaggggcaggggtggggggaggg | | | |
| | | | | gttgggttgggctcttatcttcatggagcttaggaaacgctcccactcccacgggc | | | |
| | | | | catcgagggccagcacggctgagcggtgaaaaaccgtagccatagatcctgt | | | |
| | | | | cc | | | |
| AREG | amphiregulin | 205239_at | + | atttcaaaatttctgcattcacggagaatgcaaatatatagagcacctggaagca | 374 | NM_001657 | SEQ ID |
| | (schwannoma-derived | | | gtaacatgcaaatgtcagcaagaatatttcggtgaacggtgtggggaaaagtcc | | | NO: 61 |
| | growth factor)/ | | | atgaaaactcacagcatgattgacagtagtttatcaaaaattgcattagcagcca | | | |
| | amphiregulin | | | tagctgcctttatgtctgctgtgatcctcacagctgttgctgttattacagtccagctta | | | |
| | (schwannoma-derived | | | gaagacaatacgtcaggaaatatgaaggagaagctgaggaacgaaagaaa | | | |
| | growth factor) | | | cttcgacaagagaatggaaatgtacatgctatagcataactgaagataaaatta | | | |
| | | | | caggatatcacattggagtcactgccaagtcatagccataaatgatgagtcggtc | | | |
| | | | | ctctttccagtggatcataagacaatggaccctttttgttatgatggttttaaactttca | | | |
| | | | | attgtcactttttatgctatttctgtata | | | |
| BCAT1 | “branched chain | 214452_at | + | gacaacagccctggaggggaacagagtgagagagatgtttngctctggtaca | 586 | NM_005504 | SEQ ID |
| | aminotransferase 1, | | | gcctggttgtttgcccagtttctgatatactgtacaaaggcgagacaatacacatt | | | NO: 62 |
| | cytosolic/branched chain | | | ccaactatggagaatggtcctaagctggcaagccgcatcttgagcaaattaact | | | |
| | aminotransferase 1, | | | gatatccagtatggaagagaagagagcgactggacaattgtgctatcctgaatg | | | |
| | cytosolic” | | | gaaaatagaggatacaatggaaaatagaggataccaactgtatgctactggga | | | |
| | | | | cagactgttgcatttgaattgtgatagatttctttggctacctgtgcataatgtagtttgt | | | |
| | | | | agtatcaatgtgttacaagagtgattgtttcttcatgccagagaaaatgaattgcaa | | | |
| | | | | tcatcaaatggtgtttcataacttggtagtagtaacttaccttaccttaccnanaaaa | | | |
| | | | | atattaatgtaagccatataacatgggattttcctcaannannnnannnnnncct | | | |
| | | | | tttgtacttcactcagatacta | | | |
| BCL3 | B-cell CLL/lymphoma 3/ | 204908_s_at | + | gggcagatcttggactcatgaggaggggcccccctgcccagaggggtcaacc | 602 | NM_005178 | SEQ ID |
| | B-cell CLL/lymphoma 3 | | | cttctggaaactgtgaagatctgacttcgcccccccccccccccatcttcgggac | | | NO: 63 |
| | | | | caggatttgcacagaagcacatgcacctacccatacaccccctcttctgagcgtc | | | |
| | | | | cctgttcccccatctcgctccctcccaggactctgaccccagcattctcaggcacc | | | |
| | | | | agtccctgtccggaatgccacccacatcttccatttccatgtcccctcccagagct | | | |
| | | | | ggtggacccagggaacagccactcccctccactctctaccagataactgagga | | | |
| | | | | ggggagaggtgggccgtaacgggcacggatcacgatgtaaattatt | | | |
| BST2 | bone marrow stromal cell | 201641_at | + | agcttcaggacgcgtctgcagaggtggagcgactgagaagagaaaaccagg | 684 | NM_004335 | SEQ ID |
| | antigen 2/bone marrow | | | tcttaagcgtgagaatcgcggacaagaagtactaccccagctcccaggactcc | | | NO: 64 |
| | stromal cell antigen 2 | | | agctccgctgcggcgccccagctgctgattgtgctgctgggcctcagcgctctgct | | | |
| | | | | gcagtgagatcccaggaagctggcacatcttggaaggtccgtcctgctcggcttt | | | |
| | | | | tcgcttgaacattcccttgatctcatcagttctgagcgggtcatggggcaacacgg | | | |
| | | | | ttagcggggagagcacggggtagccggagaagggcctctggagcaggtctg | | | |
| | | | | gaggggccatggggcagtcctgggtgtggggacacagtcgggttgacccagg | | | |
| | | | | gctgtctccctccagagcctccctccggataatgagtcccccctcttgtctcccac | | | |
| | | | | cctgagattgggcatggggtgcggtgtggggggcatgtgctgcctgttgttatggg | | | |
| | | | | ttttttttgcggggggggttgcttttttctggggtctttgagctccaaa | | | |
| C9orf16 | chromosome 9 open | 204480_s_at | + | catgctggaccagatcaactcctgtctggaccacctggaggagaagaatgacc | 79095 | NM_024112 | SEQ ID |
| | reading frame 16 | | | acctccacgcccgcctccaggagctgctggagtccaaccggcagacacgcct | | | NO: 65 |
| | | | | ggagttccagcagcagctcggggaggcccccagtgatgccagcccctaggct | | | |
| | | | | ccaagagcccccaaccgggacccaaccctgcctccctgggctaggctctggc | | | |
| | | | | ctgggcactcaccccctggcttagacaccttctcaagggctggccttcagggacc | | | |
| | | | | cctggtgggtctgcctgcctgggccacccttcctgcctgggcctccccttggccta | | | |
| | | | | cctgggccagcccccaccacctggcatgccctcctggggccaagagtgggcct | | | |
| | | | | gcaacccacccacttgcctgcccacccaactcctgggcgctccccactctgccc | | | |
| | | | | aggccttgagtgtccacattaaatg | | | |
| CAPZA2 | “capping protein (actin | 201237_at | | cacttaccagtgagcatatatattttaaaatactttctttggatattgtaattcttaactg | 830 | NM_006136 | SEQ ID |
| | filament) muscle Z-line, | | | gttgtaaattagaaaagctgggattacatatggtgtgcggttacagtctaaattttttc | | | NO: 66 |
| | alpha 2” | | | atcctcctatgcatcataagcatgtttgtaatattttcaaaaatagttctactgatgcta | | | |
| | | | | caggaatttcaagcctgtggtgaatgttagtatttaccatagggagtgaagtggag | | | |
| | | | | ttatggtttcaaatagagtattgctgattatacttgagtggaatcctttcctcacgt | | | |
| | | | | actcccacagacgtctgggcctggaaa | | | |
| CCND1 | cyclin D1 (PRAD1: | 208711_s_at | | ggcggaggagaacaaacagatcatccgcaaacacgcgcagaccttcgttgcc | 595 | NM_001758/ | SEQ ID |
| | parathyroid adenomatosis | | | ctctgtgccacagatgtgaagttcatttccaatccgccctccatggtggcagcggg | | NM_053056 | NO: 67 |
| | 1) | | | gagcgtggtggccgcagtgcaaggcctgaacctgaggagccccaacaacttc | | | |
| | | | | ctgtcctactaccgcclcacacgcttcctctccagagtgatcaagtgtgacccgga | | | |
| | | | | ctgcctccgggcctgccaggagcagatcgaagccctgctggagtcaagcctgc | | | |
| | | | | gccaggcccagcagaacatggaccccaaggccgccgaggaggaggaaga | | | |
| | | | | ggaggaggaggaggtggacctggcttgcacacccaccgacgtgcgggacgt | | | |
| | | | | ggacatctgagggcgccaggcaggcgggcgccaccgccacccgcagcgag | | | |
| | | | | ggcggagccggccccaggtgctcccctgacagtccctcctctccggagcattttg | | | |
| | | | | ataccagaagggaaagcttcattctccttgtttgttggttgttttttcctttgctctttcccc | | | |
| | | | | cttccatctctgacttaagcaaaa | | | |
| CCND1 | cyclin D1 (PRAD1: | 208712_at | | gttttgggtatgtttaatctgttatgtactagtgttctgtttgttttgttttgttaattacacc | 595 | NM_001758/ | SEQ ID |
| | parathyroid adenomatosis | | | ataatgctaatttaaagagactcccaaatctccaatgaagccagctcacagtgctgt | | NM_053056 | NO: 68 |
| | 1)/ cyclin D1 (PRAD1: | | | gtgccccggtcatctagcaagctgccgaaccaaaagaatttgcaccccgctgc | | | |
| | parathyroid adenomatosis | | | gggcccacgtggttggggccctgccctggcagggtcatcctgtgctcggaggcc | | | |
| | 1) | | | atctcgggcacaggcccaccccgccccacccctccagaacacggctcacgctt | | | |
| | | | | acctcaaccatcctggctgcggcgtctgtctgaaccacgcgggggccttgaggg | | | |
| | | | | acgctttgtctgtcgtgatggggcaagggcacaagtcctggatgttgtgtgtatcg | | | |
| | | | | agaggccaaaggctggtggcaagtgcacggggcacagcggagtctgtcctgt | | | |
| | | | | gacgcgcaagtctgagggtctgggcggcg | | | |
| CDH1 | “cadhenn 1, type 1, E- | 201130_s_at | | aattcctgccattctggggattcttggaggaattcttgctttgctaattctgattctgctg | 999 | NM_004360 | SEQ ID |
| | cadherin (epithelial)” | | | ctcttgctgtttcttcggaggagagcggtggtcaaagagcccttactgcccccaga | | | NO: 69 |
| | | | | ggatgacacccgggacaacgtttattactatgatgaagaaggaggcggagaa | | | |
| | | | | gaggaccaggactttgacttgagccagctgcacaggggcctggacgctcggcc | | | |
| | | | | tgaagtgactcgtaacgacgttgcaccaaccctcatgagtgtcccccggtatcttc | | | |
| | | | | cccgccctgccaatcccgatgaaattggaaattttattgatgaaaatctgaaagc | | | |
| | | | | ggctgatactgacccacagccccgccttatgattctctgctcgtgtttgactatga | | | |
| | | | | aggaagcggttccgaagctgcagtctgagctccctgaactcctcagagtcaga | | | |
| | | | | caaagaccaggactatgactacttgaacgaatggggcaatccgttcaagaagc | | | |
| | | | | tggctgacatgtacggaggcg | | | |
| CDKN2A | “cyclin-dependent kinase | 207039_at | + | cttttcactgtgttggagttttctggagtgagcactcacgccctaagcgcacattcat | 1029 | NM_000077/ | SEQ ID |
| | inhibitor 2A (melanoma, | | | gtgggcatttcttgcgagcctcgcagcctccggaagctgtcgacttcatgacaag | | NM_058195/ | NO: 70 |
| | p16, inhibits CDK4)/ | | | cattttgtgaactagggaagctcaggggggttactggcttctcttgagtcacactgc | | NM_058197 | |
| | cyclin-dependent kinase | | | tagcaaatggca | | | |
| | inhibitor 2A (melanoma, | | | | | | |
| | p16, inhibits_CDK4)” | | | | | | |
| CDKN2A | “cyclin-dependent kinase | 209644_x_at | + | tgaggagccagcgtctagggcagcagccgcttcctagaagaccaggtcatgat | 1029 | NM_000077/ | SEQ ID |
| | inhibitor 2A (melanoma, | | | gatgggcagcgcccgagtggcggagctgctgctgctccacggcgcggagccc | | NM_058195/ | NO: 71 |
| | p16, inhibits CDK4)” | | | aactgcgccgaccgccactctcacctgacccgtgcacgacgctgcccggg | | NM_058197 | |
| | | | | agggcttcctggacacgctggtggtgctgcaccgggccggggcgcggctggac | | | |
| | | | | gtgcgcgatgcctggggccgtctgcccgtggacctggctgaggagctgggccat | | | |
| | | | | cgcgatgtcgcacggtacctgcgcgcggctgcggggggcaccagaggcagta | | | |
| | | | | accatgcccgcatagatgccacggaaggtccctcagacatccccgattgaaag | | | |
| | | | | aaccagagaggctctgagaaacctcgggaaacttagatcatcagtcaccgaa | | | |
| | | | | ggtcctacagggccacaactgcccccgccacaacccaccccgctttcgtagtttt | | | |
| | | | | catttagaaaatagagcttttaaaaatgtcctgccttttaacgtagatatatgccttcc | | | |
| | | | | ccc | | | |
| CEACAM | carcinoembryonic | 206199_at | + | atattagttaccctgggcatctaaaacctttaaatgtttgcatgcagccat | 1087 | NM_006890 | SEQ ID |
| 7 | antigen-related cell | | | tcgtcaaatgtcaaatattctctctttggctggaatgacaaaaactcaaataaatgt | | | NO: 72 |
| | adhesion molecule 7 | | | atgattaggaggacatcataacctatgaatgatggaagtccaaaatgatggtaa | | | |
| | | | | ctgacagtagtgttaatgccttatgtttagtcaaactctcatttaggtgacagcctggt | | | |
| | | | | gactccagaatggagccagtcatgctaaatgccatatactcacactgaaacatg | | | |
| | | | | aggaagcaggtagatcccagaacagacaaaactitcctaaaaacatgagagt | | | |
| | | | | ccaggcgtctgagtcagcacagtaagaaagtcctttctgctttaactcttagaaa | | | |
| | | | | aaagtaatatgaagtattctgaaattaaccaatcagtttatttaaatcaatttatttat | | | |
| | | | | attcttctgttcctggattcccattttacaaaacccactgttctactgttgtattgcccag | | | |
| | | | | t | | | |
| CHORDC | “cysteine and histidine- | 218566_s_at | | ggatttgtgttcttacagtacttgaaaatatttaaggaagagatgaagctctgcagtt | 26973 | NM_012124 | SEQ ID |
| 1 | rich domain (CHORD)- | | | ttttctatgtgggatgattacttttttaaggaggattaattctgaggtagtatagtaact | | | NO: 73 |
| | containing, zinc binding | | | aaaggggaatatatgaattgtttaacaaattagaatttgtttacaactacttgaatttt | | | |
| | protein 1/cysteine and | | | taaattatgtcaaaacttacattacttgccaagcagtatgatgttataggaaacata | | | |
| | histidine-rich domain | | | aataagattacagaggtatcaatttggttaaaattcaccattttataagactaagca | | | |
| | (CHORD)-containing, zinc | | | ataatcttaacaacctctttcctgaatatttaaatgtgtttgtatggtgttatgactaatt | | | |
| | binding protein 1” | | | gttactgatttagagactaagccctcttaaaacctttagttaaatataaaaagaaat | | | |
| | | | | tatatatatcttgcctccctgatggaaaactatataaaattgtagacttaaaaggttt | | | |
| | | | | gtggaaatacattaggatatcagaaaactaaatatatggagttgctttatgactatt | | | |
| CLU | “clusterin (complement | 208791_at | + | ggctgcctgcggatgaaggaccatgtgacaagtgccgggagatcttgtctgtg | 1191 | NM_001831/ | SEQ ID |
| | lysis inhibitor, SP-40, | | | gactgttccaccaacaacccctcccaggctaagctgcggcgggagctcgacg | | NM_203339 | NO: 74 |
| | 40, sulfated glycoprotein | | | aatccctccaggtcgctgagaggttgaccaggaaatataacgagctgctaaagt | | | |
| | 2, testosterone-repressed | | | cctaccagtggaagatgctcaacacctcctccttgctggagcagctgaacgagc | | | |
| | prostate message 2, | | | agtttaactgggtgtcccggctggcaaacctcacgcaaggcgaagaccagtac | | | |
| | apolipoprotein J)” | | | tatctgcgggtcaccacggtggcttcccacactlctgactcggacgttccttccggt | | | |
| | | | | gtcactgaggtggtcgtgaagctctttgactctgatcccatcactgtgacggtccct | | | |
| | | | | gtagaagtctccaggaagaacccta | | | |
| CLU | “clusterin (complement | 208792_s_at | + | agcagctgaacgagcagtttaactgggtgtcccggctggcaaacctcacgcaa | 1191 | NM_001831/ | SEQ ID |
| | lysis inhibitor, SP-40, | | | ggcgaagaccagtactatctgcgggtcaccacggtggcttcccacacttctgact | | NM_203339 | NO: 75 |
| | 40, sulfated glycoprotein | | | cggacgttccccggtgtcactgaggtggtcgtgaagctctttgactctgatcccat | | | |
| | 2, testosterone-repressed | | | cactgtgacggtccctgtagaagtctccaggaagaaccctaaatttatggagac | | | |
| | prostate message 2, | | | cgtggcggagaaagcgctgcaggaataccgcaaaaagcaccgggaggagt | | | |
| | apolipoprotein J)/ | | | gagatgtggatgttgc | | | |
| | clusterin (complement | | | | | | |
| | lysis inhibitor, SP-40, | | | | | | |
| | 40, sulfated glycoprotein | | | | | | |
| | 2, testosterone-repressed | | | | | | |
| | prostate message 2, | | | | | | |
| | apolipoprotein J)” | | | | | | |
| COL4A1 | “collagen, type IV, alpha | 211980_at | | gaaagactgtgctgtcctttaacataggtttttaaagactaggatattgaatgtgaa | 1282 | NM_001845 | SEQ ID |
| | 1” | | | acatccgttttcattgttcacttctaaaccaaaaattatgtgttgccaaaaccaaac | | | NO: 76 |
| | | | | ccaggttcatgaatatggtgtctattatagtgaaacatgtactttgagcttattgttttta | | | |
| | | | | ttctgtattaaatattttcagggttttaaacactaatcacaaactgaatgacttgactt | | | |
| | | | | caaaagcaacaaccttaaaggccgtcatttcattagtatlcctcattctgcatcctg | | | |
| | | | | gcttgaaaaacagctctgttgaatcacagtatcagtattttcacacgtaagcacatt | | | |
| | | | | cgggccatttccgtggtttctcatgagctgtgttcacagacctcagcagggcatcg | | | |
| | | | | catggaccgcaggagggcagattcggaccact | | | |
| COL4A1 | “collagen, type IV, alpha | 211981_at | | tcggctactcttttgtgatgcacaccagcgctggtgcagaaggctctggccaagc | 1282 | NM_001845 | SEQ ID |
| | 1” | | | cctggcgtcccccggctcctgcctggaggagtttagaagtgcgccattcatcgag | | | NO: 77 |
| | | | | tgtcacggccgtgggacctgcaattactacgcaaacgcttacagcttttggctcgc | | | |
| | | | | caccatagagaggagcgagatgttcaagaagcctacgccgtccaccttgaag | | | |
| | | | | gcaggggagctgcgcacgcacgtcagccgctgccaagtctgtatgagaagaa | | | |
| | | | | cataatgaagcctgaccagctaatgtcacaacatggtgctacttcttcttctttttgtt | | | |
| | | | | aacagcaacgaaccctagaaatatatcctgtgtacctcactgtccaatatgaaa | | | |
| | | | | accgtaaagtgccttataggaatttgcgtaactaacacaccctgc | | | |
| CTSB | cathepsin B | 200838_at | | tccccctgtagactagtgccgtgggagtacctgctgcccagctgctgtggccccct | 1508 | NM_001908/ | SEQ ID |
| | | | | ccgtgatccatccatctccagggagcaagacagagacgcaggatggaaagc | | NM_147780/ | NO: 78 |
| | | | | ggagttcctaacaggatgaaagttcccccatcagncccccagtacctccaagc | | NM_147781/ | |
| | | | | aagtagctttccacatttgtcacagaaatcagag | | NM_147782/ | |
| | | | | | | NM_147783 | |
| CTSB | cathepsin B/cathepsin B | 200839_s_at | | tggtgttgggagccctttggagaacgccagtctccaggtccccctgcatctatcga | 1508 | NM_001908/ | SEQ ID |
| | | | | gtttgcaatgcacaacctctctgatcttgtgctcagcatgattctttaatagaagtttt | | NM_147780/ | NO: 79 |
| | | | | atttttcgtgcactctgctaatcatgtgggtgagccagtggaacagcgggagcctg | | NM_147781/ | |
| | | | | tgctggtttgcagattgcctcctaatgacgcggctcaaaaggaaaccaagtggtc | | NM_147782/ | |
| | | | | aggagttgtttctgacccactgatctctactaccacaaggaaaatagtttaggaga | | NM_147783 | |
| | | | | aaccagcttttactgttt | | | |
| DAB2 | “disabled homolog 2, | 201278_at | | ggaaacgttcccagttcattttcagtcctgttgtgagcacagttctgaagggtttatta | 1601 | NM_001343 | SEQ ID |
| | mitogen-responsive | | | ttgtcaaaataagttttgttttgttttgtttatgttgggtttttaatgttgtctcttgacccttaa | | | NO: 80 |
| | phosphoprotein | | | tgctcaggttcttgtgggagttaatcagccacatccaangttaccttgagggggaa | | | |
| | (Drosophila)” | | | gaagagggtgatgctcagaagctaaacaagacaggggccacatgaccctcta | | | |
| | | | | ttgattagcccaagtagaaagtcctgtggttttatgtttaatggtaatagttgatcat | | | |
| | | | | atatggcataattttctatcagcttcctactcagtcactataaacacagacttgaaat | | | |
| | | | | agtactttaaatgtccaaatacctaaatgtgctaaactggaggtaactatttctagg | | | |
| | | | | tagtgaatttttgaaagtcatgatcagccacacaactgttttgtacatact | | | |
| DAB2 | “disabled homolog 2, | 201280_s_at | | aatccttattgttcagagttgtttgggggttctgtttcagagcataaaacctaaaggtt | 1601 | NM_001343 | SEQ ID |
| | mitogen-responsive | | | atagtagaacaaggcaccttcttaaaagaaatcttgcttcagaccatcagttaca | | | NO: 81 |
| | phosphoprotein | | | gagaatttcctaaagtaaaattgaagcaactacaacttctccttagacactttgga | | | |
| | (Drosophila)” | | | atctaaccacttaaggacctttttaaagagatagcttctcttctttctgaagatcaattt | | | |
| | | | | ctccaaggccaagattgtccttttctcccatttcttgctagctattgcaaatgaggg | | | |
| | | | | aagaacattattcatctctcctcccctttttctgattcttttttcagtcagttttgctcctg | | | |
| | | | | ggttcaagtagtattaccaccctttcacaagcaacagactc | | | |
| DIAPH2 | diaphanous homolog 2 | 205726_at | + | gctcactacactattcattgcacacaaatgaatttttcactttttaagatgcattcttgg | 1730 | NM_006729/ | SEQ ID |
| | (Drosophila)/diaphanous | | | tgctcaaaccagatcgaagtttgtctctnaaagctattgtctgcacaggctgctgc | | NM_007309 | NO: 82 |
| | homolog 2 (Drosophila) | | | atgctctgttgttaaatggatggacaggctattctaaattttggttgatacttttgctact | | | |
| | | | | atgggcaattaacttgaaaaaaataatcgatcccaactctgtgctctgatgtacct | | | |
| | | | | cttctgccccttttatgacacctttgaccaaatgccttctatggttcacagtgcaggc | | | |
| | | | | acaaaactactgatacagaaggttctttacaagcttattttacataccgtgaat | | | |
| | | | | ccctcacctaaagggagaggtgaaagcaaagactgctttgaatgggtattgag | | | |
| | | | | ggagattgtgtccataccaagccaccctgaagaagtatttcacttgcagtagaac | | | |
| | | | | tgtggatttgtctgtcatttcaccttggaataaacacctatctctaagcaggacca | | | |
| | | | | a | | | |
| EMP1 | epithelial membrane | 201324_at | | caccaaattacctaggctgaggttagagagattggccagcaaaaactgtggga | 2012 | NM_001423 | SEQ ID |
| | protein 1/epitheilal | | | agatgaactttgtcattatgatttcattatcacatgattatagaaggctgtcttagtgc | | | NO: 83 |
| | membrane protein 1 | | | aaaaaacatacttacatttcagacatatccaaagggaatactcacattttgttaag | | | |
| | | | | aaagttgaactatgactggagtaaaccatgtattcccttatcttttactttttttctgtgac | | | |
| | | | | atttatgtctcatgtaatttgcattactctggtggattgttctagtactgtattgggcttctt | | | |
| | | | | cgttaat | | | |
| EMP1 | epithelial membrane | 201325_s_at | | ttatcgccctgagaagatctaccccagggagaatctgagacatcttgcctacttttc | 2012 | NM_001423 | SEQ ID |
| | protein 1 | | | tttattagctttctcctcatccatttcttttatacctttcctttttggggagttgttatgccatg | | | NO: 84 |
| | | | | atttttggtatttatgtaaaaggattattactaattctatttctctatgtttanctagttaag | | | |
| | | | | gaaatgttgagggcaagccaccaaattacctag | | | |
| EMP1 | epithetial membrane | 213895_at | | aaggggtatctttctgtgagcaataaggactggataaagactgcatatccttg | 2012 | NM_001423 | SEQ ID |
| | protein 1 | | | tgtcnnnnncagcancnatacaataaggagggttttaatgtgaagcaggcaat | | | NO: 85 |
| | | | | ctnccagccccttctggtcttggatgaaatagttgcacagagtattgcaccaana | | | |
| | | | | atacacaatggaggctgaaaagttcaacatattttaagtcaattaatcaaattgca | | | |
| | | | | ttgattcttgatgctttcttagaggcctacatgatttcttagattgctctgataaactatc | | | |
| | | | | ataaggggtccacntcccctcatttagctcccccagggatttcttttcccccatgtca | | | |
| | | | | tacacccagtcctaaatcaacccccaaggctatccttccatcccttctgcagagg | | | |
| | | | | gaacttttgtcagactctgcaacaaactcctagctctatccagagtgtcctctgctg | | | |
| | | | | ctaagattggtatctttctcctcaaaagcctggatggtgaatgggggtgcattagtc | | | |
| | | | | agaattctcc | | | |
| EREG | epiregulin/epiregulin | 205767_at | + | taaaaacctgtatctgacccactttgtaatttttgctccaatatccattctgtagactttt | 2069 | NM_001432 | SEQ ID |
| | | | | gaaaaaaaagtttttaatttgatgcccaatatattctgaccgttaaaaaattcttgttc | | | NO: 86 |
| | | | | atatgggagaagggggagtaatgacttgtacaaacagtatttctggtgtatatttta | | | |
| | | | | atgtttttaaaaagagtaatttcatttaaatatctgttattcaaatttgatgatgttaaat | | | |
| | | | | gtaatataatgtattttctttttattttgcactctgtaattgcactttttaagtttgaagagc | | | |
| | | | | cattttggaaacggtttttattaaagatgctatggaacataaagttgtattgcatgca | | | |
| | | | | atttaaagtaacttatttgactatgaatattatcggattactgaattgtatcaatttgttt | | | |
| | | | | gtgttcaatatcagctttgataattgtgtaccttaag | | | |
| FGF2 | fibroblast growth factor 2 | 204421_s_at | + | gggatcctatttagccttagtaccactaatcaaaagttcggcatgtagctcatgat | 2247 | NM_002006 | SEQ ID |
| | (basic) | | | ctatgctgtttctatgtcgtggaagcaccggatgggggtagtgagcaaatctgccc | | | NO: 87 |
| | | | | tgctcagcagtcaccatagcagctgactgaaaatcagcactgcctgagtagtttt | | | |
| | | | | gatcagtttaacttgaatcactaactgactgaaaattgaatgggcaaataagtgct | | | |
| | | | | tttgtctccagagtatgcgggagacccttccacctcaagatggatatttcttcccca | | | |
| | | | | aggatttcaagatgaattgaaatttttaatcaagatagtgtgctttattctgttg | | | |
| FGF2 | fibroblast growth factor 2 | 204422_s_at | + | atatcttcttcaggctctgacaggcctcctggaaacttccacatatttttcaactgca | 2247 | NM_002006 | SEQ ID |
| | (basic)/fibroblast growth | | | gtataaagtcagaaaataaagttaacataactttcactaacacacacatatgtag | | | NO: 88 |
| | factor 2 (basic) | | | atttcacaaaatccacctataattggtcaaagtggttgagaatatattttttagtaatt | | | |
| | | | | gcatgcaaaatttttctagcttccatcctttctccctcgtttcttctttttttgggggagctg | | | |
| | | | | gtaactgatgaaatcttttcccaccttttctcttcaggaaatataagtggttttgtttggt | | | |
| | | | | taacgtgatacattctgtatgaatgaaacattggagggaaacatctactgaatttct | | | |
| | | | | gtaatttaaaatattttgctgctagttaactatgaacagatagaagaatcttacagat | | | |
| | | | | gctgctataaataagtagaaaatataaatttcatcactaaaatatgctattttaaaat | | | |
| | | | | ctatttcctatattgtatttctaatcagatgtattactcttattatttctat | | | |
| FLJ22662 | hypothetical protein | 218454_at | + | gtggctatccacttagttcagaagctgggcttggacactcttatgatttagctcc | 79887 | NM_024829 | SEQ ID |
| | FLJ22662 | | | acgagccaaaattttccggcgtgaccaagggaaagtgactgatacggcatcca | | | NO: 89 |
| | | | | tgaaatatatcatgcgatacaacaattataagaaggatccttacagtagaggtga | | | |
| | | | | cccctgtaataccatctgctgccgtgaggacctgaactcacctaacccaagtcct | | | |
| | | | | ggaggttgttatgacacaaaggtggcagatatctacctagcatctcagtacacat | | | |
| | | | | cctatgccataagtggtcccacagtacaaggtggcctccctgtttttcgctgggac | | | |
| | | | | cgtttcaacaaaactctacatcagggcatgccagaggtctacaactttgattttatt | | | |
| | | | | accatgaaaccaattttgaaacttgatataaaatgaaggagggagatgacgga | | | |
| | | | | ctagaagactgtaaataagataccaaaggcactattttagctatgtttttcccatca | | | |
| | | | | gaat | | | |
| GADD45 | “growth arrest and DNA- | 207574_s_at | | ccccatcacggagggtccagactgtccactcgggggtggagtgagactgactg | 4816 | NM_015675 | SEQ ID |
| B | damage-inducible, beta/ | | | caagccccaccctccttgagactggagctgagcgtctgcatacgagagacttgg | | | NO: 90 |
| | growth arrest and DNA- | | | ttgaaacttggttggtccttgtctgcaccctcgacaagaccacactttgggacttgg | | | |
| | damage-inducible, beta” | | | gagctggggctgaagttgctctgtacccatgaactcccagtttgcgaattaataag | | | |
| | | | | agacaatctattttgttacttgcacttgttattcgaaccactgagagcgagatgg | | | |
| HIF1A | “hypoxia-inducible factor | 200989_at | | tcatctgatgtttctatagtcactttgccagctcaaaagaaaacaataccctatgta | 3091 | NM_001530/ | SEQ ID |
| | 1, alpha subunit (basic | | | gttgtggaagtttatgctaatattgtgtaactgatattaaacctaaatgttctgcctac | | NM_181054 | NO: 91 |
| | helix-loop-helix | | | cctgttggtataaagatattttgagcagactgtaaacaagaaaaaaaaaatcatg | | | |
| | transcription factor)/ | | | cattcttagcaaaattgcctagtatgttaatttgctcaaaatacaatgtttgattttatg | | | |
| | hypoxia-inducible factor | | | cactttgtcgctattaacatcctttttttcatgtagatttcaataattgagtaattttagaa | | | |
| | 1, alpha subunit (basic | | | gcattattttaggaatatatagttgtcacagtaaatatcttgttttttctatgtacattgta | | | |
| | helix-loop-helix | | | caaatttttcattccttttgctctttgtggttggatctaacactaactgt | | | |
| | transcription factor)” | | | | | | |
| HOP | homeodomain-only | 211597_s_at | + | aagctatgtgtatcttctgtgtaaagcagtggcttcactggaaaaatggtgtggcta | 84525 | NM_932495/ | SEQ ID |
| | protein/homeodomain- | | | gcatttccctttgagtcatgatgacagatggtgtgaaaaccatctaagtttgcttttg | | NM_139211/ | NO: 92 |
| | only protein | | | accatcacctcccagtagcaatttgctttcataatccatttagcaatccaggcctct | | NM_139212 | |
| | | | | gttgaaaagataatatgagggagaagggaacacatttccttctgaacttacttcc | | | |
| | | | | ctaagtcactttccttatgtatcatctaatacaatgatggttgagtgaaaatacaga | | | |
| | | | | aggggtgtttgagattcagatttcataaaacacttccttggaatatagctgcattaa | | | |
| | | | | cttggaaagaagcctgttgggccagaagacacga | | | |
| HOXC10 | homeo box C10/homeo | 218959_at | | gctggtgtgtgtgtcaaaccctcactcacccacgcactcacacacagcattctgtt | 3226 | NM_017409 | SEQ ID |
| | box C10 | | | ctccatgcaaagttaagatcgaatccatccgcttgtaggggaaaaaaaggaaa | | | NO: 93 |
| | | | | aaaattaaccagagagggtctgtaatctcgcagagcacaggcagaatcgttcct | | | |
| | | | | tccttgctgcatttcctccttagactaatagacgttttggaaagttcggctagtgttcgt | | | |
| | | | | gtgtttgtcgtagcacccagagcctccaccaaaccctctccatgtctttacctccca | | | |
| | | | | gtcgctctaagatctgcttgaagtctcgtatttgtactgctttctgcttttctcccacccc | | | |
| | | | | tcctagcacccccacatcccccatctagtaacatctcagaaatttcatccagagg | | | |
| | | | | aacaaaaaaattaaaaatagaacatagcaaagcaaagacagaatgcccccc | | | |
| | | | | cccaaatattgtcctgtccctgtctgggagttgtgttatttaaagatattctgtatgttgt | | | |
| | | | | atcttttgcatgtagcttccttaat | | | |
| ITGB2 | “integrin, beta 2 (antigen | 202803_s_at | | atctggaaggctctgatccacctgagcgacctccgggagtacaggcgctttgag | 3689 | NM_000211 | SEQ ID |
| | CD18 (p95), lymphocyte | | | aaggagaagctcaagtcccagtggaacaatgataatccccttttcaagagcgc | | | NO: 94 |
| | function-associated | | | caccacgacggtcatgaaccccaagtttgctgagagttaggagcacttggtgaa | | | |
| | antigen 1; macrophage | | | gacaaggccgtcaggacccaccatgtctgccccatcacgcggccgagacatg | | | |
| | antigen 1 (mac-1) beta | | | gcttggccacagctcttgaggatgtcaccaattaaccagaaatccagttattttcc | | | |
| | subunit)/integrin, beta 2 | | | gccctcaaaatgacagccatggccggccggtgcttctgggggctcgtcggggg | | | |
| | (antigen CD18 (p95), | | | gacagctccactctgactggcacagtctttgcatggagacttgaggagggcttga | | | |
| | lymphocyte function- | | | ggttggtgaggttaggtgcgtgtttcctgtgcaagt | | | |
| | associated antigen 1; | | | | | | |
| | macrophage antigen 1 | | | | | | |
| | (mac-1) beta subunit)” | | | | | | |
| KHDRBS | “KH domain containing, | 209781_s_at | | cagcccggccagttggagttgtagtaccacgagggacgccaactcccagagg | 10656 | NM_006558 | SEQ ID |
| 3 | RNA binding, signal | | | agtcctgtccacccgagggccagtgagtcggggaagaggacttctcactccca | | | NO: 95 |
| | transduction associated 3/ | | | gagcaagaggagtccccccaactgggtacagacctccaccgccacccccga | | | |
| | KH domain containing, | | | cacaagagacttatggagaatatgactatgatgatggatatggcactgcttatgat | | | |
| | RNA binding, signal | | | gaacagagttatgattcctatgataacagctatagcaccccagcccaaagtggt | | | |
| | transduction associated | | | gctgattactatgattacggacatggactcagtgaggagacttatgattcctacgg | | | |
| | 3” | | | gcaagaagagtggactaactcaagacacaaggcaccttcagcgaggacagc | | | |
| | | | | aaagggcgtctacagagaccagccatatggcagatactgattgtactgtctgatg | | | |
| | | | | ttgtgaaatagccaatctccaccagtcctgtatactg | | | |
| KRT13 | keratin 13/keratin 13 | 207935_s_at | + | gagaacacggtggcagagacggagtgccgctatgccctgcagctgcagcag | 3860 | NM_002274/ | SEQ ID |
| | | | | atccagggactcatcagcagcatcgaggcccagctgagcgagctccgcagtg | | NM_153490 | NO: 96 |
| | | | | agatggagtgccagaaccaagagtacaagatgctgctggacatcaagacacg | | | |
| | | | | tctggagcaggagatcgccacctaccgcagcctgctcgagggccaggacgcc | | | |
| | | | | aagaagcgtcagcccccgtagcacctctgttaccacgacttctagtgcctctgtta | | | |
| | | | | ccaccacctctaatgcctctggtcgccgcacttctgatgtccgtaggccttaaatct | | | |
| | | | | gcctggcgtcccctccctctgtcttcagcacccagaggaggagagagccggca | | | |
| | | | | gttccctgcaggagagaggaggggctgctggacccaaggctcagtccctctgct | | | |
| | | | | ctcaggaccccctgtcctgactctctcctgatggtgggccctctgtgctcttctcttcc | | | |
| | | | | ggtcggatctctctcctctctgacctggatacgctttggtttctcaacttc | | | |
| LASS6 | LAG1 longevity assurance | 212446_s_at | + | aactttaacttagagcttcattactttaagaatggaaaacaacctctgagtttgattt | 25378 | NM_203463 | SEQ ID |
| | homolog 6 (S. cerevisiae) | | | cccaaagtttcataaagcccctaagctcatgattttcatcaactctttgcccacata | 2 | | NO: 97 |
| | | | | gtcatttacctccacagccgtttgttgtcatagaaggggtggtggtgtttggatttgat | | | |
| | | | | ttttttcaacttgcagtgagaaataggataggtgacaaaaccttacttgttttcttaag | | | |
| | | | | acaattcagtgcttgagcatctctgtcagaaatggaatgaaatactgttagccaat | | | |
| | | | | tagaattattttatgtattgttattgtgttttgctgatttttatatgaaaatataattattcatt | | | |
| | | | | cttgatctctggaagcaa | | | |
| LTBP2 | latent transforming growth | 204682_at | | gggagccaaggctttatacgtctaaagaaaatattcagtagctgaatccgccca | 4053 | NM_000428/ | SEQ ID |
| | factor beta binding pro- | | | gtgatagcctgtgggcaccagcagcaagggctgccatgggatacagcaccca | | NM_032035 | NO: 98 |
| | tein 2/latent transforming | | | tctacaaagacctctattacataaacactgcttcttacaggaaacaaacctcttctg | | | |
| | growth factor beta binding | | | ggatctccttttgtgaaaaccagtttgatgtgctaaaagtaaaaagtctattttccag | | | |
| | protein 2 | | | tgtggtcttgttcagaagcagccagatttccaatgttgtttttcccctccactcagaa | | | |
| | | | | acccctgccctttcccttcagaaaacgatggcaggcattcctctgagtttacaagc | | | |
| | | | | agagactcactccaacccaaactagctggg | | | |
| MAP4K5 | mitogen-activated protein | 203552_at | | acacacacatgcaattttgcttaacaaaagtattttataatacagtttcatacagaa | 11183 | NM_006575/ | SEQ ID |
| | kinase kinase kinase | | | ttaccttaaaagggagtcttatgttttcaactacagatagttgtaagggatcntaca | | NM_198794 | NO: 99 |
| | kinase 5 | | | gaagatattgatgatagttgaaatattcttagaaggggtgtgtatgtctagctgtgtc | | | |
| | | | | taccatgtgtatgtattcttgacaagcagtataaaatacctgtgatttttctttacatta | | | |
| | | | | gggataatgcataaggaattaatcttcatatatattatcatccctaatgtagcaggg | | | |
| | | | | ggaagtatttaattgcccatgatatgtattttacttatactatgccagagaggaaact | | | |
| | | | | ataaagtaattacacatgtaatcttgggtttttcacatatgtaggtattcattttgagta | | | |
| | | | | ggttgaagaagaaaaaaaatatttaaatgaattgaattcctgatgggatagtatc | | | |
| | | | | aat | | | |
| MAP4K5 | mitogen-activated protein | 203553_s_at | | gaactctgcatcttcatggtttacagaaattggtgcaggcagccagcagttagatt | 11183 | NM_006575/ | SEQ ID |
| | kinase kinase kinase | | | ccattcatgtaacacagttggagagagataccgttttagtgtgtttagacaaatttgt | | NM_198794 | NO: 100 |
| | kinase 5 | | | gaaaattgtaaatctacaaggaaaattaaaatcaagtaagaaactggcctctga | | | |
| | | | | gttaagttttgattttcgcattgaatctgtagtatgccttcaagacagtgtgttggctttc | | | |
| | | | | tggaaacatgggatgcagggtaaaagcttcaagtcagatgaggttacccagga | | | |
| | | | | gatttcagatgaaacaagagttttccgcttattaggatcagacagggttgtcgttttg | | | |
| | | | | gaaagtaggccaacagaaaatcctactgcacacagcaatctctacatcttggct | | | |
| | | | | ggaatgaaaatagttactaagcaacagaaactgatctcaaatgacaggaaa | | | |
| | | | | atgaatatactccattgaaagggaaaataaggaaattcaatacaaactgcacta | | | |
| | | | | tgatttgctttaact | | | |
| MMP2 | “matrix metalloproteinase | 201069_at | | ctcagagccacccctaaagagatcctttgatattttcaacgcagccctgctttggg | 4313 | NM_004530 | SEQ ID |
| | 2 (gelatinase A, 72 kDa | | | ctgccctggtgctgccacacttcaggctcttctcctttcacaaccttctgtggctcac | | | NO: 101 |
| | gelatinase, 72kDa type IV | | | agaacccttggagccaatggagactgtctcaagagggcactggtggcccgac | | | |
| | collagenase)/matrix | | | agcctggcacagggcagtgggacagggcatggccaggtggccactccagac | | | |
| | metalloproteinase 2 | | | ccctggcttttcactgctggctgccttagaacctttcttacattagcagtttgctttgtat | | | |
| | (gelatinase A, 72 kDa | | | gcactttgttttttctttgggtcttgttttttttttccacttagaaattgcatttcctgacaga | | | |
| | gelatinase, 72 kDa type IV | | | aggactcaggttgtctgaagtcactgcacagtgcatctcagcccacatagtgatg | | | |
| | collagenase)” | | | gttcccctgttcactctacttagcatgtccctaccgagtctcttctccactggatgga | | | |
| | | | | ggaaaaccaagccgtggcttcccgctcagccctccctgcccctcccttcaaccat | | | |
| | | | | tccccatgggaaat | | | |
| MYC | v-myc myelocytomatosis | 202431_s_at | + | gcaacaaccgaaaatgcaccagccccaggtcctcggacaccgaggagaatg | 4609 | NM_002467 | SEQ ID |
| | viral oncogene homolog | | | tcaagaggcgaacacacaacgtcttggagcgccagaggaggaacgagctaa | | | NO: 102 |
| | (avian)/(v-myc | | | aacggagcttttttgccctgcgtgaccagatcccggagttggaaaacaatgaaa | | | |
| | myelocytomatosis viral | | | aggcccccaaggtagttatccttaaaaaagccacagcatacatcctgtccgtcc | | | |
| | oncogene homolog | | | aagcagaggagcaaaagctcatttctgaagaggacttgttgcggaaacgacg | | | |
| | (avian) | | | agaacagttgaaacacaaacttgaacagctacggaactcttgtgcgtaaggaa | | | |
| | | | | aagtaaggaaaacgattccttctaacagaaatgtcctgagcaatcacctatgaa | | | |
| | | | | cttgtttcaaatgcatgatcaaatgcaacctcacaaccttggctgagtc | | | |
| NRP1 | neuropilin 1 | 210510_s_at | | aacatccgcctggtaaccagtcgctctggctgggcacttccacccgcacctcatt | 8829 | NM_003873 | SEQ ID |
| | | | | cctacatcaatgagtggctccaaatagacctgggggaggagaagatcgtgag | | | NO: 103 |
| | | | | gggcatcatcattcagggtgggaagcaccgagagaacaaggtgttcatgagg | | | |
| | | | | aagttcaagatcgggtacagcaacaacggctcggactggaagatgatcatgg | | | |
| | | | | atgacagcaaacgcaaggcgaagtcttttgagggcaacaacaactatgataca | | | |
| | | | | cctgagctgcggacttttccagctctctccacgcgattcatcaggatctaccccga | | | |
| | | | | gagagccactcatggcggactggggctcagaatggagctgctgggctgtgaag | | | |
| | | | | tggaagcccctacagctggaccgaccactcccaacgggaacttggtggatgaa | | | |
| | | | | tgtgatgacgaccaggccaactgccacagtggaaca | | | |
| NRP1 | neuropilin 1 | 212298_at | | gcaaatatcttaccaggcagcctatgaattaacccaaagaagctttggttggttttg | 8829 | NM_003873 | SEQ ID |
| | | | | gtggatttttatcatgccatgttggacatgagattttttagatcttccttcccacattgct | | | NO: 104 |
| | | | | agacgtctcactcaaagacatttgttgggagtcacatttgcatcatagangagac | | | |
| | | | | agtccattcatcttagttaaattggattgagaatgccttttgtttccaggaaaatattg | | | |
| | | | | atcaccatgaaagaagaatagttttttgtccccagagacattcatttagttgatata | | | |
| | | | | atcctaccagaaggaaagcactaagaaacactcgtttgttgtttttaaaggcaac | | | |
| | | | | agacttaaagttgtcctcagccaagg | | | |
| OLFM1 | olfactomedin 1/ | 205591_at | + | caggtgtatctgcacagtggtcgccccacagcagaccatgtgttcacgggatgc | 10439 | NM_006334/ | SEQ ID |
| | olfactomedin 1 | | | ccgcacaaaacagctgaggcagctactggagaaggtgcagaacatgtctcaa | | NM_014279/ | NO: 105 |
| | | | | tccatagaggtcttggacaggcggacccagagagacttgcagtacgtggagaa | | NM_058199 | |
| | | | | gatggagaaccaaatgaaaggactggagtccaagttcaaacaggtggagga | | | |
| | | | | gagtcataagcaacacctggccaggcagtttaagggctaacttaaaagagttttt | | | |
| | | | | tcaatgctgcagtgactgaagaagcagtccactcccatgtaaccatgaaagag | | | |
| | | | | agccagagagctttttgcaccatgcatttttactattattttccaatacttagcaccatt | | | |
| | | | | tcactaaggaaccttgaatacaaccaggatcctcctttgcatgcgactgtagctgc | | | |
| OLFM1 | olfactomedin 1 | 213131_at | + | gcgggccacagacgtcggaagaaactcccgtatttgcagctggaactgcagcc | 10439 | NM_006334/ | SEQ ID |
| | | | | cacggcgccccggttttcctccccgccctgtccctctctggtcaaacaacatacta | | NM_014279/ | NO: 106 |
| | | | | aagaggcgaggcaatgactgttggccagttctcaccggggaaaaacccnact | | NM_058199 | |
| | | | | gttaggatggcatgaacatttccttagatcgtggtcagctccgaggaatgtggcn | | | |
| | | | | nccaggctctttgangagccatgggctgcacccnggccgtaggcntagtgtaa | | | |
| | | | | ctcgcatcccattgcagtgccngrncnttgactgtgttgctgtctcttagattaaccgt | | | |
| | | | | gctgaggctccnacatagctccntggacctgtgtcntagtacatactgaagcgat | | | |
| | | | | ggtcagagtgtgtagagtgaagttgctgtgcccacattgtttgaactcgcgtaccc | | | |
| | | | | cgtagatacattgtgcaacgttcttctgttattcccttgaggtggtaacttcgtatgttc | | | |
| | | | | agt | | | |
| OSMR | oncostatin M receptor/ | 205729_at | + | ggagacttgagcttgacctaaggatatgcattaaccactctacagactcccactc | 9180 | NM_003999 | SEQ ID |
| | oncostatin M receptor | | | agtactgtacagggtggctgtggtcctagaagttcagtttttactgaggaaatatttc | | | NO: 107 |
| | | | | cattaacagcaattattatattgaaggctttaataaaggccacaggagacattact | | | |
| | | | | atagcatagattgcaaatgaaatttactgagcgtgttttataaaaaactcacagg | | | |
| | | | | tgtttgaggccaaaacagattttagacttaccttgaacggataagaatctatagttc | | | |
| | | | | actgacacagtaaaattaactctgtgggtgggggcggggggcatagctctaatc | | | |
| | | | | taatatataaaatgtgtgatgaatcaacaagatttccacaattcttctgtcaagctta | | | |
| | | | | ctacagtgaaagaatgggattggcaagtaacttctgacttactgtcagttgtacttc | | | |
| | | | | tgctccatagacatcagtattctgccatcatttttgatgactacctcagaacataaa | | | |
| | | | | aaggaacgtatatcacataattccagtcacagtttttggttcctctt | | | |
| PCDHAC | “protocadherin alpha | 210674_s_at | | ctgacctctttgaagttgcagaatgctttgaaattctaatggtatctgaaatatcagc | 56134 | NM_018899/ | SEQ ID |
| 2 | subfamily C, 2/ | | | tcatagaaagtaacaaaatttgctgtcaccttaaataagacattttaattttgttataa | | NM_031883 | NO: 108 |
| | protocadherin alpha | | | tgtacaatttagaagtttgattaattatattatctatttaggcattaatataaaagaggt | | | |
| | subfamily C, 2” | | | aggagtctgttatttaaaaaaagcattaaatttaaaaaaaaactgtcttgtctactttt | | | |
| | | | | agcttcattctcccatattttgaagggtgtgtaacttcagctctgcaggattgcatgg | | | |
| | | | | ggtaaaacttgttaccaacacatgtgaaccattgctacattgtaggttgtgatcattt | | | |
| | | | | tgccccactgaagcccatgtatctgaccttacgtgccttttgaactaggagaatcg | | | |
| | | | | ggctaatttattaatgatgataattataatgtatctgtacagcactttttacatttgcga | | | |
| | | | | agtgctttccaatccatgttagttactagttattacagctgtaaggataaaacacgtc | | | |
| | | | | atgtggattcattttga | | | |
| PDGFRA | “platelet-derived growth | 203131_at | | agaaaatttgccaatctttcctactttctatttttatgatgacaatcaaagccggcctg | 5156 | NM_006206 | SEQ ID |
| | factor receptor, alpha | | | agaaacactatttgtgactttttaaacgattagtgatgtccttaaaatgtggtctgcc | | | NO: 109 |
| | polypeptide/platelet- | | | aatctgtacaaaatggtcctatttttgtgaagagggacataagataaaatgatgtt | | | |
| | derived growth factor | | | atacatcaatatgtatatatgatttctatatagacttggagaatactgccaaaacat | | | |
| | receptor, alpha | | | ttatgacaagctgtatcactgccttcgtttatatttttttaactgtgataatccccacag | | | |
| | polypeptide” | | | gcacattaactgttgcacttttgaatgtccaaaatttatattttagaaataataaaaa | | | |
| | | | | gaaagatacttacatgttcccaaacaatggtgtggtgaatgtgtgagaaaaact | | | |
| | | | | aacttgatagggtctaccaatacaaaatgtattacgaatgcccctgttcatgtt | | | |
| PDZK1 | PDZ domain containing 1/ | 205380_at | | gtcaaaccatgactcgcacatggcaaaagaacgggcccacagtacagcctca | 5174 | NM_002614 | SEQ ID |
| | PDZ domain containing | | | cattcttcttccaattctgaagatacagagatgtgatgaaaacaagtaatagctttg | | | NO: 110 |
| | 1 | | | gctgtttatttgatagctgtttctgggtatttaataggaatcctttctcaaggaatgagtt | | | |
| | | | | gtgacctgtttactgtctctttagaagaaaaactccactggaaaccattcaccatgt | | | |
| | | | | gtgaccttctttatcatttgtcttacaggcggctattgcagacggctaatttatgc | | | |
| | | | | ttaacttaggaagagataaggcaagagctagatttttttcatgtgatcttttccaagc | | | |
| | | | | ttcaacttaacttaactacatttctctgtatgatgatgtctcttacttctacaggttccttg | | | |
| | | | | agcac | | | |
| PEA15 | phosphoprotein enriched | 200788_s_at | | taaattcacatgcagtctcagagactatttagacaaagttcaagttaggagctttta | 8682 | NM_003768/ | SEQ ID |
| | in astrocytes 15/ | | | ggatgtgggagtaaaactttaatgggaggggagggctggctgctggaagaag | | NM_013287 | NO: 111 |
| | phosphoprotein enriched | | | gaagaagccagactggttagacagtactcttaactcctagcccagcctacgtgc | | | |
| | in astrocytes 15 | | | cctgcccctctggccactgctgcagacacctgccttaacacacacacctctagg | | | |
| | | | | actccacagttttgccttaaaggaccttcccaagtctccctttccctgtctggcttctc | | | |
| | | | | ccttaagaagagagagatacttgtagaattgggtggggggaatgagcatgaac | | | |
| | | | | tgccttccatttgggatatttacattagagtgagagagagaataaggagcctttc | | | |
| | | | | ttatggaagaaatgggagaagagagacagggttcttttcagcagagtctagtag | | | |
| | | | | tttcctgtaaggcaaaataatctaaaaagactaacctgcccacccactccttatat | | | |
| | | | | tgctgtgagattgcccc | | | |
| PGM1 | phosphoglucomutase 1/ | 201968_s_at | | cggacccatccaagtcatctgattgaagagcatgacagaaacaaaatgtattca | 5236 | NM_002633 | SEQ ID |
| | phosphoglucomutase 1 | | | ccaagcattttaggatttgactttttcactaaccagttgacgagcagtgcatttacaa | | | NO: 112 |
| | | | | ggcactgccaaacaagatgcccttgggagctgtgagggaaagaggacctgcg | | | |
| | | | | ggcttagatcaatctcaattccttttcatgccctcctgcattgctgctgcgtgggtattt | | | |
| | | | | gtctccttagccatcaggtacagtttacactacaatgtaagctataggtggagcat | | | |
| | | | | cagcagtgagtgaggccattcttcatccttaggatgtggcaatgaaatgatggtg | | | |
| | | | | caagttcctttctcttttgtgaatctttccccccatttcctgtttacatgtaacccaacaa | | | |
| | | | | aatgcaatttctagtgccttctgtccaatcagttctttcctctgagtgagacgtacttg | | | |
| | | | | gctacagatttctgccttgttttgcgacattgtc | | | |
| PI3 | “protease inhibitor 3, | 203691_at | | gattggtatggccttagctcttagccaaacaccttcctgacaccatgagggccag | 5266 | NM_002638 | SEQ ID |
| | skin- derived (SKALP)” | | | cagcttcttgatcgtggtggttcctcatcgctgggacgctggttctagaggcagc | | | NO: 113 |
| | | | | tgtcacgggagttcctgttaaaggtcaagacactgtcaaaggccgtgttccattca | | | |
| | | | | atggacaagatcccgttaaaggacaagtttcagttaaaggtcaagataaagtca | | | |
| | | | | aagcgcaagagccagtcaaaggtccagtctccactaagcctggctcctgcccc | | | |
| | | | | attatcttgatccggtgcgccatgttgaatccccctaaccgctgcttgaaagatact | | | |
| | | | | gactgcccaggaatcaagaagtgcgtgaaggctcttgcgggatggcctgtttcg | | | |
| | | | | ttccccagtgaagggagccgcttccttgct | | | |
| PLAU | “plasminogen activator, | 205479_s_at | | cccgaccggtgggcatttgtgaggcccatggttgagaaatgaataatttcccaatt | 5328 | NM_002658 | SEQ ID |
| | urokinase/plasminogen | | | aggaagtgtaagcagctgaggtctcttgagggagcttagccaatgtgggagca | | | NO: 114 |
| | activator, urokinase” | | | gcggtttggggagcagagacactaacgacttcagggcagggctctgatattcca | | | |
| | | | | tgaatgtatcaggaaatatatatgtgtgtgtatgtttgcacacttgttgtgtgggctgt | | | |
| | | | | gagtgtaagtgtgagtaagagctggtgtctgattgttaagtctaaatatttccttaaa | | | |
| | | | | ctgtgtggactgtgatgccacacagagtggtctttctggagaggttataggtcactc | | | |
| | | | | ctggggcctcttgggtcccccacgtgacagtgcctgggaatgtacttattctgcag | | | |
| | | | | catgacctgtgaccagcactgtctcagtttcactttcacatagatgcccttcttggc | | | |
| | | | | cagttatcccttccttttagcctagttcatccaatcctcactgggtgggg | | | |
| PLAU | “plasminogen activator, | 211668_s_at | | accacaacgacattgcctgctgaagatccgttccaaggagggcaggtgtgcg | 5328 | NM_002658 | SEQ ID |
| | urokinase/plasminogen | | | cagccatcccggactatacagaccatctgcctgccctcgatgtataacgatcccc | | | NO: 115 |
| | activator, urokinase” | | | agtttggcacaagctgtgagatcactggctttggaaaagagaattctaccgacta | | | |
| | | | | tctctatccggagcagctaagatgactgttgtgaagctgatttcccaccgggagt | | | |
| | | | | gtcagcagccccactactacggctctgaagtcaccaccaaaatgctgtgtgctg | | | |
| | | | | ctgacccacagtggaaaacagattcctgccagggagactcagggggacccct | | | |
| | | | | cgtctgttccctccaaggccgcatgactttgactggaattgtgagctggggccgtg | | | |
| | | | | gatgtgccctgaaggacaagccaggcgtctacacgagagtctcacactt | | | |
| PPIF | peptidylprolyl Isomerase F | 201489_at | | gggttgccatccaagtgaaagtcttttccttgaccaagggggacagtcagttttgc | 10105 | NM_005729 | SEQ ID |
| | (cyclophilin F)/ | | | aaaaggactctaatacctgtttaatattgtcttcctaattgggataatttaattaacaa | | | NO: 116 |
| | peptidylprolyl isomerase F | | | gattgactagaagtgaaactgcaacactaacttccccgtgctgtggtgtgacctg | | | |
| | (cyclophilin F) | | | agttggtgacacaggccacagaccccagagcttggcttttgaaacacaactca | | | |
| | | | | gggcttttgtgaaggttcccccgctgagatcmcctcctggttactgtgaagcctgtt | | | |
| | | | | ggtttgctgctgtcgtttttgaggagggcccatgggggtaggagcagttgaacctg | | | |
| | | | | ggaacaaacctcacttgagctgtgcctagacaatgtgaattcctgtgttgctaaca | | | |
| | | | | gaagggcctgtaagctcctgtgctccggagggaagcatttcctggtaggctttg | | | |
| PPIF | peptidylprolyl isomerase F | 201490_s_at | | gctgaaggcagatgtcgtcccaaagacagctgagaacttcagagccctgtgca | 10105 | NM_005729 | SEQ ID |
| | (cyclophilin F) | | | ctggtgagaagggcttcggctacaaaggctccaccttccacagggtgatcccttc | | | NO: 117 |
| | | | | cttcatgtgccaggcgggcgacttcaccaaccacaatggcacaggcgggaagt | | | |
| | | | | ccatctacggaagccgctttcctgacgagaactttacactgaagcacgtggggc | | | |
| | | | | caggtgtcctgtccatggctaatgctggtcctaacaccaacggctcccagttcttc | | | |
| | | | | atctgcaccataaagacagactggttggatggcaagcatgttgtgttcggtcacgt | | | |
| | | | | caaagagggcatggacgtcgtgaagaaaatagaatctttcggctctaagagtg | | | |
| | | | | ggaggacatccaagaagattgtcatcacagactgtggccagttgagctaatctgt | | | |
| | | | | ggccagggtgctggcatggtggcagctgcaaatgtccatgcacccaggtggcc | | | |
| | | | | gcgttgggctgtcagccaaggtgcctgaaacgatacgtgtgcccact | | | |
| PTGS2 | prostaglandin- | 204748_at | + | ggttgaatgtttgtccttaggataggcctatgtgctagcccacaaagaatattgtctc | 5743 | NM_000963 | SEQ ID |
| | endoperoxide synthase 2 | | | attagcctgaatgtgccataagactgaccttttaaaatgttttgagggatctgtggat | | | NO: 118 |
| | (prostaglandin G/H | | | gcttcgttaatttgttcagccacaatttattgagaaaatattctgtgtcaagcactgtg | | | |
| | synthase and | | | ggttttaatatttttaaatcaaacgctgattacagataatagtatttatataaataattg | | | |
| | cyclooxygenase)/ | | | aaaaaaattttcttttgggaagagggagaaaatgaaataaatatcattaaagata | | | |
| | prostaglandin- | | | actcaggagaatcttctttacaattttacgtttagaatgtttaaggttaagaaagaaa | | | |
| | endoperoxide synthase 2 | | | tagtcaatatgcttgtataaaacactgttcactgttttttttaaaaaaaaaacttgattt | | | |
| | (prostaglandin G/H | | | gttattaacattgatctgctgacaaaacctgggaatttgggttgtgtatgcgaatgttt | | | |
| | synthase and | | | cagtgcctcagacaaat | | | |
| | cyclooxygenase) | | | | | | |
| RRM1 | ribonucleotide reductase | 201476_s_at | + | gaacaagcgtcctggggcatttgctatttacctggagccttggcatttagacatcttt | 6240 | NM_001033 | SEQ ID |
| | M1 polypeptide | | | gaattccttgatttaaagaagaacacaggaaaggaagagcagcgtgccagag | | | NO: 119 |
| | | | | atcttttctttgctctttggattccggatctcttcatgaaacgagtggagactaatcag | | | |
| | | | | gactggtctttgatgtgtccaaatgagtgtcctggtctggatgaggtttggggagag | | | |
| | | | | gaatttgagaaactatatgcaagttatgagaaacaaggtcgtgtccgcaaagttg | | | |
| | | | | taaaagctcagcagctttggtatgccatcattgagtctcagacggaaacaggca | | | |
| | | | | ccccgtatatgctctacaaagattcctgtaatcgaaagagcaaccagcagaacc | | | |
| | | | | tgggaaccatcaaatgcagcaacctgtgcacagaaatagtggagtacaccag | | | |
| | | | | caaagatgaggttgctgtttgtaatttggcttccctggccctgaatatgtatgt | | | |
| SEMA3B | “sema domain, | 203071_at | + | gcccctggagtcgcggagaaagggccgtaaccggaggacccacgcccctga | 7869 | NM_004636 | SEQ ID |
| | immunoglobutin domain | | | gcctcgcgctgagcgggggccgcgcagcgcaacgcactggtgaccagactgt | | | NO: 120 |
| | (Ig), short basic domain, | | | ccccacgccgggaaccaagcaggagacgacaggcgagagaggagccag | | | |
| | secreted, (semaphorin) | | | acagaccctgaaaagaaggacgggttggggccgggcacattgggggtcacc | | | |
| | 3B/sema domain, | | | ggccgatggagacaccaaccgacaggccctggctgagggcagctgcgcggg | | | |
| | immunoglobulin domain | | | cttatttattaacaggataacccttgaatgtagcagccccgggagggcggcaca | | | |
| | (Ig), short basic domain, | | | ggtcgggcgcaggattcagccggagggaagggacggggaagccgagctcc | | | |
| | secreted, (semaphorin) | | | agagcaacgaccagggccgaggaggtgcctggagtgcccaccctgggaga | | | |
| | 3B” | | | cagaccccacctccttgggtagtgagcagtg | | | |
| SERPINE | “serine (or cysteine) | 202627s_at | | ggaactacggggcttacaggagcttttgtgtgcctggtagaaactatttctgttcca | 5054 | NM_000602 | SEQ ID |
| 1 | proteinase inhibitor, | | | gtcacattgccatcactcttgtactgcctgccaccgcggaggaggctggtgacag | | | NO: 121 |
| | clade E (nexin, plasmin- | | | gccnaaaggccagggaagaaaaccctttcatctcagagtccactgtggcact | | | |
| | ogen activator inhibitor | | | ggccacccctccccagtacaggggtgctgcaggtggcagagtgaatgtccccc | | | |
| | type 1), member 1” | | | atcatgtggcccaactctcctggcctggccatctccctccccagaaacagtgtgc | | | |
| | | | | atgggttamtggagtgtaggtgacttgtttactcattgaagcagatttctgcttcctttt | | | |
| | | | | atttttataggaatagaggaagaaangtcagatgcgtgcccagctcttcaccccc | | | |
| | | | | caatctcttggtggggaggggtgtacctaaatatttatcatatccttgcccttgag | | | |
| SERPINE | “serine (or cysteine) | 202628_s_at | | aaattgaccatacaatttcatcctccttcaggggatcaaaaggacggagtgggg | 5054 | NM_000602 | SEQ ID |
| 1 | proteinase inhibitor, | | | ggacagagactcagatgaggacagagtggtttccaatgtgttcaatagatttagg | | | NO: 122 |
| | clade E (nexin, plasmin- | | | agcagaaatgcaaggggctgcatgacctaccaggacagaactttccccaatta | | | |
| | ogen activator inhibitor | | | cagggtgactcacagccgcattggtgactcacttcaatgtgtcatttccggctgctg | | | |
| | type 1), member 1” | | | tgtgtgagcagtggacacgtgaggggggggtgggtgagagagacaggcagct | | | |
| | | | | cggattcaactaccttagataatatttctgaaaacctaccagccagagggta | | | |
| SLC20A1 | “solute carrier family 20 | 201920_at | | gtatcaggcttcaattccattatgttttaatgttgtctctgaagatgacttgtgattttttttt | 6574 | NM_005415 | SEQ ID |
| | (phosphate transporter), | | | cttttttttaaaccatgaagagccgtttgacagagcatgctctgcgttgttggtttcac | | | NO: 123 |
| | member 1/solute carrier | | | cagcttctgccctcacatgcacagggatttaacaacaaaaatataactacaactt | | | |
| | family 20 (phosphate | | | cccttgtagtctcttatataagtagagtccttggtactctgccctcctgtcagtagtgg | | | |
| | transporter), member 1” | | | caggatctattggcatattcgggagcttcttagagggatgaggttctttgaacaca | | | |
| | | | | gtgaaaatttaaattagtaacutmgcaagcagtuattgactgttattgctaagaa | | | |
| | | | | gaagtaagaaagaaaaagcctgttggcaatcttggttatttctttaagatttctggc | | | |
| | | | | agtgtgggatggatgaatgaagtggaatgtgaactttgggcaagttaaatggga | | | |
| | | | | cagccttccatgttcatttgtctacctcttaactga | | | |
| SPRY1 | “sprouty homolog 1, | 212558_at | | taattttagattcgacaatgtaaatcttcacattggagataatattggttggacc | 10252 | NM_005841/ | SEQ ID |
| | antagonist of FGF | | | ttgcccatcttcactctagccttcgtatttgtgaaggactcagccaccttccttcttcac | | NM_199327 | NO: 124 |
| | signaling (Drosophila)” | | | cccatgcttctcaccaaatttttgttgtcattgagggcacttggataactcaagttgat | | | |
| | | | | atttatagctgatcaatctatatgtgtcacagaactatgctgcctaaagtgatcttgg | | | |
| | | | | ctccttaatggtccttttggccccttggatagttaacagctgagtaattctaatctcttc | | | |
| | | | | tgtgttttccttgccttaaccacaaattgtgg | | | |
| SPRY2 | sprouty homolog 2 | 204011_at | | gagatacagaacttggtgacccatgtattgcataagctaaagcaacacagaca | 10253 | NM_005842 | SEQ ID |
| | (Drosophila)/sprouty | | | ctcctaggcaaagtttttgtttgtgaatagtacttgcaaaacttgtaaattagcagat | | | NO: 125 |
| | homolog 2 (Drosophila) | | | gacttttttccattgttttctccagagagaatgtgctatatttttgtatatacaataatattt | | | |
| | | | | gcaactgtgaaaaacaagttgtgccatactacatggcacagacacaaaatatta | | | |
| | | | | tactaatatgttgtacattcggaagaatgtgaatcaatcagtatgtttttagattgtatt | | | |
| | | | | ttgccttacagaaagcctttattgtaagactctgamcccmggacttcatgtatattg | | | |
| | | | | tacagttacagtaaaattcaacctttattttctaattttttcaacatattgtttagtgtaaa | | | |
| | | | | gaatatttatttgaagttttattattttataaaaaagaatatttattttaagaggcatctta | | | |
| | | | | caaattttgccccttt | | | |
| SRPUL | sushi-repeat protein/ | 205499_at | | gcggcatgtgaccatcattgaactggtgggacagccacctcaggaggtggggc | 27286 | NM_014467 | SEQ ID |
| | sushi-repeat protein | | | gcatccgggagcaacagctgtcagccaacatcatcgaggagctcaggcaattt | | | NO: 126 |
| | | | | cagcgcctcactcgctcctacttcaacatggtgttgattgacaagcagggtattga | | | |
| | | | | ccgagaccgctacatggaacctgtcacccccgaggaaatcttcacattcattgat | | | |
| | | | | gactacctactgagcaatcaggagttgacccagcgtcgggagcaaagggaca | | | |
| | | | | tatgcgagtgaacttgagccagggcatggttaaagtcaagggaaaagctcctct | | | |
| | | | | agttagctgaaactgggacctaataaaaggaggaaatgttttcccacagttctag | | | |
| | | | | ggacaggactctgaggtgggtgagtttgacaaatcctgcagtgtttccaggcatc | | | |
| | | | | cttttaggactgtgtaatagtttccctagaagctaggtagggactgaggacaggc | | | |
| | | | | cttgggcagtgggtt | | | |
| TCF8 | transcription factor 8 | 208078_s_at | + | agactgggcgaaaggctgtccggagggcagaccaggtgccttgccgcagag | 6935 | NM_030751 | SEQ ID |
| | (represses interleukin 2 | | | aaaacaccaaagtctcctgttcgctcataaagaagtttttgggatgggagagaat | | | NO: 127 |
| | expression)/transcription | | | ccagaccatcttggggcagccaggcccttgccttcatttttacagaggtagcaca | | | |
| | factor 8 (represses | | | actgattccaacacaaaaccccttcccctttttaaaatgatttctgttctaatgccata | | | |
| | interleukin 2 expression)/ | | | gatcaaaggcctcagaaaccattgtgtgtttcctctttgaagcaatgacaagcact | | | |
| | transcription factor 8 | | | ttactttcacggtggtttttgttttttcttattgctgtggaacctcttttggaggacgttaaa | | | |
| | (represses interleukin 2 | | | ggcgtgttttacttgtttttttaagagtgtgtgatgtgtgttttgtagatttcttgacagtgc | | | |
| | expression) | | | tgtaatacagacggcaatgcaatagcctatttaa | | | |
| TGFA | “transforming growth | 205016_at | | cctgccctctagttggttctgggctttgatctcttccaacctgcccagtcacagaag | 7039 | NM_003236 | SEQ ID |
| | factor, alpha/ | | | gaggaatgactcaaatgcccaaaaccaagaacacattgcagaagtaagaca | | | NO: 128 |
| | transforming growth | | | aacatgtatatttttaaatgttctaacataagacctgttctctctagccattgatttacc | | | |
| | factor, alpha” | | | aggctttctgaaagatctagtggttcacacagagagagagagagtactgaaaa | | | |
| | | | | agcaactcctcttcttagtcttaataatttactaaaatggtcaacttttcattatctttatt | | | |
| | | | | ataataaacctgatgcttttttttagaactccttactctgatgtctgtatatgttgcactg | | | |
| | | | | aaaaggttaatatttaatgtittaatttattttgtgtggtaagttaattttgatttctgtaatg | | | |
| | | | | tgttaatgtgattagcagttattttccttaatatctgaattatacttaaagagtagtgag | | | |
| | | | | caatataagacgcaattgtgtttttcagt | | | |
| TGFBR2 | “transforming growth | 208944_at | | gtttggatggtggaaggtctcattttattgagatttttaagatacatgcaaaggtttgg | 7048 | NM_003242 | SEQ ID |
| | factor, beta receptor II | | | aaatagaacctctaggcaccctcctcagtgtgggtgggctgagagttaaagaca | | | NO: 129 |
| | (70/80 kDa)/transforming | | | gtgtggctgcagtagcatagaggcgcctagaaattccacttgcaccgtagggca | | | |
| | growth factor, beta | | | tgctgataccatcccaatagctgttgcccattgacctctagtggtgagtttctagaat | | | |
| | receptor II (70/80 kDa)” | | | actggtccattcatgagatattcaagattcaagagtattctcacttctgggttatcag | | | |
| | | | | cataaactggaatgtagtgtcagaggatactgtggctt | | | |
| TIEG | TGFB inducible early | 202393_s_at | | tttgcctgcagtttcttgtgtagatttgaaaattgtataccaatgtgttttctgtagactct | 7071 | NM_005655 | SEQ ID |
| | growth response/TGFB | | | aagatacactgcactttgtttagaaaaaaaactgaagatgaaatatatattgtaa | | | NO: 130 |
| | inducible early growth | | | agaagggatattaagaatcttagataacttcttgaaaaagatggcttatgtcatca | | | |
| | response | | | gtaaagtacctttatgttatgaggatataatgtgtgctttattgaattagaaaattagt | | | |
| | | | | gaccattattcacaggtggacaaatgttgtcctgttaatttataggagttttttgggga | | | |
| | | | | tgtggaggagttgggtagaaaaattattagaacattcacttttgttaacagtatttct | | | |
| | | | | cttttattctgttatatagtggatgatatacacagtggcaaaacaaaagtacattgct | | | |
| | | | | taaaatatatagtgaaaaatgtcactatatcttcccatttaacattgtttttgtatattgg | | | |
| | | | | gtgtagatttctgacatcaaaacttggacccttggaaaacaaaagttttaattaaa | | | |
| | | | | aaaaatccttgtgacttacaatttgcac | | | |
| TIMP3 | “tissue inhibitor of | 201147_s_at | | gagtcggagatgatgcagcacacacacaattccccagcccagtgatgcttgtgt | 7078 | NM_000362 | SEQ ID |
| | metalloproteinase 3 | | | tgaccagatgttcctgagtctggagcaagcacccaggccagaataacagagct | | | NO: 131 |
| | (Sorsby fundus dystrophy, | | | ttcttagttggtgaagacttaaacatctgaggtcaggaggcaatttgcctgcc | | | |
| | pseudoinflammatory)” | | | ttgtacaaaagctcaggtgaaagactgagatgaatgtctttcctctccctgcctccc | | | |
| | | | | accagacttcctcctggaaaacgcutggtagatttggccaggagctttcttttatgt | | | |
| | | | | aaattggataaatacacacaccatacactatccacagatatagccaagtagattt | | | |
| | | | | gggtagaggatactatttccagaatagtgtttagctcacctagggggatatgtttgt | | | |
| | | | | atacacatttgcatatacccacatgggg | | | |
| TIMP3 | “tissue Inhibitor of | 201148_s_at | | ttgttgtcgttgcttgtttgaagaaaatcatgacattccaagttgacattttttttttcatttt | 7078 | NM_000362 | SEQ ID |
| | metalloproteinase 3 | | | aattaaaatttgaaattctgaacaccgtcagcaccctctcttccctatcatgggtcat | | | NO: 132 |
| | (Sorsby fundus dystrophy, | | | ctgacccctgtccgtctccttgtccctgcttcatgtttgggggcctttctttaactgcctt | | | |
| | pseudoinflammatory)” | | | cctggcttagctcagatggcagatgagagtgtagtcaagggcctgggcacagg | | | |
| | | | | agggagagctgcagagtgtcctgcctgccttggctggagggacacctctcctgg | | | |
| | | | | gtgtggagacagcttggttccctttccctagctccctggtgggtgaatgccacctcc | | | |
| | | | | tgagatcctcacctcttggaattaaaattgttggtcactggggaaagcctgagtttg | | | |
| | | | | caaccagttg | | | |
| TIMP3 | “tissue inhibitor of | 201149_s_at | | aggggctgaactatcggtatcacctgggttgtaactgcaagatcaagtcctgcta | 7078 | NM_000362 | SEQ ID |
| | metalloproteinase 3 | | | ctacctgccttgctttgtgacttccaagaacgagtgtctctggaccgacatgctctc | | | NO: 133 |
| | pseudoinflammatory)/ | | | caatttcggttaccctggctaccagtccaaacactacgcctgcatccggcagaa | | | |
| | tissue inhibitor of | | | gggcggctactgcagctggtaccgaggatgggcccccccggataaaagcatc | | | |
| | metalloproteinase 3 | | | atcaatgccacagacccctgagcgccagaccctgccccacctcacttccctccc | | | |
| | (Sorsby fundus dystrophy, | | | ttcccgctgagcttcccttggacactaactcttcccagatgatgacaatgaaattag | | | |
| | pseudoinflammmatory)” | | | tgcctgttttcttgcaaatttagcacttggaacatttaaagaaaggtctatgctgtcat | | | |
| | | | | atggggtttattgggaactatcctcctggcc | | | |
| TIMP3 | “tissue inhibitor of | 201150_s_at | | gactttttggaatagccctgtctagggcaaactgtggcccccaggagacactacc | 7078 | NM_000362 | SEQ ID |
| | metalloproteinase 3 | | | cttccatgccccagacctctgtcttgcatgtgacaattgacaatctggactacccc | | | NO: 134 |
| | (Sorsby fundus dystrophy, | | | aagatggcacccaagtgtttggcttctggctacctaaggttaacatgtcactagag | | | |
| | pseudoinflammatory)” | | | tatttttatgagagacaaacattataaaaatctgatggcaaaagcaaaacaaaat | | | |
| | | | | ggaaagtaggggaggtggatgtgacaacaacttccaaattggctctttggaggc | | | |
| | | | | gagaggaaggggagaacttggagaatagtttttgctttgggggtagaggsttctt | | | |
| | | | | agattctcccagcatccgcctttccctttagccagtctgctgtcctgaaacccagaa | | | |
| | | | | gtgatggagagaaaccaacaagagatctcgaaccctgtctagaaggaatgtat | | | |
| | | | | ttgttgctaaatttcgtagcactgtttacagttttcctccatgttatttatg | | | |
| TNFRSF6 | “tumor necrosis factor | 204781_s_at | + | gagtattactagagcttgccacctctccatttttgccttggtgctcatcttaatggcct | 355 | NM_000043/ | SEQ ID |
| | receptor superfamily, | | | aatgcacccccaaacatggaaatatcaccaaaaaatacttaatagtccaccaa | | NM_152871/ | NO: 135 |
| | member 6” | | | aaggcaagactgcccttagaaattctagcctggtttggagatactaactgctctca | | NM_152872/ | |
| | | | | gagaaagtagcttgtgacatgtcatgaacccatgtttgcaatcaaagatgataa | | NM_152873/ | |
| | | | | aatagattcttatttttcccccacccccgaaaatgttcaataatgtcccatgtaaaac | | NM_152874/ | |
| | | | | ctgctacaaatggcagcttatacatagcaatggtaaaatcatcatctggatttagg | | NM_152875/ | |
| | | | | aattgctcttgtcatacccccaagtttctaagatttaagattctccttactactatccta | | NM_152876/ | |
| | | | | cgtttaaatatctttgaaagtttgtattaaatgtgaattttaagaaataatatttatatttc | | NM_152877 | |
| | | | | tgtaaatgtaaactgtgaagatagttataaactgaagcagatacctggaaccac | | | |
| | | | | ctaaagaacttccatttatggaggatttttttgccccttgtgtttggaattat | | | |
| TNFRSF6 | “tumor necrosis factor | 215719_x_at | + | accaaggttctcatgaatctccaaccttaaatcctgaaacagtggcaataaattta | 355 | NM_000043/ | SEQ ID |
| | receptor superfamily, | | | tctgatgttgacttgagtaaatatatcaccactattgctggagtcatgacactaagtc | | NM_152871/ | NO: 136 |
| | member 6” | | | aagttaaaggctttgttcgaaagaatggtgtcaatgaagccaaaatagatgaga | | NM_152872/ | |
| | | | | tcaagaatgacaatgtccaagacacagcagaacagaaagttcaactgcttcgt | | NM_152873/ | |
| | | | | aattggcatcaacttcatggaaagaaagaagcgtatgacacattgattaaagat | | NM_152874/ | |
| | | | | ctcaaaaaagccaatctttgtactcttgcagagaaaattcagactatcatcctcaa | | NM_152875/ | |
| | | | | ggacattactagtgactcagaaaattcaaacttcagaaatgaaatccaaagctt | | NM_152876/ | |
| | | | | ggtctagagtgaaaaacacaaattcagttctgagtatatgcaattagtgtttgan | | NM_152877 | |
| | | | | aagattcttaatagctggctgtaaatactgcttggttt | | | |
| TNFSF10 | “tumor necrosis factor | 202687_s_at | + | gtagcagctcacataactgggaccagaggaagaagcaacacattgtcttctcc | 8743 | NM_003810 | SEQ ID |
| | (ligand) superfamily, | | | aaactccaagaatgaaaaggctctgggccgcaaaataaactcctgggaatcat | | | NO: 137 |
| | member 10” | | | caaggagtgggcattcattcctgagcaacttgcacttgaggaatggtgaactggt | | | |
| | | | | catccatgaaaaagggttttactacatctattcccaaacatactttcgatttcagga | | | |
| | | | | ggaaataaaagaaaacacaaagaacgacaaacaaatggtccaatatatttac | | | |
| | | | | aaatacacaagttatcctgaccctatattgttgatgaaaagtgctagaaatagttgt | | | |
| | | | | tggtctaaagatgcagaatatggactctattccatctatcaagggggaatatttga | | | |
| | | | | gcttaaggaaaatgacagaatttttgtttctgtaacaaatgagcacttgatagacat | | | |
| | | | | ggaccatgaagccagttttttcggggcctttttagttggctaactgacct | | | |
| TNFSF10 | “tumor necrosis factor | 202688_at | + | ctctacctcatatcagtttgctagcagaaatctagaagactgtcagcttccaaaca | 8743 | NM_003810 | SEQ ID |
| | (ligand) superfamily, | | | ttaatgcaatggttaacatcttctgtctttataatctactccttgtaaagactgtagaa | | | NO: 138 |
| | member 10/tumor | | | gaaagcgcaacaatccatctctcaagtagtgtatcacagtagtagcctccaggtt | | | |
| | necrosis factor (ligand) | | | tccttaagggacaacatccttaagtcaaaagagagaagaggcaccactaaaa | | | |
| | superfamily, member 10” | | | gatcgcagtttgcctggtgcagtggc | | | |
| TUBB4 | “tubulin, beta, 4” | 213476_x_at | | gctcacccagcagatgttcgatgccaagaacatgatggccgcctgcgacccgc | 10381 | NM_006086 | SEQ ID |
| | | | | gccacggccgnctanctgacggtggccaccgtgttccggggccgcatgtccat | | | NO: 139 |
| | | | | gaaggaggtggacgagcagatgctggccatccagagcaagaacagcagcta | | | |
| | | | | cttcgtggagtggatccccaacaacgtgaaggtggccgtgtgtgacatcccgcc | | | |
| | | | | ccgcggcctcaagatgtcctccaccttcatcgggaacagcacggccatccagg | | | |
| | | | | agctgttcaagcgcatctccgagcagttcacggccatgttccggcgcaaggcctt | | | |
| | | | | cctgcactggtacacgggcgagggcatggacgagatggagttcaccgaggcc | | | |
| | | | | gagagcaacatgaacgacctggtgtccgagtacc | | | |
| TUSC3 | tumor suppressor | 209227_at | | gatgcctaaccaaggactagagctccttcttgagatctaaatctaaagtaaatgtg | 7991 | NM_006765/ | SEQ ID |
| | candidate 3 | | | cattaaagcagtgtgcttcaaaggcatcagacgatgaaagcaacataccacaa | | NM_178234 | NO: 140 |
| | | | | ctaggagttatttctcaaacttaaatgtcctctgggaatccagacttaaaaataag | | | |
| | | | | agcaaacttaacacactatccattttcgagcaaacttaacccactatatccattttg | | | |
| | | | | ctcatgtgttttatgcaaccagctttccatcaaatcctcaatccttgaatccaggtaa | | | |
| | | | | aaggttaattatcctaggattagtgaatgattcaatgaagctttcttgaaaacaaac | | | |
| | | | | ataggagtgtaatgtactattatgtttgtatcctgttttagtttataaagcactttcacat | | | |
| | | | | acattatgg | | | |
| TUSC3 | tumor suppressor | 209228_x_at | | acccaactactctggtaccattgctttggccctgttagtgtcgcttgttggaggtttgc | 7991 | NM_006765/ | SEQ ID |
| | candidate 3 | | | tttatttgagaaggaacaacttggagttcatctataacaagactggttgggccatg | | NM_178234 | NO: 141 |
| | | | | gtgtctctgtgtatagtctttgctatgacttctggccagatgtggaaccatatccgtgg | | | |
| | | | | acctccatatgctcataagaacccacacaatggacaagtgagctacattcatgg | | | |
| | | | | gagcagccaggctcagtttgtggcagaatcacacattattctggtactgaatgcc | | | |
| | | | | gctatcaccatggggatggttcttctaaatgaagcagcaacttcgaaaggcgatg | | | |
| | | | | ttggaaaaagacggataatttgcctagtgggattgggcctggtggtcttcttcttca | | | |
| | | | | gttttctactttcaatatttcgttccaagtaccacggctatccttatagtgatctggactt | | | |
| | | | | tgagtgagaagatgtgatttggaccatggcacttaaaaactctataacctcagcct | | | |
| | | | | tttaat | | | |
| TUSC3 | tumor suppressor | 213423_x_at | | ctttgctatgacttctggccagatgtggaaccatatccgtggacctccatatgctcat | 7991 | NM_006765/ | SEQ ID |
| | candidate 3 | | | aagaacccacacaatggacaagtgagctacattcatgggagcagccaggctc | | NM_178234 | NO: 142 |
| | | | | agtttgtggcagaatcacacattattctggtactgaatgccgctatcaccatgggg | | | |
| | | | | atggttcttctaaatgaagcagcaacttcgaaaggcgatgttggaaaaagacgg | | | |
| | | | | ataatttgcctagtgggattgggcctggtggtcttcttcttcagttttctactttcaatattt | | | |
| | | | | cgttccaagtaccacggctatccttatagctttttaattaaatgaagccaagtggga | | | |
| | | | | tttgcataaagtgaatgtttaccatgaagataaactgttcctgactttatactattttga | | | |
| | | | | attcattcatttcattgtgatcagctagcttattcttgtgtac | | | |

### EXAMPLE 4

### RT-PCR Confirmation Studies

In addition, the sequence of the RT-PCR primers used in the confirmatory follow up studies as highlighted in FIGS. 3, 4, 5 and 6 are listed in Table 3. Note that DAPK2 was not identified by Affymetrix analysis, only via follow up of the DAPK gene family by RT-PCR following discovery of predictivity of DAPK1. Hence no Affymetrix ID or Affymetrix ID sequence is provided for DAPK2.

TABLE 3

Sequences relevant to genes followed up by RT-PCR (see FIGS. 3, 4, 5 & 6)
(all sequences written 5′-3′)

| | | | | | |
|---|---|---|---|---|---|
| | | | Taqman | Taqman | |
| | | | Forward | Reverse | Taqman |
| Gene | affy Id | affy probe seq | Primer | Primer | probe |

| | | | | | |
|---|---|---|---|---|---|
| EMP1 | 201324_at | CACCAAATTACCTAGGCTGAGGTTAGAGAGATTGGCCAGCAAA | AGCCATCCTG | ACCTTACAAAC | CAAAGCA |
| | | AACTGTGGGAAGATGAACTTTGTCATTATGATTTCATTTATCAC | CCCTTCTGA | TCTCTTTCC | AAACATC |
| | | ATGATTATAGAAGGCTGTCTTAGTGCAAAAAACATACTACATT | | | ACATTCC |
| | | TCAGACATATCCAAAGGGAATACTCACATTTTTGTTAAGAAGTT | | | AGTC |
| | | GAACTATGACTGGAGTAAACCATGTATTCCCTTATCTTTTACTT | | | |
| | | TTTTTCTGTGACATTTATGTCCTCATGTAATTTGCATTACTCTG | | | |
| | | GTGGATTGTTCTAGTACTGTATTGGGCTTCTTCGTTAAT | | | |
| NES | 218878_at | GCAGCACTCTTAACTTACGATCTCTTGACATACGGTTTCTGGC | GCCCCTTTCA | AGTGCCGGGG | AGTGCTC |
| | | TGAGAGGCCTGGCCCGCTAAGGTGAAAAGGGGTGTGGGCAA | GGAGGAGGA | AGATGGTCTT | TGAAGAC |
| | | AGGAGCCTACTCCAAGAATGGAGGCTGTAGGAATATAACCTC | | | CTCTGG |
| | | CCACCCTGCAAAGGAATCTCTTGCCTGCTCCATTCTCATAGG | | | GC |
| | | CTAAGTCAGCTGAATCCCGATAGTACTAGGTCCCCTTCCCTCC | | | |
| | | GCATCCCGTCAGCTGGAAAAGGCCTGTGGGCCCAGAGGCTTC | | | |
| | | TCCAAAGGGAGGGTGACATGCTGGCTTTTGTGCCCAAGCTCA | | | |
| | | CCAGCCCTGCGCCACCTCACTGCAAGTAGTGCACCATCTCAC | | | |
| | | TGCAGTAGCACGCCCTCCTGGGCCGTCTGGCCTGTGGCTAAT | | | |
| | | GGAGGTGACGGCACTCCCATTGTGCTGACTCCCCCCATCCCT | | | |
| | | GCCACGCTGTGGCCCTGCCTGGCTAGTCCCTGCCTGAATAAA | | | |
| | | G | | | |
| DAPK1 | 203139_at | CCTCCTGCAGGGTGATTTTATGATCAGTGTTGTTGCTCTAGGA | AGGAAACGCT | CTGGAGGAGG | CTTGCTG |
| | | AGACATTTTTCCGTTTGCTTTTGTTCCAATGTCAATGGTGAACG | ACCTCTCTGT | ATCCCTTCT | TATGCTG |
| | | TCCACATGAAACCTACACACTGTCATGCTTCATCATTCCCTCTC | | | ATCATCG |
| | | ATCTCAGGTAGAAGGTTGACACAGTTGTAAGGGTTACAGAGAC | | | CC |
| | | CTATGTAAGAATTCAGAAGACCCCTGACTCATCATTTGTGGCA | | | |
| | | GTCCCTTATAATTGGTGCATAGCCAGATGGTTTCCACATTTAG | | | |
| | | ATCCTGGTTTCATAACTTCCTGTACTTGAAGTCTAAAAGCAGAA | | | |
| | | AATAAAGGAAGCAAGTTTTTCTTCCATGATTTTAAATTGTGATC | | | |
| | | GAGTTTTAAATTGATAGGAGGGAACATGTCCTAATTCTTCTGT | | | |
| | | CCTGAGAA | | | |
| DAPK2 | Not | Not applicable | GGGTAGGCAC | AGTGCAGTGG | TACTCCA |
| | applicable | | CTGGCATC | CGTGATCTC | GGGGCT |
| | | | | | GAGGTGA |
| | | | | | CA |

### EXAMPLE 5

### Diagnostic Test for Clinical Studies

The predictive gene lists above have been generated using the preclinical studies described. The following approach is employed to develop a diagnostic test for the clinical setting based on this data.

a) Identify patients which represent the population of individuals whom we would expect to derive benefit from a diagnostic test, and for which pre-treatment tumour samples and outcome of gefitinib treatment are known or will be available. For each sample the expression level for our genes of interest is evaluated, using for example the RNA signal from RT-PCR. QC procedures are applied to identify the set of samples and genes to take forward to step b).
a) Identify a subset of the genes which together are able to distinguish between patients showing different responses to gefitinib. There are a variety of methods which are useful to select the subset of genes and combine their expression values to provide a prediction, possibly a predictive value and a corresponding threshold which distinguishes between different patient groups. An example is stepwise Linear Discrminant Analysis where genes that distinguish well between patient groups are successively added to a linear combination until addition of a further gene does not provide additional predictive power (Mardia et al.). The threshold value of the linear combination is then selected to give the appropriate sensitivity and specificity properties.
d) Tool validation would partly be carried out during development in step 2, for example using cross validation and permutation tests. In addition, the finally developed diagnostic procedure (gene subset and method of combining to generate a prediction and a platform for biological analysis) is tested and validated in its entirety using an independent set of samples not used within tool development in step b).

### REFERENCES

Bailey et al Lung Cancer (2003) 41 S2, S71
Downward et al. (1984) Nature, 307, p 521
Fukuoka et al (2003) J. Clin. Oncol., 21, p 2237
Kris et al. (2003) JAMA, 290, p 2149
Lynch et al (2004) New England Journal of Medicine, 350(21) p 2129
Mardia K. V., Kent J. T., Bibby J. M. (1979) “Multivariate Analysis” London, Academic Press Inc. Ltd.
Paez et al. (2004) Science, 304 p
Salomon et al. (1995) Crit. Rev. Oncol. Haematol, 19, p 183
Scheffe, H. (1959) “The Analysis of Variance” New York, Wiley
Sporn & Todaro (1980) New England Journal of Medicine 303, p 878
Storey (2003) “Statistical Significance for Genome Wide Studies” PNAS, vol 100, issue 16, pp 9440-9445
Yarden & Sliwkowski (2001) Nature Reviews Molecular Cell Biology, 2, p 127

1. A method of selecting a mammal having or suspected of having a tumour for treatment with an erbB receptor drug which comprises testing a biological sample from the mammal for expression of any one of the genes listed in Table 1 or DAPK2, whereby to predict an increased likelihood of response to the erbB receptor drug.

2. A method according to claim 1 comprising testing a biological sample from the mammal for expression of any one of NPAS2, NES, CHST7, DAPK1, ACOX2, GSPT2, TNNC1 or DAPK2.

3. A method according to claim 1 comprising testing a biological sample from the mammal for expression of any one of NPAS2, NES, CHST7 or DAPK1.

4. A method according to claim 1 comprising testing a biological sample from the mammal for expression of at least two of NPAS2, NES, CHST7 or DAPK1.

5. A method according to claim 1 comprising testing a biological sample from the mammal for expression of at least three of NPAS2, NES, CHST7 or DAPK1.

6. A method according to claim 1 comprising testing a biological sample from the mammal for expression of NPAS2, NES, CHST7 and DAPK1.

7. A method according to claim 1 additionally comprising testing a biological sample from the mammal for expression of any gene listed in Table 2 as defined herein.

8. A method according to claim 7 comprising testing a biological sample from the mammal for expression of any one of EMP1, SLC20A1, SPRY2 or PGM1.

9. A method according to claim 7 comprising testing a biological sample from the mammal for expression of EMP1.

10. A method according to claim 1 wherein the tumour is selected from the group consisting of leukaemia, multiple myeloma, lymphoma, bile duct, bone, bladder, brain, CNS, glioblastoma, breast, colorectal, cervical, endometrial, gastric, head, neck, hepatic, lung, muscle, neuronal, oesophageal, ovarian, pancreatic, pleural membrane, peritoneal membrane, prostate, renal, skin, testicular, thyroid, uterine and vulval.

11. A method according to claim 10 wherein the tumour is selected from one of non-small cell lung, pancreatic, head or neck.

12. A method according to claim 1 wherein the erbB receptor drug is selected from any one of gefitinib, erlotinib, PKI-166, EKB-569, HKI-272, lapatinib, canertinib, AEE788, XL647, BMS 5599626, cetuximab, matuzumab, panitumumab, MR1-1, IMC-11F8 or EGFRL11.

13. A method according to claim 12 wherein the erbB receptor drug is gefitinib.

14. A method according to claim 1 wherein the mammal is a human and in which the method comprises testing a biological sample from the human for increased expression of DAPK1 and decreased expression of NPAS2, NES, CHST7 and EMP1 whereby to predict an increased likelihood of response to gefitinib.


SEQUENCE LISTING (142 sequences)

SEQ ID NO: 1 - DNA, 425 bp, Homo sapiens
gtgcagcatttacagaccctgacgcaatccggagctgaccagcacgaggcttggaaccagaccactgtcatacacctccaggctgctaaggtgcactgctactatgtcactgtgaagggttttacagaagctctggagaaactagaaaatgaaccagcgattcagcaggtgctcaagcgcctctgtgacctccatgccatacatggaatcttgactaactcgggtgactttctccatgacgccttcctgtctggtgcccaagtggacatggcaagaacagcctacctggacctgctccgcctgatccggaaggatgccatcctgttaactgatgcttttgacttcaccgatcagtgtttaaattcagcccttggctgttatgatggaaacgtctacgaacgcctgttccagtgggctcagaagtc

SEQ ID NO: 2 - DNA, 507 bp, Homo sapiens
gagcttaagatctggtgttttgttaatgcttctgtttattccagaagcattaaggtaacccattgccaagtatcattcttgcaaattattcttttatataactgaccagtgcttaataaaacaagcaggtacttacaaataattactggcagtaggttataattggtggtttaaaaataacattggaatacaggacttgttgccaattgggtaattttcattagttgttttgtttgttttgatttgaaacctggaaatacagtaaaatttgactgtttaaaatgttggccaaaaaaatcaagatttaatttttttatttgtactgaaaaactaatcataactgttaattctcagccatctttgaagcttgaaagaagagtctttggtattttgtaaacgttagcagactttcctgccagtgtcagaaaatcctatttatgaatcctgtcggtattccttggtatctgaaaaaaataccaaatagtaccatacatgagttatttctaa

SEQ ID NO: 3 - DNA, 348 bp, Homo Sapiens
agaatagagaggaggcttgaaggaaccagcaatgagaaggccaggaaaagaaagagctgaaaatggagaaagcccaagagttagaacagttggatacaggagaagaaacagcggctccactacagacccagccccaggttcaatgtcctccgaagaatgaagtctttccctggtgatggtcccctgccctgtctttccagcatccactctcccttgtcctcctgggggcatatctcagtcaggcagcggcttcctgatgatggtcgttggggtggttgtcatgtgatgggtccctccaggttactaaagggtgcatgtcccctgcttgaacactgaagggcaggtggt

SEQ ID NO: 4 - DNA, 487 bp, Homo Sapiens
aactcatacgtcctgtggtggcattgggagagttcccccatgatgagggccaagatagaatctgtaccactcagtgctaccatccccacccctacaccacttccacacaggggcctcatggcatggtcagggtcccagctgtaggtgagagcagggcactgtccagctgtccactggggaagtcaagatgtcctaaggcccaggtcagggcatctggagtctgaaggaccctagttcctagaggcatctggcagcaagaaggtgaggcatcagggaacgggaatcaggctgggactgatcagaggtgaagggacagagagaggagaggaggaagattgagctgggggcaacagccaagctcacctgggcaggtctctgccacctccttgctctgtgagctgtcagtctaggttattctctttttttgtggctatttttaattgctttggatttgttaaatgttttctgtcttctgttaagtgtgttt

SEQ ID NO: 5 - DNA, 318 bp, Homo Sapiens
tgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcagttactgtttttttaatcccctgaagttgtcctgtaggagacagaaattctttgctgtctgtatcccttggagtaa

SEQ ID NO: 6 - DNA, 135 bp, Homo Sapiens
gaggcaaatggatctcgatatttcagatgggcttttgatgcactgttgccaaggaaggctttttctgattttttgacaaatgaatttttgcacactttcattggtgtctttcggcaacttacacacattgaaaat

SEQ ID NO: 7 - DNA, 402 bp, Homo Sapiens
caagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagt

SEQ ID NO: 8 - DNA, 417 bp, Homo Sapiens
attgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacaccc

SEQ ID NO: 9 - DNA, 546 bp, Homo Sapiens
  misc_feature (104)..(104): n is a, c, g, or t
ttctatgcatccacaccaaaatcctgcagaatgtaagtaagctctgctttataagatgggttcaccttcatcgcagactgaaagtttcagtttttatttttttncagaaagcacgaaaaattatttataatagtctggagaaaaaacacactgtaatatttcaagtgtatgcagtagaatgtactgtaactgagccctttcccacatgtctaggctccaatgtctcctgtaggtccacctaactgtgtgttttcagggacaatgccatccatgtttgtgctgtagacttgctgctgctgaatcctttctggggactttctcatcgggcagggagcagagggcttctcgttcatgcaccctttgcctgaacacccatgtagctgctgtgttgtgtatatattactcttaagaggagtgtgtgtgtctgtgtttgttttaaaagtcacttatttcttacagtgatttcaattgcaccatgacttcttcactaaaaccacaaagtcctgcttaaaactatggaaaacctaacctgattagagccttgac

SEQ ID NO: 10 - DNA, 546 bp, Homo Sapiens
ggcaatctgtcacactctcagagtctgggacttgacttgctaccaacaactgctgtgcaattctgctgagcaggaatatcatgagctgttcaataatgacggacgcattggttgagatgaagtttccagtaaggaagtgacagtgcaatgtggatatttatggctgtaaaataggaagagctttagttcccaggctgaacctgccactgctggagccatttcaacaaggcatcctcacaacaaagaagagatgtgatttggtaccatttcacaccagcaggtgtctggacgaaaacatcaatgtgaataagggccaagtgcagtcctgtcttgattaaattacttaataatattattaaataataataggtctgggcagtattgtttttaacctgactcatccagctgtccttcaaatagctccgtctccctctacccagaactgatttttaaaaagaagtaatttttctccctgggctgggaaaaccctaatgaactgaaacacacttttactttaaaatttttctgtctggcgtttttgtaatc

SEQ ID NO: 11 - DNA, 496 bp, Homo Sapiens
gaattccctagaaatcctactgggaagtataggcagatctctccctcatataacggatgtttcttggcgcttggaatatcagataaagaccaatcaacttcataggatgtacagacctgcatatttggtgaccttaagtgtacagaacactgattccccatcctatccagagattagttttagttgcagcatggaacaattacaggacttggtggggaaacttaaagatgcttcgaaaagcctggaaagagcaactcagttgtaacttggggaagttaacgatccgcccgagtgcagaggaaaaccagaaacgccttgccttcagctgaaccaccgtttgtgcgagctggatgtccttttcagtagaaaagaattttccttttgaatttataccattcatcaattttgacactttaaaaacgtgtgaaagggttaagagggaaagatactgcccaagtatttgaatcgtttagtagtaactgtccatttatcctat

SEQ ID NO: 12 - DNA, 313 bp, Homo Sapiens
  misc_feature (190)..(190): n is a, c, g, or t
tataatacttcagtaaggcctttaaaaaatccacagtgatattattactcctaacaaaaacaataattacttagtatcatctaatatgtggttcatatttaaatttgttgttttgagatgggtcttacaattggtttattcaattgcattttttctaactcgtgtctcaagtgttttaaaaatctactgnacttataatgacttatataatgtatttctcattttacctttcttccaaaagaggaaataatggcaaaccatataatattgtacattcactgtcaaaaagcaaacccttgttttgataacttgt

SEQ ID NO: 13 - DNA, 395 bp, Homo Sapiens
cctcctccagggtgattttatgatcagtgttgttgctctaggaagacatttttccgtttgcttttgttccaatgtcaatgtgaacgtccacatgaaacctacacactgtcatgcttcatcattccctctcatctcaggtagaaggttgacacagttgtagggttacagagacctatgtaagaattcagaagacccctgactcatcatttgtggcagtcccttataattggtgcatagcagatggtttccacatttagatcctggtttcataacttcctgtacttgaagtctaaaagcagaaaataaaggaagcaagttttcttccatgattttaaattgtgatcgagttttaaattgataggagggaacatgtcctaattcttctgtcctgagaa

SEQ ID NO: 14 - DNA, 569 bp, Homo Sapiens
aggagaggatttgccactgcttttctaaggacgagaagcctgttgaagctattagggtttgttctgaagttttacagatggaacctgacaatgtgaatgccctgaaagatcgagctgaggcctatttgatagaggaaatgtatgatgaagctattcaggattatgaaactgctcaggaacacaatgaaaatgatcagcagattcgagaaggtctagagaaagcacaaagattattgaaacagtcgcagaaacgagattattataaaatcttgggagtaaaaagaaatgccaaaaagcaagaaattattaaagcataccgaaaattagcactgcagtggcacccagataacttccagaatgaagaagaaaagaaaaaagctgagaaaaagttcattgatatagcagctgctaaagaagtcctctctgatccagaaatgagaaagaagtttgacgacggagaagatcctttggatgcagagagccagcaaggaggcggcggcaaccctttccacagaagctggaactcatggcaagggttcaatcccttcagctcaggcggaccatttaga

SEQ ID NO: 15 - DNA, 481 bp, Homo Sapiens
tgagggccacgggcttgggtagtggaaagggtgtttgggaaattgttaaatcagttacccgtagtagagctatttcttgtacttctaagttttctagaagtggaaggattgtagtcatcctgaaaatgggtttacttcaaaatccctcagccttgttcttcacgactgtctatactgagagtgtcatgtttccacaaagggctgacacctgagcctggattttcactcatccctgagaagccctttccagtagggtgggcaattcccaacttccttgccacaagcttcccaggctttctcccctggaaaactccagcttgagtcccagatacactcatgggctgccctgggcagccagcattcattgtaagttccctctttgaaaactggtgtgtgggtgttcagttctgtgtctggtgggtatggacagacagtaatctcctgtgatctgtgctagctgtgaggcagctctggaacgtga

SEQ ID NO: 16 - DNA, 398 bp, Homo Sapiens
ggctcccagcaagggtaggacgggccgcatgcgggcagaaagttgggactgagcagctgggagcaggcgaccgagctccttccccatcatttctccttggccaacgacgaggccagccagaatggcaataaggactccgaatacataataaaagcaaacagaacactccaacttagagcaataacggctgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccagaagtgcctggttctg

SEQ ID NO: 17 - DNA, 499 bp, Homo Sapiens
gatacgctggggcccatgcagaaggagctggccgagcagctgggcctgtctactggcgagaaggagaagctgccgggagagctagagccggtgcaggccacgcagaacaagacagggaagtatgtgccgccgagcctgcgcgacggggccagccgccgcggggagtccatgcagcccaaccgcagagccgacgacaacgccaccatccgtgtcaccaacttgtcagaggacacgcgtgagaccgacctgcaggagctcttccggcctttcggctccatctcccgcatctacctggctaaggacaagaccactggccaatccaagggctttgccttcatcagcttccaccgccgcgaggatgctgcgcgtgccattgccggggtgtccggctttggctacgaccacctcatcctcaacgtcgagtgggccaagccgtccaccaactaagccagctgccactgtgtactcggtccgggacccttggcgacagaagacagcc

SEQ ID NO: 18 - DNA, 261 bp, Homo Sapiens
  misc_feature (41)..(42): n is a, c, g, or t
atgtgtcggggagagagcccgcagggaagggtaaagcccannggggcagggccctcccagatgcctgaggagggggcaggtcccctcccctctcctcctcttccctccccatctaaaggggtttggggagagacacaggcaggcgagggggctggtccccagtctgttggggtggtgctcagggtaaagggctatnggcaacaggggaccagaccagggatgagtggggagggcacaaggaccatttgccagaatccaccg

SEQ ID NO: 19 - DNA, 526 bp, Homo Sapiens
ctgttgctccaggatgcattctgataggagggggcggcagggctgggccttgtgacaatctgcctttcaccacatggccttgcctcggtggccctgactgtcagggagggccagggaggcagagcgggagggagtctcaggaggaggcttgccctgaggggctggggagggggtacctcatgaggaccagggtggagcttgagaagaggaggaggtgggggcttggaggtgcttggtagctgaggggacgggcaagtgagaggggagggagggaagtcctgggaggatcctgagctgctgttgcagtctaacccactaatcagttcttagattcaggggaagggcaggcaccaacaactcagaatgggggctttcggggagggcgcctagtccccccagctctaagcagccaggagggacctgcatctaagcatctgggttgccatggcaatggcatgccccccagctactgtatgcccccgacccccgcagaggcagaatgaacccatagggagctgatcgtaat

SEQ ID NO: 20 - DNA, 516 bp, Homo Sapiens
gggaccacctctatagtgatctggcggatctcatgctgcggcacggctggcgcacaggcgccatcatccccgagctggagcgtgagatccgcatcatcaacacggagcagtacatgcactcgctgacgtggcagcaggcgctcacggggctgctggagcgcatgcagacctatcaggacgcggagtcgaggcaggtgctggctgcctggatgaaagagcggcaggagctgaggtgcatcaccaaggccctgttcaatgcgcagttcggcagcatcttccgcaccttccacaaccccacctacttctcaaggcgcctcgtgcgcttctctgacctctacatggcctccctcagctgcctgctcaactaccgcgtggacttcaccttctacccacgccgtacgccgctgcagcacgaggcacccctctggatggaccagctctgcaccggctgcatgaagacccccttccttggtgacatggcccacatccgctgagggcacctttattgtctgggac

SEQ ID NO: 21 - DNA, 482 bp, Homo Sapiens
tattcaaacggagtcctcccattccaagaaactggaaacccctagtttatgttaaaaggccagtctaaattctttcacttacatctttacagaaaactatattttctctcttccatacccagaaatctaatcagaaaactgacttttctcatgttcaactggacctaggggaatatgacagaaaagcatcccataggctttaatatactttttaaaatatataaaactgaaaattaatagccatttaccctgaaagagttctgcgtggactttgtcacttgcatagtaatagcatgtgcctcattgttcagaagattagctttaggtcctattttcaaatacgaaatggtagcataagctgtaaaactgtagtcttctctgcagaaaataaaggccaacaataagaaagcttttgaaggaatcacggaaaacaaatttataaaagaaataactatatgcgcagtaattcttaacacattgac

SEQ ID NO: 22 - DNA, 459 bp, Homo Sapiens
gcaagtcgcgtgatttctaccacacctgctactgcctgagcggcctgtccatagcccagcacttcggcagcggagccatgttgcatgatgtggtcctgggtgtgcccgaaaacgctctgcagcccactcacccagtgtacaacattggaccagacaaggtgatccaggccactacatactttctacagaagccagtcccaggttttgaggagcttaaggatgagacatcggcagagcctgcaaccgactagaggacctgggtcccggcagctctttgctcacccatctccccagtcagacaaggtttatacgtttcaatacatactgcattctgtgctacacaagccttagcctcagtggagctgtggttctcttggtactttcttgtcaaacaaaaccaatggctctgggtttggagaacacagtggctggttttaaaattctttccacacctgtcaa

SEQ ID NO: 23 - DNA, 549 bp, Homo Sapiens
tgatgtcacctagcagggcttcaggggttcccactaggatgcagagatgacctctcgctgcctcacaagcagtgacacctcgggtcctttccgttgctatggtgaaaattcctggatggaatggatcacatgagggtttcttgttgcttttggagggtgtgggggatattttgttttggtttttctgcaggttccatgaaaacagcccttttccaagcccattgtttctgtcatggtttccatctgtcctgagcaagtcattcctttgttatttagcatttcgaacatctcggccattcaaagcccccatgttctctgcactgtttggccagcataacctctagcatcgattcaaagcagagttttaacctgacggcatggaatgtataaatgagggtgggtccttctgcagatactctaatcactacattgctttttctataaaactacccataagcctttaacctttaaagaaaaatgaaaaaggttagtgtttgggggccgggggaggactgaccgcttcataagccagtacgtctgagctgagta

SEQ ID NO: 24 - DNA, 372 bp, Homo Sapiens
aagcaattttcttgatgcctctgcaagatactgtgaggagaattgacagcaaaagttcaccacctactcttatttactgcccattgattgacttttcttcatattttgcaaagagaaatttcacagcaaaaattcatgttttgtcagctttctcatgttgagatctgttatgtcactgatgaatttaccctcaagtttccttcctctgtaccactctgcttccttggacaatatcagtaatagctttgtaagtgatgtggacgtaattgcctacagtaatgaaaaattaatgtactttaatttttcattttcttttaggatatttagaccacccttgttccacgcaaaccagagtgtgtcagtgtttgtgtg

SEQ ID NO: 25 - DNA, 475 bp, Homo Sapiens
cagggatcggaggacgacccgagtcccaagagtggggttttgctttttaaaaggagagaggaggggtgatggcaggggagtggagggtggccgggcaggtcctgccggcgcagggagccctctgcccttcacactctcctccaaaagagcctccatctgtaaggaagcaggtctccgcgaggggtttctttccatgtgttttcctcctgttgttaaaagaacttttttaaaaaaacagacctcgttttagatttatagcattgacttttacacacattcacacaagaaaaaaatcctttcaaaattcttaaatcttctgttcctcctttttccaagggaagagggcaaaaagtggcctgggctctgttggtgtgcgtgttccgtggcggagagaagaaaatgggaaagacatctcactggtgcttttctcttttgttttagtgccccccgcccccatccctataatatctgtaac

SEQ ID NO: 26 - DNA, 516 bp, Homo Sapiens
gaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctaaataagactggacatttaacttttctaccatttctgcaagttaggtatgtttgccaggagaaaagtatcaagacgtttaactgcagttgactttctccctgttcctttgagtgtcttctaactttattctttgttctttatgtagaattgctgtctatgattgtactttgaatcgcttgacttgttgaaaatatttctctagtgtattatcactgtctgttctgcacaataaacataacagcctctgtgatccc

SEQ ID NO: 27 - DNA, 566 bp, Homo Sapiens
gcgtttccaacctcggagaattccaggcactccccttccccctccgctgacatacttgtataagcggtcatcgttgcgtcatggggcaggcgtggggagcttcctgtcgccttggctgggtgtgggcctggaggaaggtcctggggcgtgcactcgcctgggcagtggggaggagagtggcctgagttacttcacccccgcgtgctgctggttaatgtcccgcgtctctgcaccttcgggtgggagcggggactgatctactttcacattctcaagtttttctcatctgcattagaggtccccagtaggttcccaggttccagcgtgcccctccctcagacacacggacacaatcagccgagaagttcctggtctgaatcacgagaatgtggaggggtggggggtgtcagtggaaaggcataaggctgagctgagaccagttgctggtgaaactgggccaatctggggaggggaacatccttgccagggagtttctgagggtctgctttgtttacctttcgtgcggtggattctttttaactccgtctacctggcgttttgttaga

SEQ ID NO: 28 - DNA, 327 bp, Homo Sapiens
  misc_feature (199)..(199): n is a, c, g, or t
ccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcangggccagcccgccagcccctctgcccacatggtcagccacagtcactccccctctgtggtctcctgaagggagcgcctcctccaacaacacgtggatctgcatggtttgcctgagctttgaacagtca

SEQ ID NO: 29 - DNA, 347 bp, Homo Sapiens
  misc_feature (156)..(156): n is a, c, g, or t
attagtctccaagccattcagtgatgtcttcagcatcactataggactgtctagtgtcactttttacttccttctgggtggaggctttccgactcccaatcatgaaggcaagttaatctttccagttagtgacttttgccccatagttggggtaancacttcctagattgagaaaaagcagctacagtcaatcctgctctgtttgcctcatttggtgatcagtcagtcacacataagttccttgtattctaaatttcatgcacttctcccagatgctatagggttttctctcactgttgccaatggatgtcatccagacagtgggctcatatcttacggttttgtgc

SEQ ID NO: 30 - DNA, 210 bp, Homo Sapiens
agttgatcagagccttccagagtgtggtatgcttttcactgtgtgatgatccttagtggcacatgaatgaacgtccagatgtttgtgcagtagcccacccttatctgcaggatacgttccaagacccccagtgaatgcctgaaactgcagatagtactgaatcctatatatactgtgttttttatgatacatacatgcctatgatgaagt

SEQ ID NO: 31 - DNA, 511 bp, Homo Sapiens
aagagaatgttcctactcacacttcagctgggtcacatccatccctccattcatccttccatccatctttccatccattacctccatccatccttccaacatatatttattgagtacctactgtgtgccaggggctggtgggacagtggtgacatagtctctgccctcatagagttgattgtctagtgaggaagacaagcatttttaaaaaataaatttaaacttacaaactttgtttgtcacaagtggtgtttattgcaataaccgcttggtttgcaacctctttgctcaacagaacatatgttgcaagaccctcccatgggggcacttgagttttggcaaggctgacagagctctgggttgtgcacatttctttgcattccagctgtcactctgtgcctttctacaactgattgcaacagactgttgagttatgataacaccagtgggaattgctggaggaaccagaggcacttccaccttggctgggaagactatggtgctgccttgc

SEQ ID NO: 32 - DNA, 505 bp, Homo Sapiens
aaggcattccacaggatcatcatttaaaaaaaaagaattctggtcctgttttctaaaaaaaaaaaactgttgtagaaattcttaatttggatctatttattagtcagagtttcagctttcttcagctgccagtgtgttactcatctttatcctaaaaatctggaatcagagatttttgtttgttcacatatgattctcttagacacttttatatttgaaaaaattaaaatctttctttggggaaaaattcttggttattctgccataacagattatgtattaacttgtagattcagtggttcaatacctgtttagttgcttgctaatatttccagaaggatttcttgtattggtgaaagacggttggggatggggggatttttttgttcttgttgtacccttgttttgaaactagaaatctgtcctgtggcatgcaaaagaaagcaaattatttttaaaagaaaaaaaccaaagtacttttggtgtcattattccatcttctcca

SEQ ID NO: 33 - DNA, 307 bp, Homo Sapiens
ccagccactgcagatagagacatatggaccacatgttcctgagcttgagatgctaggaagacttgggtatttaaaccatgtcagagctgcctcaccacaggaccttgctggaggctatacttcttctcttgcttgtcacagagcactacaggatgcattcagtgggcttttctggcagcccagttaaccatttataagatttggaccttggagctgaaccagggagctagcaaaagtaaagcagacttataaaattatagctatgtgcagctgcacaacacagtccttccactagcagctgtgttaa

SEQ ID NO: 34 - DNA, 519 bp, Homo Sapiens
  misc_feature (130)..(130): n is a, c, g, or t
caccgcgcagagctcagggggtggtgcgcccggcccttctgcggcgcacagcccagcccaggaacgcgggcggtgcggactcagcgggccgggtgcaggcgcggagctgggcctctgcgcccggcccganctccgtctataaanagagcagccagttgcagggctcnantctgctttccaactgcctgactgcttgttcgtctcactggtgtgagctccagcatcccctttgctcgaaatggaccccaactgctcttgcgccactggntggctcctgcacgtgcgccggctcctgcaagtgcaaagagtgcaaatgcacctcctgcaagaagagctgctgttcctgctnccccgtggnctgtgccaagtgtgcccagggctgcgtctgcaaaggggcatcggagaagtgcagctgctgtgcctgatgtgggaacagctcttctcccagatgtaaatagaacaacctgcacaacctggnatttttttaaaaatacaacactgagccatttgctgcatttc

SEQ ID NO: 35 - DNA, 460 bp, Homo Sapiens
gcagcactcttaacttacgatctcttgacatacggtttctggctgagaggcctggcccgctaaggtgaaaaggggtgtggcaaaggagcctactccaagaatggaggctgtaggaatataacctcccaccctgcaaagggaatctcttgcctgctccatctcataggctaagtcagctgaatcccgatagtactaggtccccttccctccgcatcccgtcagctggaaaaggcctgtggcccagaggcttctccaaagggagggtgacatgctggcttttgtgcccaagctcaccagccctgcgccacctcactgcagtagtgcaccatctcactgcagtagcacgccctcctgggccgtctggcctgtggctaatggaggtgacggcactcccatgtgctgactccccccatccctgccacgctgtggccctgcctggctagtccctgcctgaataaag

SEQ ID NO: 36 - DNA, 540 bp, Homo Sapiens
gctacagattcacactttctggcctaaaccctaatgggatgaggcttttcaccccaggccatgctggtggtgattttttagcccctaaataaaacactggactatttcctgtttacttcattgattgcaactacaaaggtggactcaaagcaaagcacaatcatgccagccaacattccagaattctgctgagaactccaagtctgtgaggggagaggttttacaagccagacaggcctgggggactgcagtccccaaggagaccctgccacatgctggccctttgagtgagaatgctgcatctttctacatatcttcatgagaatactgagaattggattttccttttcaaaatgcactttgctttttttgtatgttttgttatgttgagatgtttctaaagaaaagattttatgtaattataagatgaagcgtagtgaattgtacagctgttgtaataatgacctatttctatataaaataaaattgtatggcttatgtgtaaattattttgtatctgagataccagttccttttccc

SEQ ID NO: 37 - DNA, 367 bp, Homo Sapiens
aaaggggatggacgtctcattctcagggatcctgtctttcattgaggatgtagcccatcggatgctggccacaggcgagtgtactcctgaggatctgtgtttctccctgcaggaaactgtgtttgcaatgctggtagagatcacagagcgagccatggcacattgtggctcccaggaggccctcattgtgggaggagtggggtgtaatgtgaggctacaggagatgatggcaacaatgtgccaggaacgtggagcccggctttttgctacagatgagagattctgtattgacaatggagcgatgatagcccaggctggctgggagatgtttcgggctggacacaggaccccactcagtgattctggg

SEQ ID NO: 38 - DNA, 532 bp, Homo Sapiens
cagaaagtctcagcccaggatggggcttcttcaacagggcccctgccctcctgaagcctcagtccttcaccttgccaggtgccgtttctcttccgtgaaggccactgcccaggtccccagtgcgccccctagtggccatagcctggttaaagttccccagtgcctccttgtgcatagaccttcttctcccacccccttctgcccctgggtccccggccatccagcggggctgccagagaaccccagacctgcccttacagtagtgtagcgccccctccctctttcggctggtgtagaatagccagtagtgtagtgcggtgtgcttttacgtgatggcgggtgggcagcgggcggcgggctccgcgcagccgtctgtccttgatctgcccgcggcggcccgtgttgtgttttgtgctgtgtccacgcgctaaggcgaccccctcccccgtactgacttctcctataagcgcttctcttcgcatagtcacgtagctcccaccccaccctcttcctgtgtctcacgcaagtttta

SEQ ID NO: 39 - DNA, 551 bp, Homo Sapiens
ggatggggcttcttcaacagggcccctgccctcctgaagcctcagtccttcaccttgccaggtgccgtttctcttccgtgaaggccactgcccaggtccccagtgcgccccctagtggccatagcctggttaaagttccccagtgcctccttgtgcatagaccttcttctcccacccccttctgcccctgggtccccggccatccagcggggctgccagagaaccccagacctgcccttacagtagtgtagcgccccctccctctttcggctggtgtagaatagccagtagtgtagtgcggtgtgcttttacgtgatggcgggtgggcagcgggcggcgggctccgcgcagccgtctgtccttgatctgcccgcggcggcccgtgttgtgttttgtgctgtgtccacgcgctaaggcgaccccctcccccgtactgacttctcctataagcgcttctcttcgcatagtcacgtagctcccaccccaccctcttcctgtgtctcacgcaagttttatactctaatatttatatggctttttttcttcgacaa

SEQ ID NO: 40 - DNA, 538 bp, Homo Sapiens
gccagctttgggctgagctaacaggaccaatggattaaactggcatttcagtccaaggaagctcgaagcaggtttaggaccaggtccccttgagaggtcagaggggcctctgtgggtgctgggtactccagaggtgccactggtggaagggtcagcggagccccagtgcctccttgtgcatagaccttcttctcccacccccttctgcccctgggtccccggccatccagcggggctgccagagaaccccagacctgcccttacagtagtgtagcgccccctccctctttcggctggtgtagaatagccagtagtgtagtgcggtgtgcttttacgtgatggcgggtgggcagcgggcggcgggctccgcgcagccgtctgtccttgatctgcccgcggcggcccgtgttgtgttttgtgctgtgtccacgcgctaaggcgaccccctcccccgtactgacttctcctataagcgcttctcttcgcatagtcacgtagctcccaccccaccctcttcctgtgtctcacgcaagtttta

SEQ ID NO: 41 - DNA, 403 bp, Homo Sapiens
tgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaat

SEQ ID NO: 42 - DNA, 437 bp, Homo Sapiens
tggatccaaacctttattatgccattatatgatgccagatgaagaaactccattagcagtgcaggcctgtggactttctcctcgagacattaccactattaaacttctcaatgaaactagagacatgttggaaagcccagattttagtacagttttgaatacctgtttaaaccgaggttttagtagacttctagacaatatggctgagttctttcgacctactgaacaggacctgcaacatggtaactctatgaatagtctttccagtgtcagcctgcctttagctaagataattccaatagtaaacggacagatccattcagtttgcagtgaaacacctagtcattttgttcaggatctgttgacaatggagcaagtgaaagactttgctgctaatgtgtatgaagcttttagtacccctcagcaactggagaaat

SEQ ID NO: 43 - DNA, 520 bp, Homo Sapiens
agccatttgaagacgcctcgtttgcgctgcggacgggggagatgagcgggcccgtgttcacggattccggcatccacatcatcctccgcactgagtgagggtggggagcccaggcctggcctcggggcagggcagggcggctaggccggccagctcccccttgcccgccagccagtggccgaaccccccactccctgccaccgtcacacagtatttattgttcccacaatggctgggagggggcccttccagattgggggccctggggtccccactccctgtccatccccagttggggctgcgaccgccagattctcccttaaggaattgacttcagcaggggtgggaggctcccagacccagggcagtgtggtgggaggggtgttccaaagagaaggcctggtcagcagagccgccccgtgtccccccaggtgctggaggcagactcgagggccgaattgtttctagttaggccacgctcctctgttcagtcgcaaaggtgaacactcatgcggcagcc

SEQ ID NO: 44 - DNA, 530 bp, Homo Sapiens
  misc_feature (68)..(68): n is a, c, g, or t
gattaaacgactgtgtctttgtcacctctgcttaactttaggagtatccattcctgtgattgtagacntttgttgatattcttcctggaagaatatcattcttttcttgaagggttggtttactagaatattcaaaatcaatcatgaaggcagttactattttgagtctaaaggttttctaaaaattaacctcacatcccttctgttagggtctttcagaatatcttttataaacagaagcatttgaagtcattgcttttgctacatgatttgtgtgtgtgaaggacataccacgtttaaatcattaattgaaaaacatcatataagccccaactttgtttggaggaagagacggaggttgaggtttttccttctgtataagcacctactgacaaaatgtagaggccattcaaccgtcaaacaccatttggttatatcgcagaggagacggatgtgtaaattactgcattgctttttttttcagtttgtataacctctaatctccgtttgcatgatacgctttgttagaa

SEQ ID NO: 45 - DNA, 485 bp, Homo Sapiens
tgaatgtacgcttgtccatgctgacctcagtgagtataacatgctgtggcatgctggaaaggtctggttgatcgatgtcagtcagtcagtagaacctacccaccctcacggcctggagttcttgttccgggactgcaggaatgtctcgcagtttttccagaaaggaggagtcaaggaagcccttagtgaacgagaactcttcaatgctgtttcaggcttaaacatcacagcagataatgaagctgattttttagctgagatagaagctttggagaaaatgaatgaagatcacgttcagaagaatggaaggaaagctgcttcatttttgaaagatgatggagacccaccactactatatgatgaatagcactaatacccactgcttcagtgttaacacagcagtgattgtcagctgccaatagcaaatgaagttatgggtgacttgaaataccaaaacctgaggagtgggcaatggtgcttctgtg

SEQ ID NO: 46 - DNA, 351 bp, Homo Sapiens
ttcgccacattggccgtgttggtcttgaactcctggcctcaagcaatccgcctacctcagcctcccaaagtgctaggattacaggcataagccactgagcccagccctagttcagtatcttttatgtaaattataaacatctgcaacattatgtatcatatgcagatacttattgcatttcttttattagtggtgaaagtgttctatgcatttattggctcttgaatttcctcatctatgaattgtcattcacacacctacttttctgcttcgtttttacatatgtctttgcctattaaagatattatccctctgttttatattttctctcattcttgtattgccttttaa

SEQ ID NO: 47 - DNA, 521 bp, Homo Sapiens
ccggaggcaaagagaccgggccgcggaggccaaggaaagggagaacaccgaaaacaataactcctcctccaacaagcagaaccaactctctcctctggaagggggcaagccgctcatgtccagctcagaagaggaattctcacctccccaaagtccagaccagaactcggtccttctgctgcagggcaatatgggccacgccaggagctcaaactattctctcccgggcttaacagcctcgcagcccagtcacggcctgcagacccaccagcatcagctccaagactctctgctcggccccctcacctccagtctggtggacttggggtcctaagtggggagggactggggcctcgaagggattcctggagcagcaaccactgcagcgactagggacacttgtaaatagaaatcaggaacatttttgcagcttgtttctggagttgtttgcgcataaaggaatggtggactttcacaaatatctttttaaaaatcaaaaccaacagcgatctcaagcttaa

SEQ ID NO: 48 - DNA, 498 bp, Homo Sapiens
ggctgagcaccagtgagttctttgcctctactctgaccctagacaacctggggagggaccctgtgcccgcaaaccagacacataggacaaagtttatctataacctggaagaccatgagtggtgtgaaaacatggagtccgttttatagtgactaaaggagggctgaactctgtattagtaatccaagggtcatttttttcttaaaaaaagaaaaaaaggttccaaaaaaaaccaaaactcagtacacacacacaggcacagatgcacacacacgcagacagacacaccgactttgtcctttttctcagcatcagagccagacaggattcagaataaggagagaatgacatcgtgcggcagggtcctggaggccactcgcgcggctgggccacagagtctactttgaaggcacctcatggttttcaggatgctgacagctgcaagcaacaggcactgccaaattcagggaacagtggtggccagcttggaggatggac

SEQ ID NO: 49 - DNA, 331 bp, Homo Sapiens
gagacgtggtaagtgcggtgcagttttcaactgacctctggacgcagaacttcagccatgaaggtaacaggcatctttcttctcagtgccttggccctgttgagtctatctggtaacactggagctgactccctgggaagagaggccaaatgttacaatgaacttaatggatgcaccaagatatatgaccctgtctgtgggactgatggaaatacttatcccaatgaatgcgtgttatgttttgaaggtcggaaacgccagacttctatcctcattcaaaaatctgggccttgctgagaaccaaggttttgaaatcccatcaggtcaccgc

SEQ ID NO: 50 - DNA, 548 bp, Homo Sapiens
agccatcccatgttagagcttctcaagaggaagacagcccagactctttcagttctctggattctgagatgtgcaaagactaccgagtattgcccaggataggctatctttgtccaaaggatttaaagcctgtctgtggtgacgatggccaaacctacaacaatccttgcatgctctgtcatgaaaacctgatacgccaaacaaatacacacatccgcagtacagggaagtgtgaggagagcagcaccccaggaaccaccgcagccagcatgcccccgtctgacgaatgacaggaagattgttgaaagccatgagggaaaaaataaaccccagttctgaatcacctaccttcaccatctgtatatacaaagaattcttcggagcttgtcttatttgctatagaaaacaatacagagcttttgggaatggaatcactgattttcagtcttttccatttctttcctcctagaatctgtgatctgagggtataaagacatttccaccaagtttgagccctcaaaatgtcctgattacaatgctgtctgtcc

SEQ ID NO: 51 - DNA, 526 bp, Homo Sapiens
gtccacattcctgcaagcattgattgagacatttgcacaatctaaaatgtaagcaaagtagtcattaaaaatacaccctctacttgggctttatactgcatacaaatttactcatgagccttcctttgaggaaggatgtggatctccaaataaagatttagtgtttattttgagctctgcatcttaacaagatgatctgaacacctctcctttgtatcaataaatagccctgttattctgaagtgagaggaccaagtatagtaaaatgctgacatctaaaactaaataaatagaaaacaccaggccagaactatagtcatactcacacaaagggagaaatttaaactcgaaccaagcaaaaggcttcacggaaatagcatggaaaaacaatgcttccagtggccacttcctaaggaggaacaaccccgtctgatctcagaattggcaccacgtgagcttgctaagtgataatatctgtttctactacggatttaggcaacaggacctgtacattgtcacattgcat

SEQ ID NO: 52 - DNA, 476 bp, Homo Sapiens
tgggggacttatttgttggggatcttaaataagattccttttgatctaccggaatatacatgtacagagtacattggatcatgttggaaagaaggcaagtgaaaaggtcagagatgaagtagcgaagttatggaatatcgtggaaaggatactagttgtgaaatggaaagagacaagttatagtaccccaaaagcaaaacaagcaggagatgcaagagatgccccaaaaggacaaagcaacaattttctgttgccacctttataccggaagactctgttgtagaagaaaagaaggctttggtgcaccttatgtgggaggaggaggggcagggcatgctgatgctgagcgtacaggcagacaagagcgtagcctgctgttgcctccatcactatgaaatgacttattttacctgaaggacccatggtttatgttcctctaattcctttcactctccctaagccctctgagagagatg

SEQ ID NO: 53 - DNA, 501 bp, Homo Sapiens
gcctgtcggctcagatcgaggaatgcatcttccgggacgttggaaacacagacatgaagtataagaaccgtgtacggagtcgtatctccaacctgaaggatgccaagaaccctgacctgcggcggaatgtgctgtgtggggccataacaccccagcagatcgctgtgatgacctcagaggagatggccagtgatgagctgaaggagatccgtaaggccatgaccaaggaggccatccgagagcaccagatggcccgcactggcggcacgcagacagacctgttcacctgcggcaagtgcaggaaaaagaactgcacctacacacaggtgcagacccgcagctctgatgagcccatgaccacctttgttgtctgcaacgagtgtggaaaccgctggaagttctgctgacccctcgtgtagatgtgctgcagccttgggccctccccggcccacgtcctccgttgacacagcttctctggagaccctagaaggcggcatgtcc

SEQ ID NO: 54 - DNA, 453 bp, Homo Sapiens
tggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagt

SEQ ID NO: 55 - DNA, 498 bp, Homo Sapiens
  misc_feature (142)..(142): n is a, c, g, or t
acccttggccatcaggcgaggggctgggcctgtgcagctgggcccttggccagagtccactcccttcctggctgtgtcaccccgagcagctcatccaccatggaggtcattggcctgaggcaagttccccggagagtcgggntcccctgtggccccctcaggcctatgtctgtgaggaaggggccctgccactctccccaagagggcctccatgtttcgaggtgcctcaacatggagccttgcctggcctgggctaggggcactgtctgaactcctgactgtcaggataaactccgtgggggtacaggagcccagacaaagcccaggcctgtcaagagacgcagagggcccctgccagggttggccccagggaccctgggacgaggctgcagaagctctccctccctactccctgggagccacgtgctggccatgtggccagggacggcatgagcaggaggcggggacgtgggggccttctggtttggtgtcaacagc

SEQ ID NO: 56 - DNA, 544 bp, Homo Sapiens
gaagccacaaagatgccacatgttagtatatcagtgagaggtgactccacagtgctctctggagaagcaatatgagtgactgaagagtggggccttttgcttttgcctggatataggggtgctcttctactgtaattgggtgtggaaaaactctggctttatggtattccattaggttcttttcatttaaagtagtcttaaaatcaaagtatccaatattttaaagccacaaagtagattacataattagcagagattttagtcagtaaaatgttagaaatcaaactataagaaaattcaagtcctttattttgtgtcttgggtatatgtcattattttaaattccacactcccttatttaatcactttggtaagtgcctttgatgttttgaaatgtatagtgggagatgagcaaatgtaaatgtcatgtgccctgttccctagcttctcaattcctcataaccatttttaccagtgttgcaaagtttagacctttgtgttaatatcagaagtgtatttgtagcccctccatagtgaacaatga

SEQ ID NO: 57 - DNA, 535 bp, Homo Sapiens
gccgaagaagcctgtctgtggggtgtgtcgcagcgctctggcacctggcgtccgagccgtggagctcgagcggcagatcgagagcacagagacttcttgccatggctgccgtaagaatttcttcctgtccaagatccggtcccacgtggctacttgttccaaataccagaattacatcatggaaggtgtgaaggccaccattaaggatgcatctcttcagccaaggaatgtcccaaaccgttacacctttccttgtccttactgtcctgagaagaactttgatcaggaaggacttgtggaacactgcaaattattccatagcacggataccaaatctgtggtttgtccgatatgtgcctcgatgccctggggagaccccaactaccgcagcgccaacttcagagagcacatccagcgccggcaccggttttcttatgacacttttgtggattatgatgttgatgaagaggacatgatgaatcaggtgttgcagcgctccatcatcgaccagtgagcagagtccgtgcttgctatc

SEQ ID NO: 58 - DNA, 479 bp, Homo Sapiens
gctgaaagaagcccacatagaactgcttagggacagcaccactgactccaaagaaaatcccagcagaaagagaaatggaatgtgcacggatacacattcactgctcagtaagaggctcaagacatgactgatttgcattttaaagcaagatgcgatgtccagagttacagagaatgagtagatgtgtctcatcggttaatagctctattatacctctaaaggtggaattgtcagtttagattcataaatgaaaaggtaaatgagtaatcagaataaaccaagtgataatcaaaccatgtcaagattattagttcagactctagcctgttaattttcttagttgatttctgaagctacctgatttattctattaaattgtaagcttgcaaactcaaaataaattggcagatttacctctcatgttttaatgtgtcaaattagagagcaaagtataacaggtgccttcacttttgagactt

SEQ ID NO: 59 - DNA, 518 bp, Homo Sapiens
gtgccatagtgcaggcttggggagctttaagcctcagttatataacccacgaaaaacagagcctcctagatgtaacattcctgatcaaggtacaattctttaaaattcactaatgattgaggtccatatttagtggtactctgaaattggtcactttcctattacacggagtgtgctaaaactaaaaagcattttgaaacatacagaatgttctattgtcattgggaaatttttctttctaacccagtggaggttagaaagaagttatattctggtagcaaattaactttacatcctttttcctacttgttatggttgtttggaccgataagtgtgcttaatcctgaggcaaagtagtgaatatgttttatatgttatgaagaaaagaattgttgtaagtttttgattctactcttatatgctggactgcattcacacatggcatgaaataagtcaggttctttacaaatggtattttgatagatactggattgtgtttgtgccatatttgtgccatt

SEQ ID NO: 60 - DNA, 489 bp, Homo Sapiens
gggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcatggagcttaggaaacgctcccactcccacgggccatcgagggccagcacggctgagcggtgaaaaaccgtagccatagatcctgtcc

SEQ ID NO: 61 - DNA, 472 bp, Homo Sapiens
atttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtata

SEQ ID NO: 62 - DNA, 523 bp, Homo Sapiens
  misc_feature (41)..(41): n is a, c, g, or t
gacaacagccctggaggggaacagagtgagagagatgtttngctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatcctgaatggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttggtagtagtaacttaccttaccttaccnanaaaaatattaatgtaagccatataacatgggattttcctcaannannnnannnnnnccttttgtacttcactcagatacta

SEQ ID NO: 63 - DNA, 373 bp, Homo Sapiens
gggcagatcttggactcatgaggaggggcccccctgcccagaggggtcaacccttctggaaactgtgaagatctgacttcgcccccccccccccccatcttcgggaccaggatttgcacagaagcacatgcacctacccatacaccccctcttctgagcgtccctgttcccccatctcgctccctcccaggactctgaccccagcattctcaggcaccagtccctgtccggaatgccacccacatcttccatttccatgtcccctcccagagctggtggacccagggaacagccactcccctccactctctaccagataactgaggaggggagaggtgggccgtaacgggcacggatcacgatgtaaattatt

SEQ ID NO: 64 - DNA, 535 bp, Homo Sapiens
agcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggcagtcctgggtgtggggacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaa

SEQ ID NO: 65 - DNA, 452 bp, Homo Sapiens
catgctggaccagatcaactcctgtctggaccacctggaggagaagaatgaccacctccacgcccgcctccaggagctgctggagtccaaccggcagacacgcctggagttccagcagcagctcggggaggcccccagtgatgccagcccctaggctccaagagcccccaaccgggacccaaccctgcctccctgggctaggctctggcctgggcactcaccccctggcttagacaccttctcaagggctggccttcagggacccctggtgggtctgcctgcctgggccacccttcctgcctgggcctccccttggcctacctgggccagcccccaccacctggcatgccctcctggggccaagagtgggcctgcaacccacccacttgcctgcccacccaactcctgggcgctccccactctgcccaggccttgagtgtccacattaaatg

SEQ ID NO: 66 - DNA, 323 bp, Homo Sapiens
cacttaccagtgagcatatatattttaaaatactttctttggatattgtaattcttaactggttgtaaattagaaaagctgggattacatatggtgtgcggttacagtctaaattttttcatcctcctatgcatcataagcatgtttgtaatattttcaaaaatagttctactgatgctacaggaatttcaagcctgtggtgaatgttagtatttaccatagggagtgaagtggagttatggtttcattcaatagagtattgctgattatacttgagtggaatcctttcctcacgtactcccacagacgtctgggcctggaaa

SEQ ID NO: 67 - DNA, 560 bp, Homo Sapiens
ggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaa

SEQ ID NO: 68 - DNA, 471 bp, Homo Sapiens
gttttgggtatgtttaatctgttatgtactagtgttctgtttgttattgttttgttaattacaccataatgctaatttaaagagactccaaatctcaatgaagccagctcacagtgctgtgtgccccggtcatctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcg

SEQ ID NO: 69 - DNA, 518 bp, Homo Sapiens
aattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatccgttcaagaagctggctgacatgtacggaggcg

SEQ ID NO: 70 - DNA, 182 bp, Homo Sapiens
cttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggca

SEQ ID NO: 71 - DNA, 538 bp, Homo Sapiens
tgaggagccagcgtctagggcagcagccgcttcctagaagaccaggtcatgatgatgggcagcgcccgagtggcggagctgctgctgctccacggcgcggagcccaactgcgccgaccccgccactctcacctgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccacggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttccccc

SEQ ID NO: 72 - DNA, 513 bp, Homo Sapiens
atattagttaccctggtgtgctgtattctctaaaacctttaaatgtttgcatgcagccattcgtcaaatgtcaaatattctctctttggctggaatgacaaaaactcaaataaatgtatgattaggaggacatcataacctatgaatgatggaagtccaaaatgatggtaactgacagtagtgttaatgccttatgtttagtcaaactctcatttaggtgacagcctggtgactccagaatggagccagtcatgctaaatgccatatactcacactgaaacatgaggaagcaggtagatcccagaacagacaaaactttcctaaaaacatgagagtccaggctgtctgagtcagcacagtaagaaagtcctttctgctttaactcttagaaaaaagtaatatgaagtattctgaaattaaccaatcagtttatttaaatcaatttatttatattcttctgttcctggattcccattttacaaaacccactgttctactgttgtattgcccagt

SEQ ID NO: 73 - DNA, 530 bp, Homo Sapiens
ggatttgtgttcttacagtacttgaaaatatttaaggaagagatgaagctctgcagttttttctatgtgggatgattacttttttaaggaggattaattctgaggtagtatagtaactaaaggggaatatatgaattgtttaacaaattagaatttgtttacaactacttgaatttttaaattatgtcaaaacttacattacttgccaagcagtatgatgttataggaaacataaataagattacagaggtatcaatttggttaaaattcaccattttataagactaagcaataatcttaacaacctctttcctgaatatttaaatgtgtttgtatggtgttatgactaattgttactgatttagagactaagccctcttaaaacctttagttaaatataaaaagaaattatatatatcttgcctccctgatggaaaactatataaaattgtagacttaaaaggtttgtggaaatacattaggatatcagaaaactaaatatatggagttgctttatgactattacatgt

SEQ ID NO: 74 - DNA, 406 bp, Homo Sapiens
ggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatataacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaacccta

SEQ ID NO: 75 - DNA, 286 bp, Homo Sapiens
agcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgc

SEQ ID NO: 76 - DNA, 436 bp, Homo Sapiens
gaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccact

SEQ ID NO: 77 - DNA, 429 bp, Homo Sapiens
tcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgc

SEQ ID NO: 78 - DNA, 195 bp, Homo Sapiens
tccccctgtagactagtgccgtgggagtacctgctgcccagctgctgtggccccctccgtgatccatccatctccagggagcaagacagagacgcaggatggaaagcggagttcctaacaggatgaaagttcccccatcagttcccccagtacctccaagcaagtagctttccacatttgtcacagaaatcagag

SEQ ID NO: 79 - DNA, 301 bp, Homo Sapiens
tggtgttgggagccctttggagaacgccagtctccaggtccccctgcatctatcgagtttgcaatgtcacaacctctctgatcttgtgctcagcatgattctttaatagaagttttatttttcgtgcactctgctaatcatgtgggtgagccagtggaacagcgggagcctgtgctggtttgcagattgcctcctaatgacgcggctcaaaaggaaaccaagtggtcaggagttgtttctgacccactgatctctactaccacaaggaaaatagtttaggagaaaccagcttttactgttt

SEQ ID NO: 80 - DNA, 459 bp, Homo Sapiens
  misc_feature (164)..(164): n is a, c, g, or t
ggaaacgttcccagttcattttcagtcctgttgtgagcacagttctgaagggtttattattgtcaaaataagttttgttttgttttgtttatgttgggtttttaatgttgtctcttgacccttaatgctcaggttcttgtgggagttaatcagccacatccaangttaccttgagggggaagaagagggtgatgctcagaagctaaacaagacaggggccacatgaccctctattgattagccccaagtagaaagtcctgtggttttatgtttaatggtaatagttgatcatatatggcataattttctatcagcttcctactcagtcactataaacacagacttgaaatagtactttaaatgtccaaatacctaaatgtgctaaactggaggtaactatttctaggtagttgaatttttgaaagtcatgatcagccacacaactgttttgtacatact

SEQ ID NO: 81 - DNA, 394 bp, Homo Sapiens
aatccttattgttcagagttgtttgggggttctgtttcagagcataaaacctaaaggttatagtagaacaaggcaccttcttaaaagaaatcttgcttcagaccatcagttacagagaatttcctaaagtaaaattgaagcaactacaacttctccttagacactttggaatctaaccacttaaggacctttttaaagagatagcttctcttctttctgaagatcaatttctcccaaggccaagattgtccttttctcccatttcttgctagctattgcaaatgagggaagaacattattcatctctcctccccttttttttctgattcttttttcagtcagttttgctcctgggttcaagtagtattaccaccctttcacaagcaacagactc

SEQ ID NO: 82 - DNA, 514 bp, Homo Sapiens
  misc_feature (89)..(89): n is a, c, g, or t
gctcactacactattcattgcacacaaatgaatttttcactttttaagatgcattcttggtgctcaaaccagatcgaagtttgtctctnaaagctattgtctgcacaggctgctgcatgctctgttgttaaatggatggacaggctattctaaattttggttgatacttttgctactatgggcaattaacttgaaaaaaataatcgatcccaactctgtgctctgatgtacctcttctgccccttttatgacacctttgaccaaatgccttctatggttcacagtgcaggcacaaaactacctctgatacagaaggttctttacaagcttattttacataccgtgaatccctcacctaaagggagaggtgaaagcaaagactgctttgaatgggtattgagggagattgtgtccataccaagccaccctgaagaagtatttcacttgcagtagaactgtggatttgtgctgtcatttcaccttggaataaacacctatctctaagcaggaccaa

SEQ ID NO: 83 - DNA, 299 bp, Homo Sapiens
caccaaattacctaggctgaggttagagagattggccagcaaaaactgtgggaagatgaactttgtcattatgatttcattatcacatgattatagaaggctgtcttagtgcaaaaaacatacttacatttcagacatatccaaagggaatactcacattttgttaagaagttgaactatgactggagtaaaccatgtattcccttatcttttactttttttctgtgacatttatgtctcatgtaatttgcattactctggtggattgttctagtactgtattgggcttcttcgttaat

SEQ ID NO: 84 - DNA, 219 bp, Homo Sapiens
ttatcgccctgagaagatctaccccagggagaatctgagacatcttgcctacttttctttattagctttctcctcatccatttcttttatacctttcctttttggggagttgttatgccatgatttttggtatttatgtaaaaggattattactaattctatttctctatgtttattctagttaaggaaatgttgagggcaagccaccaaattacctag

SEQ ID NO: 85 - DNA, 518 bp, Homo Sapiens
  misc_feature (61)..(65): n is a, c, g, or t
aaggactggtatctttctgtgagcaataaggactggataaagactgcatatccttgtgtcnnnnncagcancnatacaataaggagggttttaatgtgaagcaggcaatctnccagccccttctggtcttggatgaaatagttgcacagagtattgcaccaanaatacacaatggaggctgaaaagttcaacatattttaagtcaattaatcaaattgcattgattcttgatgctttcttagaggcctacatgatttcttagattgctctgataaactatcataaggggtccacntcccctcatttagctcccccagggatttcttttcccccatgtcatacacccagtcctaaatcaacccccaaggctatccttccatcccttctgcagagggaacttttgtcagactctgcaacaaactcctagctctatccagagtgtcctctgctgctaagattggtatctttctcctcaaaagcctggatggtgaatgggggtgcattagtcagaattctcc

SEQ ID NO: 86 - DNA, 458 bp, Homo Sapiens
taaaaacctgtatctgacccactttgtaatttttgctccaatatccattctgtagacttttgaaaaaaaagtttttaatttgatgcccaatatattctgaccgttaaaaaattcttgttcatatgggagaagggggagtaatgacttgtacaaacagtatttctggtgtatattttaatgtttttaaaaagagtaatttcatttaaatatctgttattcaaatttgatgatgttaaatgtaatataatgtattttctttttattttgcactctgtaattgcactttttaagtttgaagagccattttggtaaacggtttttattaaagatgctatggaacataaagttgtattgcatgcaatttaaagtaacttatttgactatgaatattatcggattactgaattgtatcaatttgtttgtgttcaatatcagctttgataattgtgtaccttaag

SEQ ID NO: 87 - DNA, 336 bp, Homo Sapiens
gggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttg

SEQ ID NO: 88 - DNA, 521 bp, Homo Sapiens
atatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctat

SEQ ID NO: 89 - DNA, 503 bp, Homo Sapiens
gtggctatccactgttagttcagaagctgggcttggactactcttatgatttagctccacgagccaaaattttccggcgtgaccaagggaaagtgactgatacggcatccatgaaatatatcatgcgatacaacaattataagaaggatccttacagtagaggtgacccctgtaataccatctgctgccgtgaggacctgaactcacctaacccaagtcctggaggttgttatgacacaaaggtggcagatatctacctagcatctcagtacacatcctatgccataagtggtcccacagtacaaggtggcctccctgtttttcgctgggaccgtttcaacaaaactctacatcagggcatgccagaggtctacaactttgattttattaccatgaaaccaattttgaaacttgatataaaatgaaggagggagatgacggactagaagactgtaaataagataccaaaggcactattttagctatgtttttcccatcagaat

SEQ ID NO: 90 - DNA, 275 bp, Homo Sapiens
ccccatcacggagggtccagactgtccactcgggggtggagtgagactgactgcaagccccaccctccttgagactggagctgagcgtctgcatacgagagacttggttgaaacttggttggtccttgtctgcaccctcgacaagaccacactttgggacttgggagctggggctgaagttgctctgtacccatgaactcccagtttgcgaattaataagagacaatctattttgttacttgcacttgttattcgaaccactgagagcgagatgg

SEQ ID NO: 91 - DNA, 405 bp, Homo Sapiens
tcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgt

SEQ ID NO: 92 - DNA, 375 bp, Homo Sapiens
aagctatgtgtatcttctgtgtaaagcagtggcttcactggaaaaatggtgtggctagcatttccctttgagtcatgatgacagatggtgtgaaaaccatctaagtttgcttttgaccatcacctcccagtagcaatttgctttcataatccatttagcaatccaggcctctgttgaaaagataatatgagggagaagggaacacatttccttctgaacttacttccctaagtcactttccttatgtatcatctaatacaatgatggttgagtgaaaatacagaaggggtgtttgagtattcagatttcataaaacacttccttggaatatagctgcattaacttggaaagaagcctgttgggccagaagacaga

SEQ ID NO: 93 - DNA, 533 bp, Homo Sapiens
gctggtgtgtgtgtcaaaccctcactcacccacgcactcacacacagcattctgttctccatgcaaagttaagatcgaatccatccgcttgtaggggaaaaaaaggaaaaaaattaaccagagagggtctgtaatctcgcagagcacaggcagaatcgttccttccttgctgcatttcctccttagactaatagacgttttggaaagttcggctagtgttcgtgtgtttgtcgtagcacccagagcctccaccaaaccctctccatgtctttacctcccagtcgctctaagatctgcttgaagtctcgtatttgtactgctttctgcttttctcccacccctcctagcacccccacatcccccatctagtaacatctcagaaatttcatccagaggaacaaaaaaattaaaaatagaacatagcaaagcaaagacagaatgcccccccccaaatattgtcctgtccctgtctgggagttgtgttatttaaagatattctgtatgttgtatcttttgcatgtagcttccttaat

SEQ ID NO: 94 - DNA, 413 bp, Homo Sapiens
atctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagcacttggtgaagacaaggccgtcaggacccaccatgtctgccccatcacgcggccgagacatggcttggccacagctcttgaggatgtcaccaattaaccagaaatccagttattttccgccctcaaaatgacagccatggccggccggtgcttctgggggctcgtcggggggacagctccactctgactggcacagtctttgcatggagacttgaggagggcttgaggttggtgaggttaggtgcgtgtttcctgtgcaagt

SEQ ID NO: 95 - DNA, 465 bp, Homo Sapiens
cagcccggccagttggagttgtagtaccacgagggacgccaactcccagaggagtcctgtccacccgagggccagtgagtcggggaagaggacttctcactcccagagcaagaggagtccccccaactgggtacagacctccaccgccacccccgacacaagagacttatggagaatatgactatgatgatggatatggcactgcttatgatgaacagagttatgattcctatgataacagctatagcaccccagcccaaagtggtgctgattactatgattacggacatggactcagtgaggagacttatgattcctacgggcaagaagagtggactaactcaagacacaaggcaccttcagcgaggacagcaaagggcgtctacagagaccagccatatggcagatactgattgtactgtctgatgttgtgaaatagccaatctccaccagtcctgtatactg

SEQ ID NO: 96 - DNA, 537 bp, Homo Sapiens
gagaacacggtggcagagacggagtgccgctatgccctgcagctgcagcagatccagggactcatcagcagcatcgaggcccagctgagcgagctccgcagtgagatggagtgccagaaccaagagtacaagatgctgctggacatcaagacacgtctggagcaggagatcgccacctaccgcagcctgctcgagggccaggacgccaagaagcgtcagcccccgtagcacctctgttaccacgacttctagtgcctctgttaccaccacctctaatgcctctggtcgccgcacttctgatgtccgtaggccttaaatctgcctggcgtcccctccctctgtcttcagcacccagaggaggagagagccggcagttccctgcaggagagaggaggggctgctggacccaaggctcagtccctctgctctcaggaccccctgtcctgactctctcctgatggtgggccctctgtgctcttctcttccggtcggatctctctcctctctgacctggatacgctttggtttctcaacttc

SEQ ID NO: 97 - DNA, 372 bp, Homo Sapiens
aactttaacttagagcttcattactttaagaatggaaaacaacctctgagtttgatttcccaaagtttcataaagcccctaagctcatgattttcatcaactctttgcccacatagtcatttacctccacagccgtttgttgtcatagaaggggtggtggtgtttggatttgatttttttcaacttgcagtgagaaataggataggtgacaaaaccttacttgttttcttaagacaattcagtgcttgagcatctctgtcagaaatggaatgaaatactgttagccaattagaattattttatgtattgttattgtgttttgctgatttttatatgaaaatataattattcattcttgatctctggaagcaa

SEQ ID NO: 98 - DNA, 365 bp, Homo Sapiens
gggagccaaggctttatacgtctaaagaaaatattcagtagctgaatccgcccagtgatagcctgtgggcaccagcagcaagggctgccatgggatacagcacccatctacaaagacctctattacataaacactgcttcttacaggaaacaaacctcttctgggatctccttttgtgaaaaccagtttgatgtgctaaaagtaaaaagtctattttccagtgtggtcttgttcagaagcagccagatttccaatgttgtttttcccctccactcagaaacccctgccctttcccttcagaaaacgatggcaggcattcctctgagtttacaagcagagactcactccaacccaaactagctggg

SEQ ID NO: 99 - DNA, 465 bp, Homo Sapiens
  misc_feature (110)..(110): n is a, c, g, or t
acacacacatgcaattttgcttaacaaaagtattttataatacagtttcatacagaattaccttaaaagggagtcttatgttttcaactacagatagttgtaagggatcntacagaagatattgatgatagttgaaatattcttagaaggggtgtgtatgtctagctgtgtctaccatgtgtatgtattcttgacaagcagtataaaatacctgtgatttttctttacattagggataatgcataaggaattaatcttcatatatattatcatccctaatgtagcagggggaagtatttaattgcccatgatatgtattttacttatactatgccagagaggaaactataaagtaattacacatgtaatcttgggtttttcacatatgtaggtattcattttgagtaggttgaagaagaaaaaaaatatttaaatgaattgaattcctgatgggatagtatcaat

SEQ ID NO: 100 - DNA, 515 bp, Homo Sapiens
gaactctgcatcttcatggtttacagaaattggtgcaggcagccagcagttagattccattcatgtaacacagttggagagagataccgttttagtgtgtttagacaaatttgtgaaaattgtaaatctacaaggaaaattaaaatcaagtaagaaactggcctctgagttaagttttgattttcgcattgaatctgtagtatgccttcaagacagtgtgttggctttctggaaacatgggatgcagggtaaaagcttcaagtcagatgaggttacccaggagatttcagatgaaacaagagttttccgcttattaggatcagacagggttgtcgttttggaaagtaggccaacagaaaatcctactgcacacagcaatctctacatcttggctggacatgaaaatagttactaagcaacagaaactgatctcaaatgacaggaaaatgaatatactccattgaaagggaaaataaggaaattcaatacaaactgcactatgatttgctttaact

SEQ ID NO: 101 - DNA, 525 bp, Homo Sapiens
ctcagagccacccctaaagagatcctttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaat

SEQ ID NO: 102 - DNA, 418 bp, Homo Sapiens
gcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtc

SEQ ID NO: 103 - DNA, 462 bp, Homo Sapiens
aacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaaca

SEQ ID NO: 104 - DNA, 370 bp, Homo Sapiens
  misc_feature (168)..(168): n is a, c, g, or t
gcaaatatcttaccaggcagcctatgaattaacccaaagaagctttggttggttttggtggatttttatcatgccatgttggacatgagattttttagatcttccttcccacattgctagacgtctcactcaaagacatttgttgggagtcacatttgcatcatagangagacagtccattcatcttagttaaattggattgagaatgccttttgtttccaggaaaatattgatcaccatgaaagaagaatagttttttgtccccagagacattcatttagttgatataatcctaccagaaggaaagcactaagaaacactcgtttgttgtttttaaaggcaacagacttaaagttgtcctcagccaagg

SEQ ID NO: 105 - DNA, 434 bp, Homo Sapiens
caggtgtatctgcacagtggtcgccccacagcagaccatgtgttcacgggatgcccgcacaaaacagctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaagggctaacttaaaagagttttttcaatgctgcagtgactgaagaagcagtccactcccatgtaaccatgaaagagagccagagagctttttgcaccatgcatttttactattattttccaatacttagcaccatttcactaaggaaccttgaatacaaccaggatcctcctttgcatgcgactgtagctgc

SEQ ID NO: 106 - DNA, 503 bp, Homo Sapiens
  misc_feature (158)..(158): n is a, c, g, or t
gcgggccacagacgtcggaagaaactcccgtatttgcagctggaactgcagcccacggcgccccggttttcctccccgccctgtccctctctggtcaaacaacatactaaagaggcgaggcaatgactgttggccagttctcaccggggaaaaacccnactgttaggatggcatgaacatttccttagatcgtggtcagctccgaggaatgtggcnnccaggctctttgangagccatgggctgcacccnggccgtaggcntagtgtaactcgcatcccattgcagtgccngtttcnttgactgtgttgctgtctcttagattaaccgtgctgaggctccnacatagctccntggacctgtgtcntagtacatactgaagcgatggtcagagtgtgtagagtgaagttgctgtgcccacattgtttgaactcgcgtaccccgtagatacattgtgcaacgttcttctgttattcccttgaggtggtaacttcgtatgttcagt

SEQ ID NO: 107 - DNA, 556 bp, Homo Sapiens
ggagacttgagcttgacctaaggatatgcattaaccactctacagactcccactcagtactgtacagggtggctgtggtcctagaagttcagtttttactgaggaaatatttccattaacagcaattattatattgaaggctttaataaaggccacaggagacattactatagcatagattgtcaaatgtaaatttactgagcgtgttttataaaaaactcacaggtgtttgaggccaaaacagattttagacttaccttgaacggataagaatctatagttcactgacacagtaaaattaactctgtgggtgggggcggggggcatagctctaatctaatatataaaatgtgtgatgaatcaacaagatttccacaattcttctgtcaagcttactacagtgaaagaatgggattggcaagtaacttctgacttactgtcagttgtacttctgctccatagacatcagtattctgccatcatttttgatgactacctcagaacataaaaaggaacgtatatcacataattccagtcacagtttttggttcctctt

SEQ ID NO: 108 - DNA, 543 bp, Homo Sapiens
ctgacctctttgaagttgcagaatgctttgaaattctaatggtatctgaaatatcagctcatagaaagtaacaaaatttgctgtcaccttaaataagacattttaattttgttataatgtacaatttagaagtttgattaattatattatctatttaggcattaatataaaagaggtaggagtctgttatttaaaaaaagcattaaatttaaaaaaaaactgtcttgtctacttttagcttcattctcccatattttgaagggtgtgtaacttcagctctgcaggattgcatggggtaaaacttgttaccaacacatgtgaaccattgctacattgtaggttgtgatcattttgccccactgaagcccatgtatctgaccttacgtgccttttgaactaggagaatcgggctaatttattaatgatgataattataatgtatctgtacagcactttttacatttgcgaagtgctttccaatccatgttagttactagttattacagctgtaaggataaaacacgtcatgtggattcattttga

SEQ ID NO: 109 - DNA, 458 bp, Homo Sapiens
agaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtt

SEQ ID NO: 110 - DNA, 412 bp, Homo Sapiens
gtcaaaccatgactcgcacatggcaaaagaacgggcccacagtacagcctcacattcttcttccaattctgaagatacagagatgtgatgaaaacaagtaatagctttggctgtttatttgatagctgtttctgggtatttaataggaatcctttctcaaggaatgagttgtgacctgtttactgtctctttagaagaaaaactccactggaaaccattcaccatgtgtgactgtcttctgttatcatttgtcttacaggcggctattgcagacggctaatttatgcttaacttaggaagagataaggcaagagctagatttttttcatgtgatcttttccaagcttcaacttaacttaactacatttctctgtatgatgatgtctcttacttctacaggttccttgagcac

SEQ ID NO: 111 - DNA, 514 bp, Homo Sapiens
taaattcacatgcagtctcagagactatttagacaaagttcaagttaggagcttttaggatgtgggagtaaaactttaatgggaggggagggctggctgctggaagaaggaagaagccagactggttagacagtactcttaactcctagcccagcctacgtgccctgcccctctggccactgctgcagacacctgccttaacacacacacctctaggactccacagttttgccttaaaggaccttcccaagtctccctttccctgtctggcttctcccttaagaagagagagatacttgtagaattgggtggggggaatgagcatgaactgtccttccatttgggatatgttacattagagtgagagagagaataaggagcctttcttatggaagaaatgggagaagagagacagggttcttttcagcagagtctagtagtttctctgtaaggcaaaataatctaaaaagactaacctgcccacccactccttatattgctgtgagattgcccc

SEQ ID NO: 112 - DNA, 489 bp, Homo Sapiens
cggacccatccaagtcatctgattgaagagcatgacagaaacaaaatgtattcaccaagcattttaggatttgactttttcactaaccagttgacgagcagtgcatttacaaggcactgccaaacaagatgcccttgggagctgtgagggaaagaggacctgcgggcttagatcaatctcaattccttttcatgccctcctgcattgctgctgcgtgggtatttgtctccttagccatcaggtacagtttacactacaatgtaagctataggtggagcatcagcagtgagtgaggccattcttcatccttaggatgtggcaatgaaatgatggtgcaagttcctttctcttttgtgaatctttccccccatttcctgtttacatgtaacccaacaaaatgcaatttctagtgccttctgtccaatcagttctttcctctgagtgagacgtacttggctacagatttctgccttgttttgcgacattgtc

SEQ ID NO: 113 - DNA, 416 bp, Homo Sapiens
gattggtatggccttagctcttagccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgaagggagccggtccttgct

SEQ ID NO: 114 - DNA, 502 bp, Homo Sapiens
cccgaccggtgggcatttgtgaggcccatggttgagaaatgaataatttcccaattaggaagtgtaagcagctgaggtctcttgagggagcttagccaatgtgggagcagcggtttggggagcagagacactaacgacttcagggcagggctctgatattccatgaatgtatcaggaaatatatatgtgtgtgtatgtttgcacacttgttgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtacttattctgcagcatgacctgtgaccagcactgtctcagtttcactttcacatagatgtccctttcttggccagttatcccttccttttagcctagttcatccaatcctcactgggtgggg

SEQ ID NO: 115 - DNA, 430 bp, Homo Sapiens
accacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaagatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacactt

SEQ ID NO: 116 - DNA, 449 bp, Homo Sapiens
gggttgccatccaagtgaaagtcttttccttgaccaagggggacagtcagttttgcaaaaggactctaatacctgtttaatattgtcttcctaattgggataatttaattaacaagattgactagaagtgaaactgcaacactaacttccccgtgctgtggtgtgacctgagttggtgacacaggccacagaccccagagcttggcttttgaaacacaactcagggcttttgtgaaggttcccccgctgagatctttcctcctggttactgtgaagcctgttggtttgctgctgtcgtttttgaggagggcccatgggggtaggagcagttgaacctgggaacaaacctcacttgagctgtgcctagacaatgtgaattcctgtgttgctaacagaagtggcctgtaagctcctgtgctccggagggaagcatttcctggtaggctttg

SEQ ID NO: 117 - DNA, 535 bp, Homo Sapiens
gctgaaggcagatgtcgtcccaaagacagctgagaacttcagagccctgtgcactggtgagaagggcttcggctacaaaggctccaccttccacagggtgatcccttccttcatgtgccaggcgggcgacttcaccaaccacaatggcacaggcgggaagtccatctacggaagccgctttcctgacgagaactttacactgaagcacgtggggccaggtgtcctgtccatggctaatgctggtcctaacaccaacggctcccagttcttcatctgcaccataaagacagactggttggatggcaagcatgttgtgttcggtcacgtcaaagagggcatggacgtcgtgaagaaaatagaatctttcggctctaagagtgggaggacatccaagaagattgtcatcacagactgtggccagttgagctaatctgtggccagggtgctggcatggtggcagctgcaaatgtccatgcacccaggtggccgcgttgggctgtcagccaaggtgcctgaaacgatacgtgtgcccact

SEQ ID NO: 118 - DNA, 484 bp, Homo Sapiens
ggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaat

SEQ ID NO: 119 - DNA, 495 bp, Homo Sapiens
gaacaagcgtcctggggcatttgctatttacctggagccttggcatttagacatctttgaattccttgatttaaagaagaacacaggaaaggaagagcagcgtgccagagatcttttctttgctctttggattccggatctcttcatgaaacgagtggagactaatcaggactggtctttgatgtgtccaaatgagtgtcctggtctggatgaggtttggggagaggaatttgagaaactatatgcaagttatgagaaacaaggtcgtgtccgcaaagttgtaaaagctcagcagctttggtatgccatcattgagtctcagacggaaacaggcaccccgtatatgctctacaaagattcctgtaatcgaaagagcaaccagcagaacctgggaaccatcaaatgcagcaacctgtgcacagaaatagtggagtacaccagcaaagatgaggttgctgtttgtaatttggcttccctggccctgaatatgtatgt

SEQ ID NO: 120 - DNA, 438 bp, Homo Sapiens
gcccctggagtcgcggagaaagggccgtaaccggaggacccacgcccctgagcctcgcgctgagcgggggccgcgcagcgcaacgcactggtgaccagactgtccccacgccgggaaccaagcaggagacgacaggcgagagaggagccagacagaccctgaaaagaaggacgggttggggccgggcacattgggggtcaccggccgatggagacaccaaccgacaggccctggctgagggcagctgcgcgggcttatttattaacaggataacccttgaatgtagcagccccgggagggcggcacaggtcgggcgcaggattcagccggagggaagggacggggaagccgagctccagagcaacgaccagggccgaggaggtgcctggagtgcccaccctgggagacagaccccacctccttgggtagtgagcagtg

SEQ ID NO: 121 - DNA, 447 bp, Homo Sapiens
  misc_feature (116)..(116): n is a, c, g, or t
ggaactacggggcttacaggagcttttgtgtgcctggtagaaactatttctgttccagtcacattgccatcactcttgtactgcctgccaccgcggaggaggctggtgacaggccnaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaangtcagatgcgtgcccagctcttcaccccccaatctcttggtggggaggggtgtacctaaatatttatcatatccttgcccttgag

SEQ ID NO: 122 - DNA, 323 bp, Homo Sapiens
aaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggta

SEQ ID NO: 123 - DNA, 499 bp, Homo Sapiens
gtatcaggcttcaattccattatgttttaatgttgtctctgaagatgacttgtgatttttttttcttttttttaaaccatgaagagccgtttgacagagcatgctctgcgttgttggtttcaccagcttctgccctcacatgcacagggatttaacaacaaaaatataactacaacttcccttgtagtctcttatataagtagagtccttggtactctgccctcctgtcagtagtggcaggatctattggcatattcgggagcttcttagagggatgaggttctttgaacacagtgaaaatttaaattagtaacttttttgcaagcagtttattgactgttattgctaagaagaagtaagaaagaaaaagcctgttggcaatcttggttatttctttaagatttctggcagtgtgggatggatgaatgaagtggaatgtgaactttgggcaagttaaatgggacagccttccatgttcatttgtctacctcttaactga

SEQ ID NO: 124 - DNA, 328 bp, Homo Sapiens
taattttagattcgccttacaatgtaaatcttcacattggagataatattggttggaccttgcccatcttcactctagccttcgtatttgtgaaggactcagccaccttccttcttcaccccatgcttctcaccaaatttttgttgtcattgagggcacttggataactcaagttgatatttatagctgatcaatctatatgtgtcacagaactatgctgcctaaagtgatcttggctccttaatggtccttttggccccttggatagttaacagctgagtaattctaatctcttctgtgttttccttgccttaaccacaaattgtgg

SEQ ID NO: 125 - DNA, 489 bp, Homo Sapiens
gagatacagaacttggtgacccatgtattgcataagctaaagcaacacagacactcctaggcaaagtttttgtttgtgaatagtacttgcaaaacttgtaaattagcagatgacttttttccattgttttctccagagagaatgtgctatatttttgtatatacaataatatttgcaactgtgaaaaacaagttgtgccatactacatggcacagacacaaaatattatactaatatgttgtacattcggaagaatgtgaatcaatcagtatgtttttagattgtattttgccttacagaaagcctttattgtaagactctgatttccctttggacttcatgtatattgtacagttacagtaaaattcaacctttattttctaattttttcaacatattgtttagtgtaaagaatatttatttgaagttttattattttataaaaaagaatatttattttaagaggcatcttacaaattttgccccttt

SEQ ID NO: 126 - DNA, 503 bp, Homo Sapiens
gcggcatgtgaccatcattgaactggtgggacagccacctcaggaggtggggcgcatccgggagcaacagctgtcagccaacatcatcgaggagctcaggcaatttcagcgcctcactcgctcctacttcaacatggtgttgattgacaagcagggtattgaccgagaccgctacatggaacctgtcacccccgaggaaatcttcacattcattgatgactacctactgagcaatcaggagttgacccagcgtcgggagcaaagggacatatgcgagtgaacttgagccagggcatggttaaagtcaagggaaaagctcctctagttagctgaaactgggacctaataaaaggaggaaatgttttcccacagttctagggacaggactctgaggtgggtgagtttgacaaatcctgcagtgtttccaggcatccttttaggactgtgtaatagtttccctagaagctaggtagggactgaggacaggccttgggcagtgggtt

SEQ ID NO: 127 - DNA, 436 bp, Homo Sapiens
agactgggcgaaaggctgtccggagggcagaccaggtgccttgccgcagagaaaacaccaaagtctcctgttcgctcataaagaagtttttgggatgggagagaatccagaccatcttggggcagccaggcccttgccttcatttttacagaggtagcacaactgattccaacacaaaaccccttcccctttttaaaatgatttctgttctaatgccatagatcaaaggcctcagaaaccattgtgtgtttcctctttgaagcaatgacaagcactttactttcacggtggtttttgttttttcttattgctgtggaacctcttttggaggacgttaaaggcgtgttttacttgtttttttaagagtgtgtgatgtgtgttttgtagatttcttgacagtgctgtaatacagacggcaatgcaatagcctatttaa

SEQ ID NO: 128 - DNA, 497 bp, Homo Sapiens
cctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagt

SEQ ID NO: 129 - DNA, 321 bp, Homo Sapiens
gtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggctt

SEQ ID NO: 130 - DNA, 553 bp, Homo Sapiens
tttgcctgcagtttcttgtgtagatttgaaaattgtataccaatgtgttttctgtagactctaagatacactgcactttgtttagaaaaaaaactgaagatgaaatatatattgtaaagaagggatattaagaatcttagataacttcttgaaaaagatggcttatgtcatcagtaaagtacctttatgttatgaggatataatgtgtgctttattgaattagaaaattagtgaccattattcacaggtggacaaatgttgtcctgttaatttataggagttttttggggatgtggaggtagttgggtagaaaaattattagaacattcacttttgttaacagtatttctcttttattctgttatatagtggatgatatacacagtggcaaaacaaaagtacattgcttaaaatatatagtgaaaaatgtcactatatcttcccatttaacattgtttttgtatattgggtgtagatttctgacatcaaaacttggacccttggaaaacaaaagttttaattaaaaaaaatccttgtgacttacaatttgcac

SEQ ID NO: 131 - DNA, 419 bp, Homo Sapiens
gagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatgggg

SEQ ID NO: 132 - DNA, 414 bp, Homo Sapiens
ttgttgtcgttgcttgtttgaagaaaatcatgacattccaagttgacattttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttg

SEQ ID NO: 133 - DNA, 419 bp, Homo Sapiens
aggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggcc

SEQ ID NO: 134 - DNA, 493 bp, Homo Sapiens
gactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatg

SEQ ID NO: 135 - DNA, 567 bp, Homo Sapiens
gagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattat

SEQ ID NO: 136 - DNA, 479 bp, Homo Sapiens
  misc_feature (441)..(441): n is a, c, g, or t
accaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttganaagattcttaatagctggctgtaaatactgcttggttt

SEQ ID NO: 137 - DNA, 490 bp, Homo Sapiens
gtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacct

SEQ ID NO: 138 - DNA, 248 bp, Homo Sapiens
ctctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggc

SEQ ID NO: 139 - DNA, 405 bp, Homo Sapiens
  misc_feature (64)..(64): n is a, c, g, or t
gctcacccagcagatgttcgatgccaagaacatgatggccgcctgcgacccgcgccacggccgnctanctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtacc

SEQ ID NO: 140 - DNA, 407 bp, Homo Sapiens
gatgcctaaccaaggactagagctccttcttgagatctaaatctaaagtaaatgtgcattaaagcagtgtgcttcaaaggcatcagacgatgaaagcaacataccacaactaggagttatttctcaaacttaaatgtcctctgggaatccagacttaaaaataagagcaaacttaacacactatccattttcgagcaaacttaacccactatatccattttgctcatgtgttttatgcaaccagctttccatcaaatcctcaatccttgaatccaggtaaaaggttaattatcctaggattagtgaatgattcaatgaagctttcttgaaaacaaacataggagtgtaatgtactattatgtttgtatcctgttttagtttataaagcactttcacatacattatgg

SEQ ID NO: 141 - DNA, 518 bp, Homo Sapiens
acccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgagctacattcatgggagcagccaggctcagtttgtggcagaatcacacattattctggtactgaatgccgctatcaccatggggatggttcttctaaatgaagcagcaacttcgaaaggcgatgttggaaaaagacggataatttgcctagtgggattgggcctggtggtcttcttcttcagttttctactttcaatatttcgttccaagtaccacggctatccttatagtgatctggactttgagtgagaagatgtgatttggaccatggcacttaaaaactctataacctcagccttttaat

SEQ ID NO: 142 - DNA, 443 bp, Homo Sapiens
ctttgctatgacttctggccagatgtggaaccatatccgtggacctccatatgctcataagaacccacacaatggacaagtgagctacattcatgggagcagccaggctcagtttgtggcagaatcacacattattctggtactgaatgccgctatcaccatggggatggttcttctaaatgaagcagcaacttcgaaaggcgatgttggaaaaagacggataatttgcctagtgggattgggcctggtggtcttcttcttcagttttctactttcaatatttcgttccaagtaccacggctatccttatagctttttaattaaatgaagccaagtgggatttgcataaagtgaatgtttaccatgaagataaactgttcctgactttatactattttgaattcattcatttcattgtgatcagctagcttattcttgtgtac